SEQUENCE LISTING (12 sequences)

SEQ ID NO: 1 - DNA, 3957 bp, Severe acute respiratory syndrome coronavirus 2
attttggcaaagaattcgctagtggatccaggccgcctgggcccgttaacaccatgttcgtattccttgtcctcctccccctcgtcagctcccagtgtgtcaatctgacaactcgaactcagctcccacccgcatacaccaatagttttacccgaggagtctattatcccgacaaagttttcaggtctagtgtgttgcatagcacgcaggacctgttcttgccgtttttttcaaatgtgacatggttccatgccatccatgtctctggtacgaacgggaccaaaaggtttgataaccccgttcttccatttaacgacggggtttatttcgcctctactgaaaagtctaatataatacgaggttggatttttgggacaacactggactctaaaacgcaatctctcctcatagtaaataatgcgacgaatgtcgtaatcaaagtatgtgagtttcagttttgcaacgacccttttcttggtgtatactatcacaagaacaacaagtcttggatggaaagtgaatttagagtctacagctccgcaaacaactgtacctttgagtacgtcagtcagcccttccttatggacctcgaaggcaaacaggggaacttcaagaatctgcgcgagttcgtgttcaagaatatcgacggctatttcaagatatattcaaaacatacaccaatcaatttggtacgcgatttgccgcagggcttctctgctcttgagcctctcgtagatttgcctatagggatcaatattactcgctttcaaactctccttgcccttcatcggtcctacctgacgccaggcgacagctccagtgggtggacagctggtgcagcggcttactacgtcggctacctccaacccaggacttttttgctgaaatataatgagaatggtacgattactgacgccgtcgattgtgccctcgacccattgagcgaaactaaatgcaccctgaaaagctttacggttgagaaagggatatatcagacatctaattttcgcgttcaacccacggaatccattgtacgatttcccaacattaccaatttgtgtccttttggtgaggtatttaatgcaaccagattcgcgagtgtgtatgcctggaataggaaaagaatttctaactgtgtagcagactacagcgttctgtataatagtgcgtcctttagtacgttcaagtgctacggagtaagcccgacaaagttgaacgatttgtgtttcactaacgtctatgccgatagcttcgtcatcaggggagacgaagtaaggcaaattgcaccaggccagaccggaaaaatagccgactacaactataagttgccggacgatttcaccgggtgtgtcatcgcgtggaatagtaataacctggactccaaggtgggcggcaactacaactacctgtaccgattgttccgcaagtctaacctcaagccctttgaacgagacatatccaccgagatataccaagcgggaagcactccctgtaatggcgttgaaggtttcaactgctattttcctctccagtcctatggcttccaacctaccaatggagtaggctatcaaccgtatcgagtcgtggtgctgagtttcgagctcctgcacgctccagcgaccgtttgtggtcctaagaaaagtacaaacctcgtaaaaaataagtgcgtaaactttaactttaacggtctcacaggaacgggagttcttacagagtccaataagaagttcctgccctttcagcagtttgggcgcgacatcgcggacaccacagatgcagtcagggacccgcagactcttgagatcctggacatcaccccatgctcttttggcggtgtgagtgtcattacacccgggaccaacaccagcaatcaggttgctgtgctctatcaagacgtcaactgtacggaagtcccagttgcaatacacgcggaccagttgacgccgacgtggcgggtttattctaccggctcaaacgtctttcaaactagagccggttgtttgatcggggcggagcatgtgaacaattcatacgaatgtgatattcccataggagccggaatatgtgcttcttaccagacccaaaccaactctccgagacgagcccggtccgtagccagtcaaagcataattgcgtacaccatgagcctcggtgcagaaaactcagttgcgtactcaaataactccattgccatcccaacaaattttaccatatcagtgaccactgaaatccttccagtgagcatgactaaaacaagtgtagattgcacaatgtatatttgcggagactcaactgagtgctctaacctcctgttgcagtacggttcattctgtacccagctcaatcgcgcacttacgggaatagcagtagaacaagataagaatactcaggaagtcttcgctcaagtaaagcaaatctacaagacgccccccattaaggactttgggggttttaattttagccaaatactcccggaccccagtaaaccctctaagaggtcatttatagaagacctgttgtttaataaagttactctggccgatgctggctttattaaacaatacggtgattgtttgggcgacatcgcggcgcgggatctgatatgcgcccaaaaatttaacggtctcactgtgttgccgcctttgctcacagacgagatgatagctcagtacacatcagcgttgttggcgggtaccataacgtctggatggacattcggagcaggggcggccttgcaaataccattcgcaatgcaaatggcatacaggttcaacgggataggtgttacacagaatgtactttacgagaaccagaaattgattgcaaaccagtttaactctgcgattggcaaaattcaggatagcctcagctccaccgcgtctgcactgggaaaactccaagatgttgtgaaccaaaatgcccaagccctcaatacacttgtgaagcaactctccagcaatttcggtgcaataagtagtgtgctgaacgacatactttctcggttggacaaagtagaggcggaggtgcaaatcgacagactcataacaggtcgactgcaatcattgcaaacgtatgttactcagcaattgatacgggcggcggagattcgagcgtctgccaacctcgccgcgactaaaatgtctgagtgtgtgctcggtcagtcaaaacgcgtggacttctgtggaaagggataccatctcatgtccttccctcagtctgcccctcacggagtagtctttcttcatgtgacctatgttccagcacaggagaaaaattttaccacagcgcccgcgatatgccacgatggcaaagctcattttccgcgagagggagtatttgtctctaacggcacccattggtttgtaacccagcgcaatttttacgaacctcagataatcaccactgacaacacctttgttagtggtaattgtgatgtagtgatcgggatagtgaacaatacagtgtacgaccccctgcaacctgagctggactcctttaaagaagaactcgacaagtacttcaaaaaccacacttccccggatgtcgatttgggtgatatatctggcatcaatgcgagtgtagtcaacatacagaaggaaatcgacaggctcaatgaagtagcaaaaaacctgaatgaatccctcatagacctccaagagctgggcaaatatgagcagtacattaaatggccctggtacatttggctgggcttcatcgctggcctgatagcgatagtgatggttactattatgctctgctgtatgacgagttgttgcagctgcctcaaagggtgttgttcttgcggctcctgttgtaagttcgacgaggatgattcagagcccgttcttaagggtgtaaaactccattacactggaggaggaggttacccatacgatgttccagattacgcttagtaaactagtaacgtctcgagtttaaacctggaaaaacatggag

SEQ ID NO: 2 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - DNA, 29664 bp, Artificial Sequence
  Description of Artificial Sequence Synthetic polynucleotide
ttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccttgcttggagaaaagctgtctttatttcaccttataattcacagaatgctgtagcctcaaagattttgggactaccaactcaaactgttgattcatcacagggctcagaatgtgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactttttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacata

SEQ ID NO: 4 - protein, 1273 aa, Severe acute respiratory syndrome coronavirus 2; identical to SEQ ID NO: 2

SEQ ID NO: 5 - DNA, 27 bp, Artificial Sequence
  Description of Artificial Sequence Synthetic oligonucleotide
tacccatacgatgttccagattacgct

SEQ ID NO: 6 - protein, 9 aa, Artificial Sequence
  Description of Artificial Sequence Synthetic peptide
YPYDVPDYA

SEQ ID NO: 7 - DNA, 3819 bp, Artificial Sequence
  Description of Artificial Sequence Synthetic polynucleotide
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacaca

SEQ ID NO: 8 - DNA, 9598 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcaacttttaaaagaaaaggggggattggggggtacagtgcaggggaaagaatagtagacataatagcaacagacatacaaactaaagaattacaaaaacaaattacaaaattcaaaattttatcgatgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctgtcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagtggatccaccggtggccgcctgggcccgttaacgtctcgagtttaaacctggaaaaacatggagcaatcacaagtagcaatacagcagctaccaatgctgattgtgcctggctagaagcacaagaggaggaggaggtgggttttccagtcacacctcagacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggccgctttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 9 - DNA, 3858 bp, Artificial Sequence
  Description of Artificial Sequence Synthetic polynucleotide
atgttcgtattccttgtcctcctccccctcgtcagctcccagtgtgtcaatctgacaactcgaactcagctcccacccgcatacaccaatagttttacccgaggagtctattatcccgacaaagttttcaggtctagtgtgttgcatagcacgcaggacctgttcttgccgtttttttcaaatgtgacatggttccatgccatccatgtctctggtacgaacgggaccaaaaggtttgataaccccgttcttccatttaacgacggggtttatttcgcctctactgaaaagtctaatataatacgaggttggatttttgggacaacactggactctaaaacgcaatctctcctcatagtaaataatgcgacgaatgtcgtaatcaaagtatgtgagtttcagttttgcaacgacccttttcttggtgtatactatcacaagaacaacaagtcttggatggaaagtgaatttagagtctacagctccgcaaacaactgtacctttgagtacgtcagtcagcccttccttatggacctcgaaggcaaacaggggaacttcaagaatctgcgcgagttcgtgttcaagaatatcgacggctatttcaagatatattcaaaacatacaccaatcaatttggtacgcgatttgccgcagggcttctctgctcttgagcctctcgtagatttgcctatagggatcaatattactcgctttcaaactctccttgcccttcatcggtcctacctgacaccaggcgacagctccagtgggtggacagctggtgcagcggcttactacgtcggctacctccaacccaggacttttttgctgaaatataatgagaatggtacgattactgacgccgtcgattgtgccctcgacccattgagcgaaactaaatgcaccctgaaaagctttacggttgagaaagggatatatcagacatctaattttcgcgttcaacccacggaatccattgtacgatttcccaacattaccaatttgtgtccttttggtgaggtatttaatgcaaccagattcgcgagtgtgtatgcctggaataggaaaagaatttctaactgtgtagcagactacagcgttctgtataatagtgcgtcctttagtacgttcaagtgctacggagtaagcccgacaaagttgaacgatttgtgtttcactaacgtctatgccgatagcttcgtcatcaggggagacgaagtaaggcaaattgcaccaggccagaccggaaaaatagccgactacaactataagttgccggacgatttcaccgggtgtgtcatcgcgtggaatagtaataacctggactccaaggtgggcggcaactacaactacctgtaccgattgttccgcaagtctaacctcaagccctttgaacgagacatatccaccgagatataccaagcgggaagcactccctgtaatggcgttgaaggtttcaactgctattttcctctccagtcctatggcttccaacctaccaatggagtaggctatcaaccgtatcgagtcgtggtgctgagtttcgagctcctgcacgctccagcgaccgtttgtggtcctaagaaaagtacaaacctcgtaaaaaataagtgcgtaaactttaactttaacggtctcacaggaacgggagttcttacagagtccaataagaagttcctgccctttcagcagtttgggcgcgacatcgcggacaccacagatgcagtcagggacccgcagactcttgagatcctggacatcaccccatgctcttttggcggtgtgagtgtcattacacccgggaccaacaccagcaatcaggttgctgtgctctatcaagacgtcaactgtacggaagtcccagttgcaatacacgcggaccagttgacgccgacgtggcgggtttattctaccggctcaaacgtctttcaaactagagccggttgtttgatcggggcggagcatgtgaacaattcatacgaatgtgatattcccataggagccggaatatgtgcttcttaccagacccaaaccaactctccgagacgagcccggtccgtagccagtcaaagcataattgcgtacaccatgagcctcggtgcagaaaactcagttgcgtactcaaataactccattgccatcccaacaaattttaccatatcagtgaccactgaaatccttccagtgagcatgactaaaacaagtgtagattgcacaatgtatatttgcggagactcaactgagtgctctaacctcctgttgcagtacggttcattctgtacccagctcaatcgcgcacttacgggaatagcagtagaacaagataagaatactcaggaagtcttcgctcaagtaaagcaaatctacaagacgccccccattaaggactttgggggttttaattttagccaaatactcccggaccccagtaaaccctctaagaggtcatttatagaagacctgttgtttaataaagttactctggccgatgctggctttattaaacaatacggtgattgtttgggcgacatcgcggcgcgggatctgatatgcgcccaaaaatttaacggtctcactgtgttgccgcctttgctcacagacgagatgatagctcagtacacatcagcgttgttggcgggtaccataacgtctggatggacattcggagcaggggcggccttgcaaataccattcgcaatgcaaatggcatacaggttcaacgggataggtgttacacagaatgtactttacgagaaccagaaattgattgcaaaccagtttaactctgcgattggcaaaattcaggatagcctcagctccaccgcgtctgcactgggaaaactccaagatgttgtgaaccaaaatgcccaagccctcaatacacttgtgaagcaactctccagcaatttcggtgcaataagtagtgtgctgaacgacatactttctcggttggacaaagtagaggcggaggtgcaaatcgacagactcataacaggtcgactgcaatcattgcaaacgtatgttactcagcaattgatacgggcggcggagattcgagcgtctgccaacctcgccgcgactaaaatgtctgagtgtgtgctcggtcagtcaaaacgcgtggacttctgtggaaagggataccatctcatgtccttccctcagtctgcccctcacggagtagtctttcttcatgtgacctatgttccagcacaggagaaaaattttaccacagcgcccgcgatatgccacgatggcaaagctcattttccgcgagagggagtatttgtctctaacggcacccattggtttgtaacccagcgcaatttttacgaacctcagataatcaccactgacaacacctttgttagtggtaattgtgatgtagtgatcgggatagtgaacaatacagtgtacgaccccctgcaacctgagctggactcctttaaagaagaactcgacaagtacttcaaaaaccacacttccccggatgtcgatttgggtgatatatctggcatcaatgcgagtgtagtcaacatacagaaggaaatcgacaggctcaatgaagtagcaaaaaacctgaatgaatccctcatagacctccaagagctgggcaaatatgagcagtacattaaatggccctggtacatttggctgggcttcatcgctggcctgatagcgatagtgatggttactattatgctctgctgtatgacgagttgttgcagctgcctcaaagggtgttgttcttgcggctcctgttgtaagttcgacgaggatgattcagagcccgttcttaagggtgtaaaactccattacactggaggaggaggttacccatacgatgttccagattacgct

SEQ ID NO: 10 - DNA, 13467 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacttggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaagcaagaatcctagctgtggaaagatacctaaaggatcaacagctcctagggatttggggttgctctggaaaactcatttgcaccactgctgtgccttggaatgctagttggagtaataaatctctggaacagatctggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggacccgacaggcccgaaggaatagaagaagaaggtggagagagagacagagacagatccattcgattagtgaacggatcaacttttaaaagaaaaggggggattggggggtacagtgcaggggaaagaatagtagacataatagcaacagacatacaaactaaagaattacaaaaacaaattacaaaaattcaaaattttatcgatgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattcgctagtggatccaggccgcctgggcccgttaacaccatgttcgtattccttgtcctcctccccctcgtcagctcccagtgtgtcaatctgacaactcgaactcagctcccacccgcatacaccaatagttttacccgaggagtctattatcccgacaaagttttcaggtctagtgtgttgcatagcacgcaggacctgttcttgccgtttttttcaaatgtgacatggttccatgccatccatgtctctggtacgaacgggaccaaaaggtttgataaccccgttcttccatttaacgacggggtttatttcgcctctactgaaaagtctaatataatacgaggttggatttttgggacaacactggactctaaaacgcaatctctcctcatagtaaataatgcgacgaatgtcgtaatcaaagtatgtgagtttcagttttgcaacgacccttttcttggtgtatactatcacaagaacaacaagtcttggatggaaagtgaatttagagtctacagctccgcaaacaactgtacctttgagtacgtcagtcagcccttccttatggacctcgaaggcaaacaggggaacttcaagaatctgcgcgagttcgtgttcaagaatatcgacggctatttcaagatatattcaaaacatacaccaatcaatttggtacgcgatttgccgcagggcttctctgctcttgagcctctcgtagatttgcctatagggatcaatattactcgctttcaaactctccttgcccttcatcggtcctacctgacaccaggcgacagctccagtgggtggacagctggtgcagcggcttactacgtcggctacctccaacccaggacttttttgctgaaatataatgagaatggtacgattactgacgccgtcgattgtgccctcgacccattgagcgaaactaaatgcaccctgaaaagctttacggttgagaaagggatatatcagacatctaattttcgcgttcaacccacggaatccattgtacgatttcccaacattaccaatttgtgtccttttggtgaggtatttaatgcaaccagattcgcgagtgtgtatgcctggaataggaaaagaatttctaactgtgtagcagactacagcgttctgtataatagtgcgtcctttagtacgttcaagtgctacggagtaagcccgacaaagttgaacgatttgtgtttcactaacgtctatgccgatagcttcgtcatcaggggagacgaagtaaggcaaattgcaccaggccagaccggaaaaatagccgactacaactataagttgccggacgatttcaccgggtgtgtcatcgcgtggaatagtaataacctggactccaaggtgggcggcaactacaactacctgtaccgattgttccgcaagtctaacctcaagccctttgaacgagacatatccaccgagatataccaagcgggaagcactccctgtaatggcgttgaaggtttcaactgctattttcctctccagtcctatggcttccaacctaccaatggagtaggctatcaaccgtatcgagtcgtggtgctgagtttcgagctcctgcacgctccagcgaccgtttgtggtcctaagaaaagtacaaacctcgtaaaaaataagtgcgtaaactttaactttaacggtctcacaggaacgggagttcttacagagtccaataagaagttcctgccctttcagcagtttgggcgcgacatcgcggacaccacagatgcagtcagggacccgcagactcttgagatcctggacatcaccccatgctcttttggcggtgtgagtgtcattacacccgggaccaacaccagcaatcaggttgctgtgctctatcaagacgtcaactgtacggaagtcccagttgcaatacacgcggaccagttgacgccgacgtggcgggtttattctaccggctcaaacgtctttcaaactagagccggttgtttgatcggggcggagcatgtgaacaattcatacgaatgtgatattcccataggagccggaatatgtgcttcttaccagacccaaaccaactctccgagacgagcccggtccgtagccagtcaaagcataattgcgtacaccatgagcctcggtgcagaaaactcagttgcgtactcaaataactccattgccatcccaacaaattttaccatatcagtgaccactgaaatccttccagtgagcatgactaaaacaagtgtagattgcacaatgtatatttgcggagactcaactgagtgctctaacctcctgttgcagtacggttcattctgtacccagctcaatcgcgcacttacgggaatagcagtagaacaagataagaatactcaggaagtcttcgctcaagtaaagcaaatctacaagacgccccccattaaggactttgggggttttaattttagccaaatactcccggaccccagtaaaccctctaagaggtcatttatagaagacctgttgtttaataaagttactctggccgatgctggctttattaaacaatacggtgattgtttgggcgacatcgcggcgcgggatctgatatgcgcccaaaaatttaacggtctcactgtgttgccgcctttgctcacagacgagatgatagctcagtacacatcagcgttgttggcgggtaccataacgtctggatggacattcggagcaggggcggccttgcaaataccattcgcaatgcaaatggcatacaggttcaacgggataggtgttacacagaatgtactttacgagaaccagaaattgattgcaaaccagtttaactctgcgattggcaaaattcaggatagcctcagctccaccgcgtctgcactgggaaaactccaagatgttgtgaaccaaaatgcccaagccctcaatacacttgtgaagcaactctccagcaatttcggtgcaataagtagtgtgctgaacgacatactttctcggttggacaaagtagaggcggaggtgcaaatcgacagactcataacaggtcgactgcaatcattgcaaacgtatgttactcagcaattgatacgggcggcggagattcgagcgtctgccaacctcgccgcgactaaaatgtctgagtgtgtgctcggtcagtcaaaacgcgtggacttctgtggaaagggataccatctcatgtccttccctcagtctgcccctcacggagtagtctttcttcatgtgacctatgttccagcacaggagaaaaattttaccacagcgcccgcgatatgccacgatggcaaagctcattttccgcgagagggagtatttgtctctaacggcacccattggtttgtaacccagcgcaatttttacgaacctcagataatcaccactgacaacacctttgttagtggtaattgtgatgtagtgatcgggatagtgaacaatacagtgtacgaccccctgcaacctgagctggactcctttaaagaagaactcgacaagtacttcaaaaaccacacttccccggatgtcgatttgggtgatatatctggcatcaatgcgagtgtagtcaacatacagaaggaaatcgacaggctcaatgaagtagcaaaaaacctgaatgaatccctcatagacctccaagagctgggcaaatatgagcagtacattaaatggccctggtacatttggctgggcttcatcgctggcctgatagcgatagtgatggttactattatgctctgctgtatgacgagttgttgcagctgcctcaaagggtgttgttcttgcggctcctgttgtaagttcgacgaggatgattcagagcccgttcttaagggtgtaaaactccattacactggaggaggaggttacccatacgatgttccagattacgcttagtaaactagtaacgtctcgagtttaaacctggaaaaacatggagcaatcacaagtagcaacacagcagctaccaatgctgcttgtgcctggctagaagcacaagaggaggaggaggtgggttttccagtcacacctcagacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattgacagaactacacaccagggccaggggtcagatatccactgacctttggatggtgctacaagctagtaccagttgagccagataagatagaagaggccaataaaggagagaacaccagcttgttacaccctgtgagcctgcatgggatggatgacccggagagagaagtgttagagtggaggtttgacagccgcctagcatttcatcacgtggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggccgctttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 11 - DNA, 3819 bp, Artificial Sequence; identical to SEQ ID NO: 7
  Description of Artificial Sequence Synthetic polynucleotide

SEQ ID NO: 12 - DNA, 3819 bp, Severe acute respiratory syndrome coronavirus 2
atgttcgtattccttgtcctcctccccctcgtcagctcccagtgtgtcaatctgacaactcgaactcagctcccacccgcatacaccaatagttttacccgaggagtctattatcccgacaaagttttcaggtctagtgtgttgcatagcacgcaggacctgttcttgccgtttttttcaaatgtgacatggttccatgccatccatgtctctggtacgaacgggaccaaaaggtttgataaccccgttcttccatttaacgacggggtttatttcgcctctactgaaaagtctaatataatacgaggttggatttttgggacaacactggactctaaaacgcaatctctcctcatagtaaataatgcgacgaatgtcgtaatcaaagtatgtgagtttcagttttgcaacgacccttttcttggtgtatactatcacaagaacaacaagtcttggatggaaagtgaatttagagtctacagctccgcaaacaactgtacctttgagtacgtcagtcagcccttccttatggacctcgaaggcaaacaggggaacttcaagaatctgcgcgagttcgtgttcaagaatatcgacggctatttcaagatatattcaaaacatacaccaatcaatttggtacgcgatttgccgcagggcttctctgctcttgagcctctcgtagatttgcctatagggatcaatattactcgctttcaaactctccttgcccttcatcggtcctacctgacgccaggcgacagctccagtgggtggacagctggtgcagcggcttactacgtcggctacctccaacccaggacttttttgctgaaatataatgagaatggtacgattactgacgccgtcgattgtgccctcgacccattgagcgaaactaaatgcaccctgaaaagctttacggttgagaaagggatatatcagacatctaattttcgcgttcaacccacggaatccattgtacgatttcccaacattaccaatttgtgtccttttggtgaggtatttaatgcaaccagattcgcgagtgtgtatgcctggaataggaaaagaatttctaactgtgtagcagactacagcgttctgtataatagtgcgtcctttagtacgttcaagtgctacggagtaagcccgacaaagttgaacgatttgtgtttcactaacgtctatgccgatagcttcgtcatcaggggagacgaagtaaggcaaattgcaccaggccagaccggaaaaatagccgactacaactataagttgccggacgatttcaccgggtgtgtcatcgcgtggaatagtaataacctggactccaaggtgggcggcaactacaactacctgtaccgattgttccgcaagtctaacctcaagccctttgaacgagacatatccaccgagatataccaagcgggaagcactccctgtaatggcgttgaaggtttcaactgctattttcctctccagtcctatggcttccaacctaccaatggagtaggctatcaaccgtatcgagtcgtggtgctgagtttcgagctcctgcacgctccagcgaccgtttgtggtcctaagaaaagtacaaacctcgtaaaaaataagtgcgtaaactttaactttaacggtctcacaggaacgggagttcttacagagtccaataagaagttcctgccctttcagcagtttgggcgcgacatcgcggacaccacagatgcagtcagggacccgcagactcttgagatcctggacatcaccccatgctcttttggcggtgtgagtgtcattacacccgggaccaacaccagcaatcaggttgctgtgctctatcaagacgtcaactgtacggaagtcccagttgcaatacacgcggaccagttgacgccgacgtggcgggtttattctaccggctcaaacgtctttcaaactagagccggttgtttgatcggggcggagcatgtgaacaattcatacgaatgtgatattcccataggagccggaatatgtgcttcttaccagacccaaaccaactctccgagacgagcccggtccgtagccagtcaaagcataattgcgtacaccatgagcctcggtgcagaaaactcagttgcgtactcaaataactccattgccatcccaacaaattttaccatatcagtgaccactgaaatccttccagtgagcatgactaaaacaagtgtagattgcacaatgtatatttgcggagactcaactgagtgctctaacctcctgttgcagtacggttcattctgtacccagctcaatcgcgcacttacgggaatagcagtagaacaagataagaatactcaggaagtcttcgctcaagtaaagcaaatctacaagacgccccccattaaggactttgggggttttaattttagccaaatactcccggaccccagtaaaccctctaagaggtcatttatagaagacctgttgtttaataaagttactctggccgatgctggctttattaaacaatacggtgattgtttgggcgacatcgcggcgcgggatctgatatgcgcccaaaaatttaacggtctcactgtgttgccgcctttgctcacagacgagatgatagctcagtacacatcagcgttgttggcgggtaccataacgtctggatggacattcggagcaggggcggccttgcaaataccattcgcaatgcaaatggcatacaggttcaacgggataggtgttacacagaatgtactttacgagaaccagaaattgattgcaaaccagtttaactctgcgattggcaaaattcaggatagcctcagctccaccgcgtctgcactgggaaaactccaagatgttgtgaaccaaaatgcccaagccctcaatacacttgtgaagcaactctccagcaatttcggtgcaataagtagtgtgctgaacgacatactttctcggttggacaaagtagaggcggaggtgcaaatcgacagactcataacaggtcgactgcaatcattgcaaacgtatgttactcagcaattgatacgggcggcggagattcgagcgtctgccaacctcgccgcgactaaaatgtctgagtgtgtgctcggtcagtcaaaacgcgtggacttctgtggaaagggataccatctcatgtccttccctcagtctgcccctcacggagtagtctttcttcatgtgacctatgttccagcacaggagaaaaattttaccacagcgcccgcgatatgccacgatggcaaagctcattttccgcgagagggagtatttgtctctaacggcacccattggtttgtaacccagcgcaatttttacgaacctcagataatcaccactgacaacacctttgttagtggtaattgtgatgtagtgatcgggatagtgaacaatacagtgtacgaccccctgcaacctgagctggactcctttaaagaagaactcgacaagtacttcaaaaaccacacttccccggatgtcgatttgggtgatatatctggcatcaatgcgagtgtagtcaacatacagaaggaaatcgacaggctcaatgaagtagcaaaaaacctgaatgaatccctcatagacctccaagagctgggcaaatatgagcagtacattaaatggccctggtacatttggctgggcttcatcgctggcctgatagcgatagtgatggttactattatgctctgctgtatgacgagttgttgcagctgcctcaaagggtgttgttcttgcggctcctgttgtaagttcgacgaggatgattcagagcccgttcttaagggtgtaaaactccattacact

(19) United States
(12) Patent Application Publication
(10) Pub. No.: US 2022/0048954 A1
(43) Pub. Date: Feb. 17, 2022

(54) SARS-COV2 VACCINE VECTOR METHODS AND COMPOSITIONS

(71) Applicant: Salk Institute for Biological Studies, La Jolla, CA (US)

(72) Inventors: Gerald Pao, San Diego, CA (US); Junko Ogawa, San Diego, CA (US)

(21) Appl. No.: 17/240,902

(22) Filed: Apr. 26, 2021

Related U.S. Application Data
(60) Provisional application No. 63/015,352, filed on Apr. 24, 2020.

(51) Int. Cl. C07K 14/005; C12N 7/00; C12N 15/86

(52) U.S. Cl. CPC C07K 14/005; C12N 7/00; C12N 2740/15043; C12N 2770/20022; C12N 15/86

(57) ABSTRACT

Provided herein are recombinant nucleic acids and viral vectors thereof encoding a SARS-CoV-2 spike protein that have been optimized for expression in mammalian cells.

### CROSS-REFERENCE TO RELATED APPLICATIONS

This application claims priority benefit of the filing date of US Provisional Patent Application No. 63/015,352, filed on Apr. 24, 2020, the disclosure of which application is herein incorporated by reference in its entirety.

### BACKGROUND

Coronavirus Disease 19 (COVID-19) is an infectious respiratory illness caused by the severe acute respiratory syndrome coronavirus 2 (SARS-CoV-2). This disease causes fever, severe respiratory illness, and pneumonia. The virus has been characterized as a new member of the betacoronavirus genus, related to several bat coronaviruses and to severe acute respiratory syndrome coronavirus (SARS-CoV). Compared with SARS-CoV, SARS-CoV-2 seems to be more easily transmitted from person to person and has led to a worldwide pandemic. Given the number of variant strains of the SARS-CoV-2 virus, there is a need for methods or systems to evaluate the significance of the variant mutations. There is also a need for a vaccine which can protect against SARS-CoV-2 infection.

### SUMMARY

In certain aspects, provided herein are recombinant nucleic acids, wherein the recombinant nucleic acids encode a SARS-CoV-2 spike protein or a fragment thereof. In some cases, the recombinant nucleic acid comprises a nucleic acid sequence with at least 70% sequence identity to SEQ ID NO: 1 or a portion thereof. In some cases, the nucleic acid sequence comprises at least one silent mutation relative to SEQ ID NO: 1. In some cases, the nucleic acid sequence has at least 75% sequence identity to SEQ ID NO: 1. In some cases, the nucleic acid sequence has at least 80% sequence identity to SEQ ID NO: 1. In some cases, the nucleic acid sequence has at least 85% sequence identity to SEQ ID NO: 1. In some cases, the nucleic acid sequence has at least 90% sequence identity to SEQ ID NO: 1. In some cases, the nucleic acid sequence has at least 95% sequence identity to SEQ ID NO: 1. In some cases, the nucleic acid sequence comprising SEQ ID NO: 1 or a portion thereof comprises at least one mutation present in SEQ ID NO: 3. In some cases, the nucleic acid sequence comprising SEQ ID NO: 1 or a portion thereof comprises at least 3 mutations present in SEQ ID NO: 3. In some cases, the nucleic acid sequence comprising SEQ ID NO: 1 or a portion thereof comprises at least 5 mutations present in SEQ ID NO: 3. In some cases, the nucleic acid sequence comprising SEQ ID NO: 1 or a portion thereof comprises at least 8 mutations present in SEQ ID NO: 3. In some cases, the nucleic acid sequence comprising SEQ ID NO: 1 or a portion thereof comprises at least 12 mutations present in SEQ ID NO: 3. In some cases, the nucleic acid sequence comprising SEQ ID NO: 1 or a portion thereof comprises at least 15 mutations present in SEQ ID NO: 3. In some cases, the nucleic acid sequence comprising SEQ ID NO: 1 or a portion thereof comprises at least 20 mutations present in SEQ ID NO: 3. In some cases, the nucleic acid sequence comprising SEQ ID NO: 1 or a portion thereof comprises at least 25 mutations present in SEQ ID NO: 3. In some cases, the nucleic acid sequence comprising SEQ ID NO: 1 or a portion thereof comprises at least 30 mutations present in SEQ ID NO: 3. In some cases, the nucleic acid sequence comprising SEQ ID NO: 1 or a portion thereof comprises at least 40 mutations present in SEQ ID NO: 3. In some cases, the nucleic acid sequence comprising SEQ ID NO: 1 or a portion thereof comprises at least 50 mutations present in SEQ ID NO: 3. In some cases, the nucleic acid sequence which is codon optimized comprises silent mutations which result in the change of about 25%, about 50%, or about 75% of codons as compared to the wild type sequence. In some cases, the nucleic acid sequence which is codon optimized comprises silent mutations which result in the change of about 25% of codons as compared to the wild type sequence. In some cases, the at least one silent mutation is a codon optimization for transcription in humans. In some cases, the at least one silent mutation reduces the frequency of homologous recombination between the recombinant nucleic acid and chromosomal DNA. In some cases, the at least one silent mutation reduces the frequency of repeat sequences in the recombinant nucleic acid. In some cases, the at least one silent mutation reduces the frequency of homologous recombination between the recombinant nucleic acid and viral DNA. In some cases, the at least one silent mutation reduces the frequency of homologous recombination between the recombinant nucleic acid and human DNA. In some cases, the at least one silent mutation reduces the frequency of homologous recombination between the recombinant nucleic acid and a second recombinant nucleic acid.

In another aspect, there are provided recombinant nucleic acids encoding a SARS-CoV-2 spike protein or a fragment thereof, wherein the recombinant nucleic acid comprises a sequence with at least 70% sequence identity to SEQ ID NO: 3 or a portion thereof. In some cases, the recombinant nucleic acid comprises a sequence with at least 75% sequence identity to SEQ ID NO: 3 or a portion thereof. In some cases, the recombinant nucleic acid comprises a sequence with at least 80% sequence identity to SEQ ID NO: 3 or a portion thereof. In some cases, the recombinant nucleic acid comprises a sequence with at least 85% sequence identity to SEQ ID NO: 3 or a portion thereof. In some cases, the recombinant nucleic acid comprises a sequence with at least 90% sequence identity to SEQ ID NO: 3 or a portion thereof. In some cases, the recombinant nucleic acid comprises a sequence with at least 95% sequence identity to SEQ ID NO: 3 or a portion thereof. In some cases, the recombinant nucleic acid further comprises a sequence encoding a protein tag. In some cases, the protein tag is selected from the group consisting from ALFA-tag, AviTag, C-tag, Calmodulin-tag, polyglutamate tag, E-tag, FLAG-tag, His-tag, Myc-tag, NE-tag, Rho1D4-tag, S-tag, SBP-tag, Softag, HA-tag, Spot-tag, Strep-tag, T7-tag, TC tag, V5 tag, VSV-tag, Xpress tag, biotin carboxyl carrier protein, glutathione-S-transferase, GFP, HaloTag, SNAP-tag, CLIP-tag, HUH-tag, maltose binding protein, and thioredoxin. In some cases, the protein tag comprises a sequence differing from SEQ ID NO: 5 by no more than three point mutations, deletions, or insertions. In some cases, the nucleic acid further comprises at least one sequence selected from the group consisting of a promoter, an enhancer, a viral terminal repeat, a splice site, an origin of replication, a packaging signal, a recombination site, a sequence encoding an epitope recognizable by an antibody, a polyadenylation sequence, a sequence encoding at least one polypeptide, a sequence encoding at least one tRNA sequence, a sequence encoding at least one ribozyme, and a sequence encoding at least one RNA binding protein.

In additional aspects, there are provided viral vectors comprising the recombinant nucleic acid of any of the embodiments herein and a viral genomic sequence. In some cases, the viral vector is replication-deficient. In some cases, the viral vector is a lentiviral vector, a retroviral vector, an adenoviral vector. In some cases, the viral genomic sequence comprises a retrovirus genomic sequence. In some cases, the retrovirus genomic DNA comprises a lentivirus genomic sequence. In some cases, the viral vector comprises a sequence with at least 70% sequence identity to all or a portion of SEQ ID NO: 7. In some cases, the viral vector comprises a sequence with at least 75% sequence identity to all or a portion of SEQ ID NO: 7. In some cases, the viral vector comprises a sequence with at least 80% sequence identity to all or a portion of SEQ ID NO: 7. In some cases, the viral vector comprises a sequence with at least 85% sequence identity to all or a portion of SEQ ID NO: 7. In some cases, the viral vector comprises a sequence with at least 90% sequence identity to all or a portion of SEQ ID NO: 7. In some cases, the viral vector comprises a sequence with at least 95% sequence identity to all or a portion of SEQ ID NO: 7.

In other aspects, the present invention provides methods for evaluating SARS-CoV-2 spike protein variant mutation(s) comprising introducing the SARS-CoV-2 variant mutation(s) into the recombinant nucleic acid sequence of SEQ ID NO: 10, and conducting assays to determine the biological activity of the SARS-CoV-2 variant mutation(s). In certain embodiments, the biological activity comprises cell entry activity or cell fusion.

In other aspects, the present invention provides a pseudotyped lentiviral vector comprising (a) a pBOB-CAG vector, (b) a nucleic acid sequence encoding a SARS-CoV-2 spike protein or a fragment thereof comprising a nucleic acid sequence with at least 90% sequence identity to SEQ ID NO: 9, and (c) a C-terminal HA tag. In some embodiments, the pBOB-CAG vector comprises the nucleic acid sequence set forth in SEQ ID NO: 8. In some embodiments, the nucleic acid sequence encoding a SARS-CoV-2 spike protein or a fragment thereof comprises SEQ ID NO: 9. In certain embodiments, the nucleic acid sequence set forth in SEQ ID NO: 9 encodes a SARS-CoV-2 spike protein or a fragment thereof, wherein the nucleic acid sequences has at least 50%, at least 55%, at least 60%, at least 65%, at least 70% at least 75%, at least 80%, at least 85%, at least 90%, or at least 95% sequence identity to the wild type nucleic acid sequence of SARS-CoV-2 spike protein or a fragment thereof.

In other aspects, the present invention provides a pseudotyped lentiviral vector comprising (a) a pBOB-CAG vector, wherein the pBOB-CAG vector comprises the nucleic acid sequence set forth in SEQ ID NO: 8, (b) a nucleic acid sequence encoding a SARS-CoV-2 spike protein or a fragment thereof comprising a nucleic acid sequence set forth in SEQ ID NO: 9, and (c) a C-terminal HA tag. In certain embodiments, the pseudotyped lentiviral vector of the invention comprises the nucleic acid sequence set forth in SEQ ID NO: 10. In some embodiments, the pseudotyped lentiviral vector of the invention is the nucleic acid sequence set forth in SEQ ID NO: 10.

In further aspects, there are provided recombinant peptides comprising a sequence with at least 70% sequence identity to SEQ ID NO: 2 and a protein tag. In some cases, the recombinant peptide comprises a sequence with at least 75% sequence identity to SEQ ID NO: 2. In some cases, the recombinant peptide comprises a sequence with at least 80% sequence identity to SEQ ID NO: 2. In some cases, the recombinant peptide comprises a sequence with at least 80% sequence identity to SEQ ID NO: 2. In some cases, the recombinant peptide comprises a sequence with at least 85% sequence identity to SEQ ID NO: 2. In some cases, the recombinant peptide comprises a sequence with at least 90% sequence identity to SEQ ID NO: 2. In some cases, the recombinant peptide comprises a sequence with at least 95% sequence identity to SEQ ID NO: 2. In some cases, the protein tag comprises a sequence differing from SEQ ID NO: 6 by no more than two point mutations, deletions, or insertions. In some cases, the recombinant peptide comprises a second protein tag. In some cases, the recombinant peptide has a post-translation modification. In some cases, the post-translational modification is cleavage. In some cases, the post-translational modification comprises a disulfide bond. In some cases, the post-translational modification comprises glycosylation.

In additional aspects, there are provided recombinant peptides comprising a homotrimer, wherein the homotrimer comprises any of the recombinant peptides provided herein.

In further aspects, there are provided recombinant viral particles comprising any recombinant nucleic acid provided herein, any viral vector provided herein, or any recombinant peptide provided herein. In some cases, the recombinant viral particle is selected from the group consisting of a recombinant adenovirus, a recombinant adeno-associated virus, a recombinant myxoma virus, a recombinant Sendai virus, a recombinant measles virus, a recombinant Coxsackie virus, a recombinant Seneca Valley virus, a recombinant Newcastle disease virus, a recombinant vaccinia virus, a recombinant retrovirus, a recombinant herpesvirus, a recombinant pox virus, a recombinant flavivirus, a recombinant togavirus, and a recombinant alphavirus. In some cases, the recombinant viral particle is a recombinant retroviral particle. In some cases, the recombinant viral particle is a recombinant lentiviral particle. In some cases, the recombinant virus is replication deficient. In some cases, the recombinant virus is self-inactivating. In some cases, the recombinant virus is multiply pseudotyped.

In additional aspects, there are provided engineered cells, wherein the engineered cells comprise any recombinant nucleic acid provided herein, any viral vector provided herein, any recombinant peptide provided herein, or any recombinant viral particle provided herein. In some cases, the engineered cell is transfected with any nucleic acid provided herein. In some cases, the transfection is transient transfection. In some cases, the transfection is stable transfection. In some cases, the nucleic acid provided herein is integrated into the genome of the cell. In some cases, the surface of the engineered cell comprises any recombinant peptide provided herein. In some cases, the engineered cell comprises an MHC molecule and a portion of a SARS-CoV-2 spike protein encoded by the nucleic acid provided herein. In some cases, the engineered cell is isolated from an organism. In some cases, the engineered cell is isolated from living tissue. In some cases, the engineered cell is a mammalian cell. In some cases, the mammalian cell is a human cell. In some cases, the human cell is selected from the group consisting of induced pluripotent stem cells, embryonic stem cells, non-embryonic stem cells, amniotic stem cells, lung organoids, dendritic cells, peripheral blood cells, macrophages, T cells, B cells, neutrophils, natural killer cells, natural killer T cells, and leukocytes.

In further aspects, there are provided transgenic animals, wherein the transgenic animal comprises any recombinant nucleic acid provided herein, any viral vector provided herein, any recombinant peptide provided herein, any recombinant viral particle provided herein, or any engineered cell provided herein. In some cases, at least one cell of the transgenic animal is transiently transfected with the nucleic acid provided herein. In some cases, at least one cell of the transgenic animal is stably transfected with the nucleic acid provided herein.

In additional aspects, there are provided therapeutic compositions comprising any recombinant nucleic acid provided herein, any viral vector provided herein, any recombinant peptide provided herein, any recombinant viral particle provided herein, or any engineered cell provided herein. In some cases, the therapeutic composition further comprises an adjuvant. In some cases, the therapeutic composition further comprises a pharmaceutical excipient.

In further aspects, there are provided methods of expressing a transcript encoding SARS-CoV-2 spike. In some cases, methods herein comprise contacting a cell with a nucleic acid provided herein; and culturing the cell to express the transcript. In additional aspects, there are provided methods of expressing a recombinant SARS-CoV-2 spike peptide or a fragment thereof, comprising: contacting a cell with a nucleic acid provided herein; and culturing the cell to express the peptide. In some cases, the SARS-CoV-2 spike peptide is expressed on the cell surface. In some cases, the SARS-CoV-2 spike peptide is expressed in the endoplasmic reticulum. In some cases, the SARS-CoV-2 spike peptide is degraded by the cell and presented on the cell surface. In some cases, the degraded SARS-CoV-2 spike peptide is presented on the cell surface in the context of a MHC molecule. In some cases, the MHC molecule presents an epitope derived from a degraded SARS-CoV-2 spike peptide. In some cases, the nucleic acid encodes a SARS-CoV-2 spike peptide comprising a modification. In some cases, the modification comprises a protein tag selected from HA, FLAG, His, a fluorescent label, GST, V5, MYC, SPOT, T7, OR NE. In some cases, the fluorescent label is GFP, RFP, YFP, CFP, BFP, mBanana, mCherry, and modifications thereof. In some cases, contacting the cell with the nucleic acid comprises incubating the cell with the nucleic acid. In some cases, the nucleic acid is a vector comprising the nucleic acid encoding the SARS-CoV-2 spike peptide. In some cases, the vector is a viral vector. In some cases, the viral vector is a retroviral, adenoviral, adeno-associated viral or lentiviral vector. In some cases, contacting the cell with the nucleic acid comprises contacting the cell with a viral particle comprising the nucleic acid. In some cases, the viral particle is a retroviral particle, adenoviral particle, adeno-associated viral, or lentiviral particle. In some cases, the cell is a fibroblast, iPS cell, CHO, HEK293, HEK293T, HEK293FT, HeLa, Jurkat, Caco-2, High Five, SF-9, U2-OS, BEAS-2B, A549, Calu-3, 16HBE14o-, lung epithelial cell, cardiomyocyte, neuron, PCS-130, or other muscle cell. In some cases, the cell is derived from a subject. In some cases, the cell is a CD4+ T cell, CD8+ T cell, memory cell, natural killer cell, dendritic cell, fibroblast, macrophages, Langerhans cells, B cells, and other antigen presenting cells.

In additional aspects, there are provided methods of vaccinating a subject against SARS-COV-2 infection comprising administering a pharmaceutical composition comprising a cell expressing a transcript encoding a SARS-CoV-2 spike peptide or a fragment thereof according to methods herein. In some cases, the cell is autologous or allogenic to the subject in need thereof. In some cases, the pharmaceutical composition further comprises an adjuvant. In some cases, the adjuvant comprises ABM2, AS01B, AS02, AS02A, Adjumer, Adjuvax, Algammulin, Alum, Aluminum phosphate, Aluminum potassium sulfate, Bordetella pertussis, Calcitriol, CD70, Chitosan, Cholera toxin, CpG, a cytokine, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, or Zymosan. In some cases, the pharmaceutical composition further comprises an excipient. In some cases, the administration of the pharmaceutical composition elicits a T-cell response in the subject in need thereof. In some cases, the administration of the pharmaceutical composition elicits an antibody response in the subject in need thereof.

In further aspects, there are provided methods of vaccinating a subject against SARS-CoV-2 infection comprising administering a pharmaceutical composition comprising a nucleic acid encoding a codon-optimized SARS-CoV-2 spike peptide or a fragment thereof to a subject in need thereof. In some cases, the nucleic acid is a vector. In some cases, the vector is a viral vector. In some cases, the viral vector is a retroviral, adenoviral, adeno-associated viral, or lentiviral vector. In some cases, the pharmaceutical composition further comprises an adjuvant. In some cases, the adjuvant comprises ABM2, AS01B, AS02, AS02A, Adjumer, Adjuvax, Algammulin, Alum, Aluminum phosphate, Aluminum potassium sulfate, Bordetella pertussis, Calcitriol, CD70, Chitosan, Cholera toxin, CpG, a cytokine, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, or Zymosan. In some cases, the pharmaceutical composition further comprises an excipient. In some cases, the administration of the pharmaceutical composition elicits a T-cell response in the subject in need thereof. In some cases, the administration of the pharmaceutical composition elicits an antibody response in the subject in need thereof.

In additional aspects, there are provided methods of vaccinating a subject against SARS-COV-2 infection comprising administering a pharmaceutical composition comprising a modified SARS-CoV-2 spike peptide or a fragment thereof to a subject in need thereof. In some cases, the modified SARS-CoV-2 spike peptide modification comprises a protein tag selected from HA, FLAG, His, a fluorescent label, GST, V5, MYC, SPOT, T7, or NE. In some cases, the fluorescent label is GFP, RFP, YFP, CFP, BFP, mBanana, mCherry, and modifications thereof. In some cases, the pharmaceutical composition further comprises an adjuvant. In some cases, the adjuvant comprises ABM2, AS01B, AS02, AS02A, Adjumer, Adjuvax, Algammulin, Alum, Aluminum phosphate, Aluminum potassium sulfate, Bordetella pertussis, Calcitriol, CD70, Chitosan, Cholera toxin, CpG, a cytokine, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, or Zymosan. In some cases, the pharmaceutical composition further comprises an excipient. In some cases, the administration of the pharmaceutical composition elicits a T-cell response in the subject in need thereof. In some cases, the administration of the pharmaceutical composition elicits an antibody response in the subject in need thereof.

In further aspects, there are provided methods of culturing a cell containing a nucleic acid encoding a SARS-CoV-2 spike peptide comprising: incubating the cell with an effective culture media at a temperature between 20-50 degrees Celsius, wherein culturing the cell (i) produces the SARS-CoV-2 spike peptide; (ii) produces a viral particle comprising the SARS-CoV-2 spike peptide; or (iii) comprises an assay for analysis of the SARS-CoV-2 spike peptide.

In additional aspects, there are provided methods of making an antibody specifically binds to a SARS-CoV-2 spike peptide comprising: exposing a mammal to (i) a nucleic acid that encodes SARS-CoV-2 spike peptide or a fragment thereof, or (ii) SARS-CoV-2 spike peptide, or fragment thereof, in an amount sufficient to produce an antibody; and isolating the antibody from the mammal. In some cases, the antibody is a monoclonal antibody. In some cases, the antibody is an antibody fragment. In some cases, the antibody fragment comprises a Fab, a Fab′, a F(ab′)2, a single-chain Fv (scFv), a Fv, a dsFv, a diabody, a (ds Fv)2), a single chain antibody, a minibody, a flex minibody, or a Fd. In some cases, the antibody is human, humanized, or chimeric.

In further aspects, there are provided method of vaccinating or treating a subject against SARS-COV-2 infection comprising administering a pharmaceutical composition comprising an antibody created by any method provided herein to a subject in need thereof. In some cases, the pharmaceutical composition further comprises an adjuvant. In some cases, the adjuvant comprises ABM2, AS01B, AS02, AS02A, Adjumer, Adjuvax, Algammulin, Alum, Aluminum phosphate, Aluminum potassium sulfate, Bordetella pertussis, Calcitriol, CD70, Chitosan, Cholera toxin, CpG, a cytokine, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, or Zymosan. In some cases, the pharmaceutical composition further comprises an excipient. In some cases, the administration of the pharmaceutical composition elicits a T-cell response in the subject in need thereof.

In further aspects, there are provided methods for producing a transgenic animal comprising contacting a gamete, an embryo, or an animal with any recombinant nucleic acid provided herein, any viral vector provided herein, or any recombinant viral particle provided herein. In some cases, the gamete, embryo, or animal is contacted with the exogenous nucleic acid through nuclear transfer, pro-nuclear injection, viral transduction, transfection, electroporation, or embryonic stem cell implantation. In some cases, the nucleic acid is integrated into a genome of the cell using a Cas nuclease, TALEN, zinc finger nuclease, or Cre recombinase. In some cases, expression of the exogenous nucleic acid is controlled by a tissue-specific promoter. In some cases, the tissue-specific promoter is a lung-specific promoter. In some cases, the lung-specific promoter is SP-B or SP-C. In some cases, the expression of SARS-CoV-2 spike peptide is controlled by a inducible operator system or inducible promoter. In some cases, the inducible operator system is Tet-on or Tet-off.

In additional aspects, there are provided methods for evaluating SARS-CoV-2 spike peptide toxicity comprising exposing a human cell or tissue to a peptide encoded by any recombinant nucleic acid provided herein and detecting a toxicity of the peptide. In some cases, detecting the toxicity of the peptide comprises evaluating cellular viability signals comprising morphological changes, cellular oxidoreductase activity, cytokine activity, cellular esterase activity, oxygen consumption, or a combination thereof. In some cases, cytokine activity comprises levels of IL-6, IL-10, TNF-α, or a combination thereof. In some cases, the SARS-CoV-2 spike peptide is a component of a pseudovirus or virus-like particle. In some cases, the pseudovirus comprises a retroviral, adenoviral, adeno-associated viral, or lentiviral pseudotype. In some cases, the SARS-CoV-2 spike peptide is a component of a cell. In some cases, the human cells or tissues comprise lung epithelial cells, cells from a patient biopsy, patient iPS cells, or patient iPSC-derived lung organoids.

In further aspects, there are provided methods for analyzing SARS-CoV-2 spike peptide glycosylation comprising analyzing isolated SARS-CoV-2 spike peptide for glycosylation, glycosylation patterns, or both. In some cases, SARS-CoV-2 spike peptide is analyzed using a mass spectrometer.

In additional aspects, there are provided methods for observing viral entry comprising: contacting a human cell or tissue with a viral particle comprising a peptide encoded by any recombinant nucleic acid provided herein; and detecting viral entry into the human cell or tissue. In some cases, the method further comprises evaluating viral replication inside the human cell or tissue. In some cases, human cell or tissue comprises a lung epithelial cell, a cell from a patient biopsy, a patient iPS cell, or a patient iPSC-derived lung organoid. In some cases, the SARS-CoV-2 spike peptide is displayed on a pseudovirus or virus-like particle. In some cases, the pseudovirus is a lentiviral, adenoviral, adeno-associated viral, or retroviral pseudotype. In some cases, the pseudovirus or virus-like particle further comprises a detection label. In some cases, the detection label can be monitored as a signal proportional to viral entry. In some cases, the detection label comprises a fluorescent label, protein tag, or nucleic acid. In some cases, the protein tag comprises HA, FLAG, His, florescent protein, GST, V5, MYC, SPOT, T7, OR NE. In some cases, the fluorescent label comprises GFP, RFP, YFP, CFP, BFP, mBanana, mCherry, and modifications thereof. In some cases, viral entry is determined through an assay chosen from one or more of PCR, antibody-affinity, fluorescence-based assay, western blot, flow cytometry, ELISA, or cytopathic effect. In some cases, cytopathic effect comprises one or more of morphological changes, cellular oxidoreductase activity, cytokine activity, cellular esterase activity, or oxygen consumption. In some cases, the method further comprises determining viral entry in the presence of chemical agents. In some cases, chemical agents can be small molecules, large molecules, antibodies, or antibody fragments. In some cases, the viral entry determination results in the identification of a therapeutic agent.

In further aspects, there are provided methods of analyzing changes in gene expression comprising comparing the gene expression profile of a cell or tissue expressing SARS-CoV-2 spike peptide to that of a cell or tissue not expressing SARS-CoV-2 spike peptide. In additional aspects, there are provided methods of analyzing changes gene expression comprising comparing the gene expression profile of a cell or tissue exposed to SARS-CoV-2 spike peptide to that of a cell or tissue not exposed to SARS-CoV-2 spike peptide. In some cases, the SARS-CoV-2 spike peptide is a component of a cell, pseudovirus, or virus-like particle. In some cases, the gene expression profiles are generated through PCR, RNAseq, fluorescent assay, or a combination thereof. In some cases, cytokine expression profiles are compared between the cells or tissues. In some cases, cytokine expression profiles comprise the expression levels of IL-6, IL-10, and TNF-α.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1 illustrates a viral vector expressing the SARS-CoV-2 spike protein.

FIGS. 2A-2C illustrate a nucleic acid sequence alignment of the wildtype SARS-CoV-2 spike protein nucleic acid compared with a codon optimized SARS-CoV-2 spike protein nucleic acid.

FIGS. 3A-3F illustrate spike protein mediated membrane fusion. (FIG. 3A) Immunostaining of methanol permeabilized 293T cells expressing the SARS-CoV2 Spike protein showed localization mostly in the ER (calnexin marker), but significant amounts on the plasma membrane. Non-permeabilized PFA fixed preparations showed significant amounts of Spike protein on the plasma membrane. (FIG. 3B) Spike protein mediated plasma membrane fusion and syncytia formation and ER-ER mediated nuclear membrane fusion in cells co-transfected with hACE2 and SARS-CoV2 Spike. (FIG. 3C) SARS-CoV2 Spike cell fusion assay. 293T cells were transfected with 1. Spike with EGFP or 2. ACE2 and TdTomato. Cells were then mixed and plated. Cotransfection of ACE2 increased the number and size of fused syncytia. Human ACE2 greatly increased cell fusion and hence very large syncytia. (FIG. 3D) FACS analysis of cell fusion experiments. Observe the depletion of the unfused Spike (GFP/FITC-A channel) population as well as the double positive (Spike GFP/ACE2 TdTomato) population due to the formation of large syncytia. (FIG. 3E) FACS quantification of observed GFP+Tdt+double positive population. Introduction of Spike and ACE2 increased fusion. As a result of increased fusion efficiency with human ACE2, depletion of the double positive population was observed due to formation of large syncytia as seen in FIG. 3C. Fusion statistics: RFP+GFP+293T GFP vs Spike WT-GFP p=0.01035, mouse ACE2 GFP vs Spike WT-GFP p=0.00135, human ACE2 GFP vs Spike WT-GFP p=0.01604. (FIG. 3F) Quantification of FACS double positive (fused cells) depletion due to large syncytia loss. Without supplementation of ACE2 there was no loss of fused cells (small syncytia). With mouse ACE2 the difference between Spike 614D and 614G was not significant (p=0.0722), whereas with human ACE2 the increased 614G syncytial depletion (−11%) as a measure of fusion achieved statistical significance (p=0.0185). GFP depletion statistical significance: Human ACE2 GFP vs Spike WT-GFP p=0.013232, Human ACE2 Spike WT-GFP vs Spike D614G-GFP p=0.01879.

FIGS. 4A-4B depict D614G infectivity enhancement in pseudotyped lentiviral vectors. (FIG. 4A) Lentiviral vector pBOB-CAG-GFP was pseudotyped with either WT Spike or D614G Spike, titered and normalized by the level of HIV p24 Gag protein and used to infect control 293T, and 293T cells stably expressing mouse ACE2 or human ACE2. Fluorescence microscopy of GFP (top) and FACS quantification (bottom) are shown. The D614G mutation increased infectivity of Spike ˜5× (p=0.0471) by FACS. Baseline infection of 293T control cells with pseudotyped 614D spike pseudotyped lentiviral vector is set as 1× (FIG. 4B) Immunoblot of partially purified lentiviral preparations shows that incorporation of Spike protein did not appreciably change with the D614G mutation, nor was proteolytic processing of the S2 fragment different. Viral vector nucleocapsid was quantified using a monoclonal antibody against the HIV p24 Gag protein.

FIG. 5 is an additional representation of the viral vector expressing the SARS-CoV-2 spike protein.

### DETAILED DESCRIPTION

Provided herein are compositions and methods useful in developing treatments and vaccines for SARS-CoV-2 infection, along with compositions for treatments and vaccines themselves. Such methods and compositions may produce or utilize the SARS-CoV-2 spike protein, or fragment thereof, as a target, active pharmaceutical ingredient, or reagent for pharmaceutical development or the pharmaceutical itself. In some cases, SARS-CoV-2 spike protein is encoded by a nucleic acid that has been codon optimized for expression in human cells.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, the term “silent mutation” refers to a nucleotide sequence change that does not alter the amino acid sequence of an encoded protein. For example, a silent mutation can be a point mutation that changes a first codon to a second codon that encodes for the same amino acid. A silent mutation can include changes in one or more than one nucleotide.

As used herein, the term “identity” in the context of two or more nucleic acids or polypeptides, the terms “identity”, “sequence identity” and “percent identity” refer to the nucleotide sequences of the nucleic acids or peptide sequences of the polypeptides, and denote the proportions of the sequences that are identical.

As used herein, the term “protein tag” refers to a polypeptide sequence that can be added to the sequence of another polypeptide. Non-limiting examples of protein tags are ALFA-tag, AviTag, C-tag, Calmodulin-tag, polyglutamate tag, E-tag, FLAG-tag, His-tag, Myc-tag, NE-tag, Rho1D4-tag, S-tag, SBP-tag, Softag, Spot-tag, Strep-tag, T7-tag, TC tag, V5 tag, VSV-tag, Xpress tag, biotin carboxyl carrier protein, glutathione-S-transferase, GFP, HaloTag, SNAP-tag, CLIP-tag, HUH-tag, maltose binding protein, and thioredoxin. In some cases, a protein tag affects the subcellular localization of the polypeptide to which it is attached. In some cases, a protein tag has enzymatic activity. In some cases, a protein tag increases or decreases the half-life of the polypeptide to which it is attached. In some cases, a protein tag enables a means for detection (e.g., fluorescence). In some cases, a protein tag enables or enhances one or more methods for purifying the protein to which it is attached. In some cases, a protein tag may be cleavable from the polypeptide to which it is attached.

As used herein, the term “codon optimization” refers to a process used to improve gene expression and increase the translational efficiency of a gene of interest by accommodating codon bias of the host organism.

As used herein, the term “biological activity” refers to any one or more biological properties of a virus or viral fragment (whether present naturally as found in vivo, or provided or enabled by recombinant means). Biological properties include, but are not limited to, binding a receptor, infectious titre, infectivity, membrane fusion, cell entry, cell fusion, switch of receptor, alteration of proteolytic processing patterns, and antibody evasion.

Compositions

In certain aspects, provided herein are recombinant nucleic acids encoding a SARS-CoV-2 spike protein or a fragment thereof. The recombinant nucleic acids encoding a SARS-CoV-2 spike protein or a fragment thereof of the present invention have been optimized for transcription in humans and exhibit increased expression of transgenes compared to other recombinant nucleic acids encoding a SARS-CoV-2 spike protein or a fragment thereof. Accordingly, the recombinant nucleic acids encoding a SARS-CoV-2 spike protein or a fragment thereof of the present invention exhibit advantageous properties for the preparation of a vaccine against SARS-CoV-2. In some cases, recombinant nucleic acids herein comprise a nucleic acid sequence with at least 70% sequence identity to SEQ ID NO: 1 or a portion thereof, wherein the nucleic acid sequence comprises at least one silent mutation relative to SEQ ID NO: 1.

In additional aspects, there are provided recombinant nucleic acids encoding a SARS-CoV-2 spike protein or a fragment thereof, wherein the recombinant nucleic acid comprises a sequence with at least 70% sequence identity to SEQ ID NO: 3 or a portion thereof.

In further aspects, there are provided, viral vectors comprising nucleic acids encoding a SARS-CoV-2 spike protein or a fragment thereof. In some cases, viral vectors herein comprise a nucleic acid sequence with at least 70% sequence identity to SEQ ID NO: 1 or a portion thereof, wherein the nucleic acid sequence comprises at least one silent mutation relative to SEQ ID NO: 1. In some cases, viral vectors herein comprise a recombinant nucleic acid comprises a sequence with at least 70% sequence identity to SEQ ID NO: 3 or a portion thereof.

In additional aspects, there are provided, recombinant peptides comprising a sequence with at least 70% sequence identity to SEQ ID NO: 2. In some cases, the recombinant peptides comprise a protein tag.

In further aspects, there are provided recombinant peptides comprising a homotrimer, wherein the homotrimer comprises recombinant peptides comprising a sequence with at least 70% sequence identity to SEQ ID NO: 2.

In additional aspects, there are provided recombinant viral particles comprising viral vectors described herein which comprise a nucleic acid sequence with at least 70% sequence identity to SEQ ID NO: 1 or a portion thereof, wherein the nucleic acid sequence comprises at least one silent mutation relative to SEQ ID NO: 1. In some cases, viral vectors herein comprise a recombinant nucleic acid comprises a sequence with at least 70% sequence identity to SEQ ID NO: 3 or a portion thereof or recombinant peptides comprising a sequence with at least 70% sequence identity to SEQ ID NO: 2.

In certain aspects, provided herein are recombinant nucleic acids encoding a SARS-CoV-2 spike protein or a fragment thereof. The recombinant nucleic acids encoding a SARS-CoV-2 spike protein or a fragment thereof of the present invention have been optimized for transcription in humans and exhibit increased expression of transgenes compared to other recombinant nucleic acids encoding a SARS-CoV-2 spike protein or a fragment thereof. Accordingly, the recombinant nucleic acids encoding a SARS-CoV-2 spike protein or a fragment thereof of the present invention exhibit advantageous properties for the preparation of a vaccine against SARS-CoV-2. In some cases, recombinant nucleic acids herein comprise a nucleic acid sequence with at least 70% sequence identity to the wild type nucleic acid sequence encoding the SARS-CoV-2 spike protein or a portion thereof, wherein the nucleic acid sequence comprises at least one silent mutation relative to the wild type nucleic acid sequence encoding the SARS-CoV-2 spike protein.

In additional aspects, there are provided recombinant nucleic acids encoding a SARS-CoV-2 spike protein or a fragment thereof, wherein the recombinant nucleic acid comprises a sequence with at least 70%, at least 80%, at least 90%, or at least 95% sequence identity to SEQ ID NO: 9 or a portion thereof. In certain embodiments, the codon optimized SARS CoV-2 spike protein comprises SEQ ID NO: 9.

In further aspects, there are provided viral vectors comprising nucleic acids encoding a SARS-CoV-2 spike protein or a fragment thereof. In some cases, viral vectors herein comprise a nucleic acid sequence with at least 70% sequence identity to SEQ ID NO: 10 or a portion thereof, wherein the nucleic acid sequence comprises at least one silent mutation relative to the wild type nucleic acid sequence encoding the SARS-CoV-2 spike protein. In some cases, viral vectors herein comprise a recombinant nucleic acid comprises a sequence with at least 70% sequence identity to SEQ ID NO: 10 or a portion thereof. In some cases, the viral vector of the invention comprises SEQ ID NO: 10.

In additional aspects, there are provided recombinant viral particles comprising viral vectors described herein. In some cases, viral vectors herein comprise a recombinant nucleic acid comprising a sequence with at least 70% sequence identity to SEQ ID NO: 10 or a portion thereof.

In further aspects, there are provided engineered cells comprising any of the recombinant nucleic acids provided herein, any of the viral vectors provided herein, any of the recombinant peptides provided herein, or any of the recombinant viral particles provided herein.

In additional aspects, there are provided transgenic animals comprising any of the recombinant nucleic acids provided herein, any of the viral vectors provided herein, any of the recombinant peptides provided herein, or any of the recombinant viral particles provided herein.

In further aspects, there are provided therapeutic compositions comprising any of the recombinant nucleic acids provided herein, any of the viral vectors provided herein, any of the recombinant peptides provided herein, or any of the recombinant viral particles provided herein.

### Methods

Provided herein are methods of expressing a transcript encoding SARS-CoV-2 spike protein. In some cases, methods herein comprise contacting a cell with a nucleic acid provided herein; and culturing the cell to express the transcript.

Further provided herein are methods of expressing a recombinant SARS-CoV-2 spike peptide or a fragment thereof. In some cases, methods herein comprise contacting a cell with a nucleic acid provided herein; and culturing the cell to express the peptide.

Additionally provided herein are methods of vaccinating a subject against SARS-CoV-2 infection comprising administering a pharmaceutical composition comprising a cell expressing a SARS-CoV-2 spike peptide provided herein a subject in need thereof.

Further provided herein are methods of vaccinating a subject against SARS-COV-2 infection comprising administering a pharmaceutical composition comprising a nucleic acid encoding a codon-optimized SARS-CoV-2 spike peptide or a fragment thereof to a subject in need thereof.

Additionally provided herein are methods of vaccinating a subject against SARS-COV-2 infection comprising administering a pharmaceutical composition comprising a modified SARS-CoV-2 spike peptide or a fragment thereof to a subject in need thereof.

Further provided herein are methods of culturing a cell containing a nucleic acid encoding a SARS-CoV-2 spike peptide. In some cases, methods comprise: incubating the cell with an effective culture media at a temperature between 20-50 degrees Celsius. In some cases, culturing the cell produces the SARS-CoV-2 spike peptide. In some cases, culturing the cell produces a viral particle comprising the SARS-CoV-2 spike peptide. In some cases, culturing the cell comprises an assay for analysis of the SARS-CoV-2 spike peptide.

Additionally provided herein are methods of making an antibody that targets SARS-CoV-2 spike peptide comprising: exposing a mammal to a nucleic acid that encodes SARS-CoV-2 spike peptide or a fragment thereof, or SARS-CoV-2 spike peptide, or fragment thereof, in an amount sufficient to produce an antibody.

Further provided herein are methods of vaccinating or treating a subject against SARS-COV-2 infection comprising administering a pharmaceutical composition comprising an antibody provided herein to a subject in need thereof.

Additionally provided herein are methods for producing a transgenic animal comprising contacting a gamete, an embryo, or an animal with any recombinant nucleic acid provided herein, an viral vector provided herein, or any recombinant viral particle provided herein.

Further provided herein are methods for evaluating SARS-CoV-2 spike peptide toxicity comprising exposing a human cell or tissue to a peptide encoded by any recombinant nucleic acid provided herein and detecting a toxicity of the peptide.

Additionally provided herein are methods for analyzing SARS-CoV-2 spike peptide glycosylation comprising analyzing isolated SARS-CoV-2 spike peptide for glycosylation, glycosylation patterns, or both.

Further provided herein are methods for observing viral entry comprising contacting a human cell or tissue with a viral particle comprising a peptide encoded by any recombinant nucleic acid provided herein; and detecting viral entry into the cell.

Additionally provided herein are methods of analyzing changes in gene expression comprising comparing the gene expression profile of a cell or tissue expressing SARS-CoV-2 spike peptide to that of a cell or tissue not expressing SARS-CoV-2 spike peptide.

Additionally provided herein are methods for evaluating the significance of SARS-CoV-2 spike protein variant mutations. In one aspect, methods for evaluating the significance of SARS-CoV-2 spike protein variant mutations comprising introducing the SARS-CoV-2 variant mutation into the recombinant nucleic acid sequence of SEQ ID NO: 3; and conducting assays to determine the biological activity of the SARS-CoV-2 variant mutation. In some embodiments, the biological activity is selected from the group consisting of binding a receptor, infectious titre, infectivity, membrane fusion, cell entry, cell fusion, switch of receptor, alteration of proteolytic processing patterns, and antibody evasion. In certain embodiments, the biological activity comprises cell entry activity or cell fusion. Accordingly, the methods provided herein allow for the determination of how specific SARS-CoV-2 mutation(s) alter, e.g., infectivity or the antigenic profile of the virus, thus allowing for a determination of the effectiveness of vaccines and immunotherapeutic treatments. Methods of introducing mutations corresponding to SARS-CoV-2 variant mutations into the lentiviral vector of the invention can be achieved using well known methods. Using the lentiviral vector of the present invention, the wild type and variant(s) can be expressed and assays can be conducted to determine the biological significance of new variant(s). For example, it can be determined using the lentiviral vector of the invention whether new variant(s) have increased infectivity as compared to wild type SARS-CoV-2 or other variant(s).

Further provided herein are methods of analyzing changes in gene expression comprising comparing the gene expression profile of a cell or tissue exposed to SARS-CoV-2 spike peptide to that of a cell or tissue not exposed to SARS-CoV-2 spike peptide.

TABLE 1
Sequences

| SEQ ID NO | DESCRIPTION | SEQUENCE |
|---|---|---|
| 1 | WT CoV-2 DNA | ATTTTGGCAAAGAATTCgCTAGTGGATCcaGGCCGCCTGGGCCCGTTAACA |
| | Sequence encoding | CCATGTTCGTATTCCTTGTCCTCCTCCCCCTCGTCAGCTCCCAGTGTGTCA |
| | S-Protein | ATCTGACAACTCGAACTCAGCTCCCACCCGCATACACCAATAGTTTTACCC |
| | (including regions | GAGGAGTCTATTATCCCGACAAAGTTTTCAGgTCTAGTGTGTTGCATAGCA |
| | upstream and | CGCAGGACCTGTTCTTGCCGTTTTTTTCAAATGTGACATGGTTCCATGCCA |
| | downstream ORF) | TCCATGTCTCTGGTACGAACGGGACCAAAAGGTTTGATAACCCCGTTCTTC |
| | | CATTTAACGACGGGGTTTATTTCGCCTCTACTGAAAAGTCTAATATAATAC |
| | | GAGGTTGGATTTTTGGGACAACACTGGACTCTAAAACGCAATCTCTCCTCA |
| | | TAGTAAATAATGCGACGAATGTCGTAATCAAAGTATGTGAGTTTCAGTTTT |
| | | GCAACGACCCTTTTCTTGGTGTATACTATCACAAGAACAACAAGTCTTGGA |
| | | TGGAAAGTGAATTtAGAGTCTACAGCTCCGCAAACAACTGTACCTTTGAGT |
| | | ACGTCAGTCAGCCCTTCCTTATGGACCTCGAAGGCAAACAGGGGAACTTCA |
| | | AGAATCTGCGCGAGTTCGTGTTCAAGAATATCGACGGCTATTTCAAGATAT |
| | | ATTCAAAACATACACCAATCAATTTGGTACGCGATTTGCCGCAGGGCTTCT |
| | | CTGCTCTTGAGCCTCTCGTAGATTTGCCTATAGGGATCAATATTACTCGCT |
| | | TTCAAACTCTCCTTGCCCTTCATCGGTCCTACCTGACGCCAGGCGACAGCT |
| | | CCAGTGGGTGGACAGCTGGTGCAGCGGCTTACTACGTCGGCTACCTCCAAC |
| | | CCAGGACTTTTTTGCTGAAATATAATGAGAATGGTACGATTACTGACGCCG |
| | | TCGATTGTGCCCTCGACCCATTGAGCGAAACTAAATGCACCCTGAAAAGCT |
| | | TTACGGTTGAGAAAGGGATATATCAGACATCTAATTTTCGCGTTCAACCCA |
| | | CGGAATCCATTGTACGATTTCCCAACATTACCAATTTGTGTCCTTTTGGTG |
| | | AGGTATTTAATGCAACCAGATTCGCGAGTGTGTATGCCTGGAATAGGAAAA |
| | | GAATTTCTAACTGTGTAGCAGACTACAGCGTTCTGTATAATAGTGCGTCCT |
| | | TTAGTACGTTCAAGTGCTACGGAGTAAGCCCGACAAAGTTGAACGATTTGT |
| | | GTTTCACTAACGTCTATGCCGATAGCTTCGTCATCAGGGGAGACGAAGTAA |
| | | GGCAAATTGCACCAGGCCAGACCGGaAAAATAGCCGACTACAACTATAAGT |
| | | TGCCGGACGATTTCACCGGGTGTGTCATCGCGTGGAATAGTAATAACCTGG |
| | | ACTCCAAGGTGGGCGGCAACTACAACTACCTGTACCGATTGTTCCGCAAGT |
| | | CTAACCTCAAGCCCTTTGAACGAGACATATCCACCGAGATATACCAAGCGG |
| | | GAAGCACTCCCTGTAATGGCGTTGAAGGTTTCAACTGCTATTTTCCTCTCC |
| | | AGTCCTATGGCTTCCAACCTACCAATGGAGTAGGCTATCAACCGTATCGAG |
| | | TCGTGGTGCTGAGTTTCGAGCTCCTGCACGCTCCAGCGACCGTTTGTGGTC |
| | | CTAAGAAAAGTACAAACCTCGTAAAAAATAAGTGCGTAAACTTTAACTTTA |
| | | ACGGTCTCACAGGAACGGGAGTTCTTACAGAGTCCAATAAGAAGTTCCTGC |
| | | CCTTTCAGCAGTTTGGGCGCGACATCGCGGACACCACAGATGCAGTCAGGG |
| | | ACCCGCAGACTCTTGAGATCCTGGAcATCACCCCATGCTCTTTTGGCGGTG |
| | | TGAGTGTCATTACACCCGGGACCAACACCAGCAATCAGGTTGCTGTGCTCT |
| | | ATCAAGACGTCAACTGTACGGAAGTCCCAGTTGCAATACACGCGGACCAGT |
| | | TGACGCCGACGTGGCGGGTTTATTCTACCGGCTCAAACGTCTTTCAAACTA |
| | | GAGCCGGTTGTTTGATCGGGGCGGAGCATGTGAACAATTCATACGAATGTG |
| | | ATATTCCCATAGGAGCCGGAATATGTGCTTCTTACCAGACCCAAACCAACT |
| | | CTCCGAGACGAGCCCGGTCCGTAGCCAGTCAAAGCATAATTGCGTACACCA |
| | | TGAGCCTCGGTGCAGAAAACTCAGTTGCGTACTCAAATAACTCCATTGCCA |
| | | TCCCAACAAATTTTACCATATCAGTGACCACTGAAATCCTTCCAGTGAGCA |
| | | TGACTAAAACAAGTGTAGATTGCACAATGTAtATTTGCGGAGACTCAACTG |
| | | AGTGCTCTAACCTCCTGTTGCAGTACGGTTCATTCTGTACCCAGCTCAATC |
| | | GCGCACTTACGGGAATAGCAGTAGAACAAGATAAGAATACTCAGGAAGTCT |
| | | TCGCTCAAGTAAAGCAAATCTACAAGACGCCCCCCATTAAGGACTTTGGGG |
| | | GTTTTAATTTTAGCCAAATACTCCCGGAcCCCAGTAAACCCTCTAAGAGGT |
| | | CATTTATAGAAGACCTGTTGTTTAATAAAGTTACTCTGGCCGATGCTGGCT |
| | | TTATTAAACAATACGGTGATTGTTTGGGCGACATCGCGGCGCGGGATCTGA |
| | | TATGCGCCCAAAAATTTAACGGTCTCACTGTGTTGCCGCCTTTGCTCACAG |
| | | ACGAGATGATAGCTCAGTACACATCAGCGTTGTTGGCGGGTACCATAACGT |
| | | CtGGATGGACATTCGGAGCAGGGGCGGCCTTGCAAATACCATTCGCAATGC |
| | | AAATGGCATACAGGTTCAACGGGATAGGTGTTACACAGAATGTACTTTACG |
| | | AGAACCAGAAATTGATTGCAAACCAGTTTAACTCTGCGATTGGCAAAATTC |
| | | AGGATAGCCTCAGCTCCACCGCGTCTGCACTGGGAAAACTCCAAGATGTTG |
| | | TgAACCAAAATGCCCAAGCCCTCAATACACTTGTGAAGCAACTCTCCAGCA |
| | | ATTTCGGTGCAATAAGTAGTGTGCTGAACGACATACTTTCTCGGTTGGACA |
| | | AAGTAGAGGCGGAGGTGCAAATCGACAGACTCATAACAGGTCGACTGCAAT |
| | | CATTGCAAACGTATGTTACTCAGCAATTGATACGGGCGGCGGAGATTCGAG |
| | | CGTCTGCCAACCTCGCCGCGACTAAAATGTCTGAGTGTGTGCTCGGTCAGT |
| | | CAAAACGCGTGGACTTCTGTGGAAAGGGATACCATCTCATGTCCTTCCCTC |
| | | AGTCTGCCCCTCACGGAGTAGTCTTTCTTCAtGTGACCTATGTTCCAGCAC |
| | | AGGAGAAAAATTTTACCACAGCGCCCGCGATATGCCACGATGGCAAAGCTC |
| | | ATTTTCCGCGAGAGGGAGTATTTGTCTCTAACGGCACCCATTGGTTTGTAA |
| | | CCCAGCGCAATTTTTACGAACCTCAGATAATCACCACTGACAACACCTTTG |
| | | TTAGTGGTAATTGTGATGTAGTGATCGGGATAGTgAACAATACAGTGTACG |
| | | ACCCCCTGCAACCTGAGCTGGACTCCTTTAAAGAAGAACTCGACAAGTACT |
| | | TCAAAAACCACACTTCCCCGGATGTCGATTTGGGTGATATATCTGGCATCA |
| | | ATGCGAGTGTAGTCAACATACAGAAGGAAATCGACAGGCTCAATGAAGTAG |
| | | CAAAAAACCTGAATGAATCCCTCATAGAcCTCCAAGAGCTGGGCAAATATG |
| | | AGCAGTACATTAAATGGCCCTGGTACATTTGGCTGGGCTTCATCGCTGGCC |
| | | TGATAGCGATAGTGATGGTTACTATTATGCTCTGCTGTATGACGAGTTGTT |
| | | GCAGCTGCCTCAAAGGGTGTTGTTCTTGCGGcTCCTGTTGTAAGTTCGACG |
| | | AGGATGATTCAGAGCCCGTTCTTAAGGGTGTAAAACTCCATTACACTGGAG |
| | | GAGGAGGTTACCCATACGATGTTCCAGATTACGCTTAGTAAACTAGTAACG |
| | | TCTCGAGTTTAAACCTGGAAAAACATGGAG |
| 2 | WT CoV-2 S- | MFVFLVLLPLVSSQCVNLTTRTQLPPAYTNSFTRGVYYPDKVFRSSVLHST |
| | Protein Amino | QDLFLPFFSNVTWFHAIHVSGTNGTKRFDNPVLPFNDGVYFASTEKSNIIR |
| | Acid Sequence | GWIFGTTLDSKTQSLLIVNNATNVVIKVCEFQFCNDPFLGVYYHKNNKSWM |
| | | ESEFRVYSSANNCTFEYVSQPFLMDLEGKQGNFKNLREFVFKNIDGYFKIY |
| | | SKHTPINLVRDLPQGFSALEPLVDLPIGINITRFQTLLALHRSYLTPGDSS |
| | | SGWTAGAAAYYVGYLQPRTFLLKYNENGTITDAVDCALDPLSETKCTLKSF |
| | | TVEKGIYQTSNFRVQPTESIVRFPNITNLCPFGEVFNATRFASVYAWNRKR |
| | | ISNCVADYSVLYNSASFSTFKCYGVSPTKLNDLCFTNVYADSFVIRGDEVR |
| | | QIAPGQTGKIADYNYKLPDDFTGCVIAWNSNNLDSKVGGNYNYLYRLFRKS |
| | | NLKPFERDISTEIYQAGSTPCNGVEGFNCYFPLQSYGFQPTNGVGYQPYRV |
| | | VVLSFELLHAPATVCGPKKSTNLVKNKCVNFNFNGLTGTGVLTESNKKFLP |
| | | FQQFGRDIADTTDAVRDPQTLEILDITPCSFGGVSVITPGTNTSNQVAVLY |
| | | QDVNCTEVPVAIHADQLTPTWRVYSTGSNVFQTRAGCLIGAEHVNNSYECD |
| | | IPIGAGICASYQTQTNSPRRARSVASQSIIAYTMSLGAENSVAYSNNSIAI |
| | | PTNFTISVTTEILPVSMTKTSVDCTMYICGDSTECSNLLLQYGSFCTQLNR |
| | | ALTGIAVEQDKNTQEVFAQVKQIYKTPPIKDFGGFNFSQILPDPSKPSKRS |
| | | HEDLLFNKVTLADAGFIKQYGDCLGDIAARDLICAQKFNGLTVLPPLLTDE |
| | | MIAQYTSALLAGTITSGWTFGAGAALQIPFAMQMAYRFNGIGVTQNVLYEN |
| | | QKLIANQFNSAIGKIQDSLSSTASALGKLQDVVNQNAQALNTLVKQLSSNF |
| | | GAISSVLNDILSRLDKVEAEVQIDRLITGRLQSLQTYVTQQLIRAAEIRAS |
| | | ANLAATKMSECVLGQSKRVDFCGKGYHLMSFPQSAPHGVVFLHVTYVPAQE |
| | | KNFTTAPAICHDGKAHFPREGVFVSNGTHWFVTQRNFYEPQIITTDNTFVS |
| | | GNCDVVIGIVNNTVYDPLQPELDSFKEELDKYFKNHTSPDVDLGDISGINA |
| | | SVVNIQKEIDRLNEVAKNLNESLIDLQELGKYEQYIKWPWYIWLGFIAGLI |
| | | AIVMVTIMLCCMTSCCSCLKGCCSCGSCCKFDEDDSEPVLKGVKLHYT |
| 3 | Vector with codon | TTATACCTTCCCAGGTAACAAACCAACCAACTTTCGATCTCTTGTAGATCT |
| | optimized CoV-2 | GTTCTCTAAACGAACTTTAAAATCTGTGTGGCTGTCACTCGGCTGCATGCT |
| | S-Protein ORF | TAGTGCACTCACGCAGTATAATTAATAACTAATTACTGTCGTTGACAGGAC |
| | | ACGAGTAACTCGTCTATCTTCTGCAGGCTGCTTACGGTTTCGTCCGTGTTG |
| | | CAGCCGATCATCAGCACATCTAGGTTTCGTCCGGGTGTGACCGAAAGGTAA |
| | | GATGGAGAGCCTTGTCCCTGGTTTCAACGAGAAAACACACGTCCAACTCAG |
| | | TTTGCCTGTTTTACAGGTTCGCGACGTGCTCGTACGTGGCTTTGGAGACTC |
| | | CGTGGAGGAGGTCTTATCAGAGGCACGTCAACATCTTAAAGATGGCACTTG |
| | | TGGCTTAGTAGAAGTTGAAAAAGGCGTTTTGCCTCAACTTGAACAGCCCTA |
| | | TGTGTTCATCAAACGTTCGGATGCTCGAACTGCACCTCATGGTCATGTTAT |
| | | GGTTGAGCTGGTAGCAGAACTCGAAGGCATTCAGTACGGTCGTAGTGGTGA |
| | | GACACTTGGTGTCCTTGTCCCTCATGTGGGCGAAATACCAGTGGCTTACCG |
| | | CAAGGTTCTTCTTCGTAAGAACGGTAATAAAGGAGCTGGTGGCCATAGTTA |
| | | CGGCGCCGATCTAAAGTCATTTGACTTAGGCGACGAGCTTGGCACTGATCC |
| | | TTATGAAGATTTTCAAGAAAACTGGAACACTAAACATAGCAGTGGTGTTAC |
| | | CCGTGAACTCATGCGTGAGCTTAACGGAGGGGCATACACTCGCTATGTCGA |
| | | TAACAACTTCTGTGGCCCTGATGGCTACCCTCTTGAGTGCATTAAAGACCT |
| | | TCTAGCACGTGCTGGTAAAGCTTCATGCACTTTGTCCGAACAACTGGACTT |
| | | TATTGACACTAAGAGGGGTGTATACTGCTGCCGTGAACATGAGCATGAAAT |
| | | TGCTTGGTACACGGAACGTTCTGAAAAGAGCTATGAATTGCAGACACCTTT |
| | | TGAAATTAAATTGGCAAAGAAATTTGACACCTTCAATGGGGAATGTCCAAA |
| | | TTTTGTATTTCCCTTAAATTCCATAATCAAGACTATTCAACCAAGGGTTGA |
| | | AAAGAAAAAGCTTGATGGCTTTATGGGTAGAATTCGATCTGTCTATCCAGT |
| | | TGCGTCACCAAATGAATGCAACCAAATGTGCCTTTCAACTCTCATGAAGTG |
| | | TGATCATTGTGGTGAAACTTCATGGCAGACGGGCGATTTTGTTAAAGCCAC |
| | | TTGCGAATTTTGTGGCACTGAGAATTTGACTAAAGAAGGTGCCACTACTTG |
| | | TGGTTACTTACCCCAAAATGCTGTTGTTAAAATTTATTGTCCAGCATGTCA |
| | | CAATTCAGAAGTAGGACCTGAGCATAGTCTTGCCGAATACCATAATGAATC |
| | | TGGCTTGAAAACCATTCTTCGTAAGGGTGGTCGCACTATTGCCTTTGGAGG |
| | | CTGTGTGTTCTCTTATGTTGGTTGCCATAACAAGTGTGCCTATTGGGTTCC |
| | | ACGTGCTAGCGCTAACATAGGTTGTAACCATACAGGTGTTGTTGGAGAAGG |
| | | TTCCGAAGGTCTTAATGACAACCTTCTTGAAATACTCCAAAAAGAGAAAGT |
| | | CAACATCAATATTGTTGGTGACTTTAAACTTAATGAAGAGATCGCCATTAT |
| | | TTTGGCATCTTTTTCTGCTTCCACAAGTGCTTTTGTGGAAACTGTGAAAGG |
| | | TTTGGATTATAAAGCATTCAAACAAATTGTTGAATCCTGTGGTAATTTTAA |
| | | AGTTACAAAAGGAAAAGCTAAAAAAGGTGCCTGGAATATTGGTGAACAGAA |
| | | ATCAATACTGAGTCCTCTTTATGCATTTGCATCAGAGGCTGCTCGTGTTGT |
| | | ACGATCAATTTTCTCCCGCACTCTTGAAACTGCTCAAAATTCTGTGCGTGT |
| | | TTTACAGAAGGCCGCTATAACAATACTAGATGGAATTTCACAGTATTCACT |
| | | GAGACTCATTGATGCTATGATGTTCACATCTGATTTGGCTACTAACAATCT |
| | | AGTTGTAATGGCCTACATTACAGGTGGTGTTGTTCAGTTGACTTCGCAGTG |
| | | GCTAACTAACATCTTTGGCACTGTTTATGAAAAACTCAAACCCGTCCTTGA |
| | | TTGGCTTGAAGAGAAGTTTAAGGAAGGTGTAGAGTTTCTTAGAGACGGTTG |
| | | GGAAATTGTTAAATTTATCTCAACCTGTGCTTGTGAAATTGTCGGTGGACA |
| | | AATTGTCACCTGTGCAAAGGAAATTAAGGAGAGTGTTCAGACATTCTTTAA |
| | | GCTTGTAAATAAATTTTTGGCTTTGTGTGCTGACTCTATCATTATTGGTGG |
| | | AGCTAAACTTAAAGCCTTGAATTTAGGTGAAACATTTGTCACGCACTCAAA |
| | | GGGATTGTACAGAAAGTGTGTTAAATCCAGAGAAGAAACTGGCCTACTCAT |
| | | GCCTCTAAAAGCCCCAAAAGAAATTATCTTCTTAGAGGGAGAAACACTTCC |
| | | CACAGAAGTGTTAACAGAGGAAGTTGTCTTGAAAACTGGTGATTTACAACC |
| | | ATTAGAACAACCTACTAGTGAAGCTGTTGAAGCTCCATTGGTTGGTACACC |
| | | AGTTTGTATTAACGGGCTTATGTTGCTCGAAATCAAAGACACAGAAAAGTA |
| | | CTGTGCCCTTGCACCTAATATGATGGTAACAAACAATACCTTCACACTCAA |
| | | AGGCGGTGCACCAACAAAGGTTACTTTTGGTGATGACACTGTGATAGAAGT |
| | | GCAAGGTTACAAGAGTGTGAATATCACTTTTGAACTTGATGAAAGGATTGA |
| | | TAAAGTACTTAATGAGAAGTGCTCTGCCTATACAGTTGAACTCGGTACAGA |
| | | AGTAAATGAGTTCGCCTGTGTTGTGGCAGATGCTGTCATAAAAACTTTGCA |
| | | ACCAGTATCTGAATTACTTACACCACTGGGCATTGATTTAGATGAGTGGAG |
| | | TATGGCTACATACTACTTATTTGATGAGTCTGGTGAGTTTAAATTGGCTTC |
| | | ACATATGTATTGTTCTTTCTACCCTCCAGATGAGGATGAAGAAGAAGGTGA |
| | | TTGTGAAGAAGAAGAGTTTGAGCCATCAACTCAATATGAGTATGGTACTGA |
| | | AGATGATTACCAAGGTAAACCTTTGGAATTTGGTGCCACTTCTGCTGCTCT |
| | | TCAACCTGAAGAAGAGCAAGAAGAAGATTGGTTAGATGATGATAGTCAACA |
| | | AACTGTTGGTCAACAAGACGGCAGTGAGGACAATCAGACAACTACTATTCA |
| | | AACAATTGTTGAGGTTCAACCTCAATTAGAGATGGAACTTACACCAGTTGT |
| | | TCAGACTATTGAAGTGAATAGTTTTAGTGGTTATTTAAAACTTACTGACAA |
| | | TGTATACATTAAAAATGCAGACATTGTGGAAGAAGCTAAAAAGGTAAAACC |
| | | AACAGTGGTTGTTAATGCAGCCAATGTTTACCTTAAACATGGAGGAGGTGT |
| | | TGCAGGAGCCTTAAATAAGGCTACTAACAATGCCATGCAAGTTGAATCTGA |
| | | TGATTACATAGCTACTAATGGACCACTTAAAGTGGGTGGTAGTTGTGTTTT |
| | | AAGCGGACACAATCTTGCTAAACACTGTCTTCATGTTGTCGGCCCAAATGT |
| | | TAACAAAGGTGAAGACATTCAACTTCTTAAGAGTGCTTATGAAAATTTTAA |
| | | TCAGCACGAAGTTCTACTTGCACCATTATTATCAGCTGGTATTTTTGGTGC |
| | | TGACCCTATACATTCTTTAAGAGTTTGTGTAGATACTGTTCGCACAAATGT |
| | | CTACTTAGCTGTCTTTGATAAAAATCTCTATGACAAACTTGTTTCAAGCTT |
| | | TTTGGAAATGAAGAGTGAAAAGCAAGTTGAACAAAAGATCGCTGAGATTCC |
| | | TAAAGAGGAAGTTAAGCCATTTATAACTGAAAGTAAACCTTCAGTTGAACA |
| | | GAGAAAACAAGATGATAAGAAAATCAAAGCTTGTGTTGAAGAAGTTACAAC |
| | | AACTCTGGAAGAAACTAAGTTCCTCACAGAAAACTTGTTACTTTATATTGA |
| | | CATTAATGGCAATCTTCATCCAGATTCTGCCACTCTTGTTAGTGACATTGA |
| | | CATCACTTTCTTAAAGAAAGATGCTCCATATATAGTGGGTGATGTTGTTCA |
| | | AGAGGGTGTTTTAACTGCTGTGGTTATACCTACTAAAAAGGCTGGTGGCAC |
| | | TACTGAAATGCTAGCGAAAGCTTTGAGAAAAGTGCCAACAGACAATTATAT |
| | | AACCACTTACCCGGGTCAGGGTTTAAATGGTTACACTGTAGAGGAGGCAAA |
| | | GACAGTGCTTAAAAAGTGTAAAAGTGCCTTTTACATTCTACCATCTATTAT |
| | | CTCTAATGAGAAGCAAGAAATTCTTGGAACTGTTTCTTGGAATTTGCGAGA |
| | | AATGCTTGCACATGCAGAAGAAACACGCAAATTAATGCCTGTCTGTGTGGA |
| | | AACTAAAGCCATAGTTTCAACTATACAGCGTAAATATAAGGGTATTAAAAT |
| | | ACAAGAGGGTGTGGTTGATTATGGTGCTAGATTTTACTTTTACACCAGTAA |
| | | AACAACTGTAGCGTCACTTATCAACACACTTAACGATCTAAATGAAACTCT |
| | | TGTTACAATGCCACTTGGCTATGTAACACATGGCTTAAATTTGGAAGAAGC |
| | | TGCTCGGTATATGAGATCTCTCAAAGTGCCAGCTACAGTTTCTGTTTCTTC |
| | | ACCTGATGCTGTTACAGCGTATAATGGTTATCTTACTTCTTCTTCTAAAAC |
| | | ACCTGAAGAACATTTTATTGAAACCATCTCACTTGCTGGTTCCTATAAAGA |
| | | TTGGTCCTATTCTGGACAATCTACACAACTAGGTATAGAATTTCTTAAGAG |
| | | AGGTGATAAAAGTGTATATTACACTAGTAATCCTACCACATTCCACCTAGA |
| | | TGGTGAAGTTATCACCTTTGACAATCTTAAGACACTTCTTTCTTTGAGAGA |
| | | AGTGAGGACTATTAAGGTGTTTACAACAGTAGACAACATTAACCTCCACAC |
| | | GCAAGTTGTGGACATGTCAATGACATATGGACAACAGTTTGGTCCAACTTA |
| | | TTTGGATGGAGCTGATGTTACTAAAATAAAACCTCATAATTCACATGAAGG |
| | | TAAAACATTTTATGTTTTACCTAATGATGACACTCTACGTGTTGAGGCTTT |
| | | TGAGTACTACCACACAACTGATCCTAGTTTTCTGGGTAGGTACATGTCAGC |
| | | ATTAAATCACACTAAAAAGTGGAAATACCCACAAGTTAATGGTTTAACTTC |
| | | TATTAAATGGGCAGATAACAACTGTTATCTTGCCACTGCATTGTTAACACT |
| | | CCAACAAATAGAGTTGAAGTTTAATCCACCTGCTCTACAAGATGCTTATTA |
| | | CAGAGCAAGGGCTGGTGAAGCTGCTAACTTTTGTGCACTTATCTTAGCCTA |
| | | CTGTAATAAGACAGTAGGTGAGTTAGGTGATGTTAGAGAAACAATGAGTTA |
| | | CTTGTTTCAACATGCCAATTTAGATTCTTGCAAAAGAGTCTTGAACGTGGT |
| | | GTGTAAAACTTGTGGACAACAGCAGACAACCCTTAAGGGTGTAGAAGCTGT |
| | | TATGTACATGGGCACACTTTCTTATGAACAATTTAAGAAAGGTGTTCAGAT |
| | | ACCTTGTACGTGTGGTAAACAAGCTACAAAATATCTAGTACAACAGGAGTC |
| | | ACCTTTTGTTATGATGTCAGCACCACCTGCTCAGTATGAACTTAAGCATGG |
| | | TACATTTACTTGTGCTAGTGAGTACACTGGTAATTACCAGTGTGGTCACTA |
| | | TAAACATATAACTTCTAAAGAAACTTTGTATTGCATAGACGGTGCTTTACT |
| | | TACAAAGTCCTCAGAATACAAAGGTCCTATTACGGATGTTTTCTACAAAGA |
| | | AAACAGTTACACAACAACCATAAAACCAGTTACTTATAAATTGGATGGTGT |
| | | TGTTTGTACAGAAATTGACCCTAAGTTGGACAATTATTATAAGAAAGACAA |
| | | TTCTTATTTCACAGAGCAACCAATTGATCTTGTACCAAACCAACCATATCC |
| | | AAACGCAAGCTTCGATAATTTTAAGTTTGTATGTGATAATATCAAATTTGC |
| | | TGATGATTTAAACCAGTTAACTGGTTATAAGAAACCTGCTTCAAGAGAGCT |
| | | TAAAGTTACATTTTTCCCTGACTTAAATGGTGATGTGGTGGCTATTGATTA |
| | | TAAACACTACACACCCTCTTTTAAGAAAGGAGCTAAATTGTTACATAAACC |
| | | TATTGTTTGGCATGTTAACAATGCAACTAATAAAGCCACGTATAAACCAAA |
| | | TACCTGGTGTATACGTTGTCTTTGGAGCACAAAACCAGTTGAAACATCAAA |
| | | TTCGTTTGATGTACTGAAGTCAGAGGACGCGCAGGGAATGGATAATCTTGC |
| | | CTGCGAAGATCTAAAACCAGTCTCTGAAGAAGTAGTGGAAAATCCTACCAT |
| | | ACAGAAAGACGTTCTTGAGTGTAATGTGAAAACTACCGAAGTTGTAGGAGA |
| | | CATTATACTTAAACCAGCAAATAATAGTTTAAAAATTACAGAAGAGGTTGG |
| | | CCACACAGATCTAATGGCTGCTTATGTAGACAATTCTAGTCTTACTATTAA |
| | | GAAACCTAATGAATTATCTAGAGTATTAGGTTTGAAAACCCTTGCTACTCA |
| | | TGGTTTAGCTGCTGTTAATAGTGTCCCTTGGGATACTATAGCTAATTATGC |
| | | TAAGCCTTTTCTTAACAAAGTTGTTAGTACAACTACTAACATAGTTACACG |
| | | GTGTTTAAACCGTGTTTGTACTAATTATATGCCTTATTTCTTTACTTTATT |
| | | GCTACAATTGTGTACTTTTACTAGAAGTACAAATTCTAGAATTAAAGCATC |
| | | TATGCCGACTACTATAGCAAAGAATACTGTTAAGAGTGTCGGTAAATTTTG |
| | | TCTAGAGGCTTCATTTAATTATTTGAAGTCACCTAATTTTTCTAAACTGAT |
| | | AAATATTATAATTTGGTTTTTACTATTAAGTGTTTGCCTAGGTTCTTTAAT |
| | | CTACTCAACCGCTGCTTTAGGTGTTTTAATGTCTAATTTAGGCATGCCTTC |
| | | TTACTGTACTGGTTACAGAGAAGGCTATTTGAACTCTACTAATGTCACTAT |
| | | TGCAACCTACTGTACTGGTTCTATACCTTGTAGTGTTTGTCTTAGTGGTTT |
| | | AGATTCTTTAGACACCTATCCTTCTTTAGAAACTATACAAATTACCATTTC |
| | | ATCTTTTAAATGGGATTTAACTGCTTTTGGCTTAGTTGCAGAGTGGTTTTT |
| | | GGCATATATTCTTTTCACTAGGTTTTTCTATGTACTTGGATTGGCTGCAAT |
| | | CATGCAATTGTTTTTCAGCTATTTTGCAGTACATTTTATTAGTAATTCTTG |
| | | GCTTATGTGGTTAATAATTAATCTTGTACAAATGGCCCCGATTTCAGCTAT |
| | | GGTTAGAATGTACATCTTCTTTGCATCATTTTATTATGTATGGAAAAGTTA |
| | | TGTGCATGTTGTAGACGGTTGTAATTCATCAACTTGTATGATGTGTTACAA |
| | | ACGTAATAGAGCAACAAGAGTCGAATGTACAACTATTGTTAATGGTGTTAG |
| | | AAGGTCCTTTTATGTCTATGCTAATGGAGGTAAAGGCTTTTGCAAACTACA |
| | | CAATTGGAATTGTGTTAATTGTGATACATTCTGTGCTGGTAGTACATTTAT |
| | | TAGTGATGAAGTTGCGAGAGACTTGTCACTACAGTTTAAAAGACCAATAAA |
| | | TCCTACTGACCAGTCTTCTTACATCGTTGATAGTGTTACAGTGAAGAATGG |
| | | TTCCATCCATCTTTACTTTGATAAAGCTGGTCAAAAGACTTATGAAAGACA |
| | | TTCTCTCTCTCATTTTGTTAACTTAGACAACCTGAGAGCTAATAACACTAA |
| | | AGGTTCATTGCCTATTAATGTTATAGTTTTTGATGGTAAATCAAAATGTGA |
| | | AGAATCATCTGCAAAATCAGCGTCTGTTTACTACAGTCAGCTTATGTGTCA |
| | | ACCTATACTGTTACTAGATCAGGCATTAGTGTCTGATGTTGGTGATAGTGC |
| | | GGAAGTTGCAGTTAAAATGTTTGATGCTTACGTTAATACGTTTTCATCAAC |
| | | TTTTAACGTACCAATGGAAAAACTCAAAACACTAGTTGCAACTGCAGAAGC |
| | | TGAACTTGCAAAGAATGTGTCCTTAGACAATGTCTTATCTACTTTTATTTC |
| | | AGCAGCTCGGCAAGGGTTTGTTGATTCAGATGTAGAAACTAAAGATGTTGT |
| | | TGAATGTCTTAAATTGTCACATCAATCTGACATAGAAGTTACTGGCGATAG |
| | | TTGTAATAACTATATGCTCACCTATAACAAAGTTGAAAACATGACACCCCG |
| | | TGACCTTGGTGCTTGTATTGACTGTAGTGCGCGTCATATTAATGCGCAGGT |
| | | AGCAAAAAGTCACAACATTGCTTTGATATGGAACGTTAAAGATTTCATGTC |
| | | ATTGTCTGAACAACTACGAAAACAAATACGTAGTGCTGCTAAAAAGAATAA |
| | | CTTACCTTTTAAGTTGACATGTGCAACTACTAGACAAGTTGTTAATGTTGT |
| | | AACAACAAAGATAGCACTTAAGGGTGGTAAAATTGTTAATAATTGGTTGAA |
| | | GCAGTTAATTAAAGTTACACTTGTGTTCCTTTTTGTTGCTGCTATTTTCTA |
| | | TTTAATAACACCTGTTCATGTCATGTCTAAACATACTGACTTTTCAAGTGA |
| | | AATCATAGGATACAAGGCTATTGATGGTGGTGTCACTCGTGACATAGCATC |
| | | TACAGATACTTGTTTTGCTAACAAACATGCTGATTTTGACACATGGTTTAG |
| | | TCAGCGTGGTGGTAGTTATACTAATGACAAAGCTTGCCCATTGATTGCTGC |
| | | AGTCATAACAAGAGAAGTGGGTTTTGTCGTGCCTGGTTTGCCTGGCACGAT |
| | | ATTACGCACAACTAATGGTGACTTTTTGCATTTCTTACCTAGAGTTTTTAG |
| | | TGCAGTTGGTAACATCTGTTACACACCATCAAAACTTATAGAGTACACTGA |
| | | CTTTGCAACATCAGCTTGTGTTTTGGCTGCTGAATGTACAATTTTTAAAGA |
| | | TGCTTCTGGTAAGCCAGTACCATATTGTTATGATACCAATGTACTAGAAGG |
| | | TTCTGTTGCTTATGAAAGTTTACGCCCTGACACACGTTATGTGCTCATGGA |
| | | TGGCTCTATTATTCAATTTCCTAACACCTACCTTGAAGGTTCTGTTAGAGT |
| | | GGTAACAACTTTTGATTCTGAGTACTGTAGGCACGGCACTTGTGAAAGATC |
| | | AGAAGCTGGTGTTTGTGTATCTACTAGTGGTAGATGGGTACTTAACAATGA |
| | | TTATTACAGATCTTTACCAGGAGTTTTCTGTGGTGTAGATGCTGTAAATTT |
| | | ACTTACTAATATGTTTACACCACTAATTCAACCTATTGGTGCTTTGGACAT |
| | | ATCAGCATCTATAGTAGCTGGTGGTATTGTAGCTATCGTAGTAACATGCCT |
| | | TGCCTACTATTTTATGAGGTTTAGAAGAGCTTTTGGTGAATACAGTCATGT |
| | | AGTTGCCTTTAATACTTTACTATTCCTTATGTCATTCACTGTACTCTGTTT |
| | | AACACCAGTTTACTCATTCTTACCTGGTGTTTATTCTGTTATTTACTTGTA |
| | | CTTGACATTTTATCTTACTAATGATGTTTCTTTTTTAGCACATATTCAGTG |
| | | GATGGTTATGTTCACACCTTTAGTACCTTTCTGGATAACAATTGCTTATAT |
| | | CATTTGTATTTCCACAAAGCATTTCTATTGGTTCTTTAGTAATTACCTAAA |
| | | GAGACGTGTAGTCTTTAATGGTGTTTCCTTTAGTACTTTTGAAGAAGCTGC |
| | | GCTGTGCACCTTTTTGTTAAATAAAGAAATGTATCTAAAGTTGCGTAGTGA |
| | | TGTGCTATTACCTCTTACGCAATATAATAGATACTTAGCTCTTTATAATAA |
| | | GTACAAGTATTTTAGTGGAGCAATGGATACAACTAGCTACAGAGAAGCTGC |
| | | TTGTTGTCATCTCGCAAAGGCTCTCAATGACTTCAGTAACTCAGGTTCTGA |
| | | TGTTCTTTACCAACCACCACAAACCTCTATCACCTCAGCTGTTTTGCAGAG |
| | | TGGTTTTAGAAAAATGGCATTCCCATCTGGTAAAGTTGAGGGTTGTATGGT |
| | | ACAAGTAACTTGTGGTACAACTACACTTAACGGTCTTTGGCTTGATGACGT |
| | | AGTTTACTGTCCAAGACATGTGATCTGCACCTCTGAAGACATGCTTAACCC |
| | | TAATTATGAAGATTTACTCATTCGTAAGTCTAATCATAATTTCTTGGTACA |
| | | GGCTGGTAATGTTCAACTCAGGGTTATTGGACATTCTATGCAAAATTGTGT |
| | | ACTTAAGCTTAAGGTTGATACAGCCAATCCTAAGACACCTAAGTATAAGTT |
| | | TGTTCGCATTCAACCAGGACAGACTTTTTCAGTGTTAGCTTGTTACAATGG |
| | | TTCACCATCTGGTGTTTACCAATGTGCTATGAGGCCCAATTTCACTATTAA |
| | | GGGTTCATTCCTTAATGGTTCATGTGGTAGTGTTGGTTTTAACATAGATTA |
| | | TGACTGTGTCTCTTTTTGTTACATGCACCATATGGAATTACCAACTGGAGT |
| | | TCATGCTGGCACAGACTTAGAAGGTAACTTTTATGGACCTTTTGTTGACAG |
| | | GCAAACAGCACAAGCAGCTGGTACGGACACAACTATTACAGTTAATGTTTT |
| | | AGCTTGGTTGTACGCTGCTGTTATAAATGGAGACAGGTGGTTTCTCAATCG |
| | | ATTTACCACAACTCTTAATGACTTTAACCTTGTGGCTATGAAGTACAATTA |
| | | TGAACCTCTAACACAAGACCATGTTGACATACTAGGACCTCTTTCTGCTCA |
| | | AACTGGAATTGCCGTTTTAGATATGTGTGCTTCATTAAAAGAATTACTGCA |
| | | AAATGGTATGAATGGACGTACCATATTGGGTAGTGCTTTATTAGAAGATGA |
| | | ATTTACACCTTTTGATGTTGTTAGACAATGCTCAGGTGTTACTTTCCAAAG |
| | | TGCAGTGAAAAGAACAATCAAGGGTACACACCACTGGTTGTTACTCACAAT |
| | | TTTGACTTCACTTTTAGTTTTAGTCCAGAGTACTCAATGGTCTTTGTTCTT |
| | | TTTTTTGTATGAAAATGCCTTTTTACCTTTTGCTATGGGTATTATTGCTAT |
| | | GTCTGCTTTTGCAATGATGTTTGTCAAACATAAGCATGCATTTCTCTGTTT |
| | | GTTTTTGTTACCTTCTCTTGCCACTGTAGCTTATTTTAATATGGTCTATAT |
| | | GCCTGCTAGTTGGGTGATGCGTATTATGACATGGTTGGATATGGTTGATAC |
| | | TAGTTTGTCTGGTTTTAAGCTAAAAGACTGTGTTATGTATGCATCAGCTGT |
| | | AGTGTTACTAATCCTTATGACAGCAAGAACTGTGTATGATGATGGTGCTAG |
| | | GAGAGTGTGGACACTTATGAATGTCTTGACACTCGTTTATAAAGTTTATTA |
| | | TGGTAATGCTTTAGATCAAGCCATTTCCATGTGGGCTCTTATAATCTCTGT |
| | | TACTTCTAACTACTCAGGTGTAGTTACAACTGTCATGTTTTTGGCCAGAGG |
| | | TATTGTTTTTATGTGTGTTGAGTATTGCCCTATTTTCTTCATAACTGGTAA |
| | | TACACTTCAGTGTATAATGCTAGTTTATTGTTTCTTAGGCTATTTTTGTAC |
| | | TTGTTACTTTGGCCTCTTTTGTTTACTCAACCGCTACTTTAGACTGACTCT |
| | | TGGTGTTTATGATTACTTAGTTTCTACACAGGAGTTTAGATATATGAATTC |
| | | ACAGGGACTACTCCCACCCAAGAATAGCATAGATGCCTTCAAACTCAACAT |
| | | TAAATTGTTGGGTGTTGGTGGCAAACCTTGTATCAAAGTAGCCACTGTACA |
| | | GTCTAAAATGTCAGATGTAAAGTGCACATCAGTAGTCTTACTCTCAGTTTT |
| | | GCAACAACTCAGAGTAGAATCATCATCTAAATTGTGGGCTCAATGTGTCCA |
| | | GTTACACAATGACATTCTCTTAGCTAAAGATACTACTGAAGCCTTTGAAAA |
| | | AATGGTTTCACTACTTTCTGTTTTGCTTTCCATGCAGGGTGCTGTAGACAT |
| | | AAACAAGCTTTGTGAAGAAATGCTGGACAACAGGGCAACCTTACAAGCTAT |
| | | AGCCTCAGAGTTTAGTTCCCTTCCATCATATGCAGCTTTTGCTACTGCTCA |
| | | AGAAGCTTATGAGCAGGCTGTTGCTAATGGTGATTCTGAAGTTGTTCTTAA |
| | | AAAGTTGAAGAAGTCTTTGAATGTGGCTAAATCTGAATTTGACCGTGATGC |
| | | AGCCATGCAACGTAAGTTGGAAAAGATGGCTGATCAAGCTATGACCCAAAT |
| | | GTATAAACAGGCTAGATCTGAGGACAAGAGGGCAAAAGTTACTAGTGCTAT |
| | | GCAGACAATGCTTTTCACTATGCTTAGAAAGTTGGATAATGATGCACTCAA |
| | | CAACATTATCAACAATGCAAGAGATGGTTGTGTTCCCTTGAACATAATACC |
| | | TCTTACAACAGCAGCCAAACTAATGGTTGTCATACCAGACTATAACACATA |
| | | TAAAAATACGTGTGATGGTACAACATTTACTTATGCATCAGCATTGTGGGA |
| | | AATCCAACAGGTTGTAGATGCAGATAGTAAAATTGTTCAACTTAGTGAAAT |
| | | TAGTATGGACAATTCACCTAATTTAGCATGGCCTCTTATTGTAACAGCTTT |
| | | AAGGGCCAATTCTGCTGTCAAATTACAGAATAATGAGCTTAGTCCTGTTGC |
| | | ACTACGACAGATGTCTTGTGCTGCCGGTACTACACAAACTGCTTGCACTGA |
| | | TGACAATGCGTTAGCTTACTACAACACAACAAAGGGAGGTAGGTTTGTACT |
| | | TGCACTGTTATCCGATTTACAGGATTTGAAATGGGCTAGATTCCCTAAGAG |
| | | TGATGGAACTGGTACTATCTATACAGAACTGGAACCACCTTGTAGGTTTGT |
| | | TACAGACACACCTAAAGGTCCTAAAGTGAAGTATTTATACTTTATTAAAGG |
| | | ATTAAACAACCTAAATAGAGGTATGGTACTTGGTAGTTTAGCTGCCACAGT |
| | | ACGTCTACAAGCTGGTAATGCAACAGAAGTGCCTGCCAATTCAACTGTATT |
| | | ATCTTTCTGTGCTTTTGCTGTAGATGCTGCTAAAGCTTACAAAGATTATCT |
| | | AGCTAGTGGGGGACAACCAATCACTAATTGTGTTAAGATGTTGTGTACACA |
| | | CACTGGTACTGGTCAGGCAATAACAGTTACACCGGAAGCCAATATGGATCA |
| | | AGAATCCTTTGGTGGTGCATCGTGTTGTCTGTACTGCCGTTGCCACATAGA |
| | | TCATCCAAATCCTAAAGGATTTTGTGACTTAAAAGGTAAGTATGTACAAAT |
| | | ACCTACAACTTGTGCTAATGACCCTGTGGGTTTTACACTTAAAAACACAGT |
| | | CTGTACCGTCTGCGGTATGTGGAAAGGTTATGGCTGTAGTTGTGATCAACT |
| | | CCGCGAACCCATGCTTCAGTCAGCTGATGCACAATCGTTTTTAAACGGGTT |
| | | TGCGGTGTAAGTGCAGCCCGTCTTACACCGTGCGGCACAGGCACTAGTACT |
| | | GATGTCGTATACAGGGCTTTTGACATCTACAATGATAAAGTAGCTGGTTTT |
| | | GCTAAATTCCTAAAAACTAATTGTTGTCGCTTCCAAGAAAAGGACGAAGAT |
| | | GACAATTTAATTGATTCTTACTTTGTAGTTAAGAGACACACTTTCTCTAAC |
| | | TACCAACATGAAGAAACAATTTATAATTTACTTAAGGATTGTCCAGCTGTT |
| | | GCTAAACATGACTTCTTTAAGTTTAGAATAGACGGTGACATGGTACCACAT |
| | | ATATCACGTCAACGTCTTACTAAATACACAATGGCAGACCTCGTCTATGCT |
| | | TTAAGGCATTTTGATGAAGGTAATTGTGACACATTAAAAGAAATACTTGTC |
| | | ACATACAATTGTTGTGATGATGATTATTTCAATAAAAAGGACTGGTATGAT |
| | | TTTGTAGAAAACCCAGATATATTACGCGTATACGCCAACTTAGGTGAACGT |
| | | GTACGCCAAGCTTTGTTAAAAACAGTACAATTCTGTGATGCCATGCGAAAT |
| | | GCTGGTATTGTTGGTGTACTGACATTAGATAATCAAGATCTCAATGGTAAC |
| | | TGGTATGATTTCGGTGATTTCATACAAACCACGCCAGGTAGTGGAGTTCCT |
| | | GTTGTAGATTCTTATTATTCATTGTTAATGCCTATATTAACCTTGACCAGG |
| | | GCTTTAACTGCAGAGTCACATGTTGACACTGACTTAACAAAGCCTTACATT |
| | | AAGTGGGATTTGTTAAAATATGACTTCACGGAAGAGAGGTTAAAACTCTTT |
| | | GACCGTTATTTTAAATATTGGGATCAGACATACCACCCAAATTGTGTTAAC |
| | | TGTTTGGATGACAGATGCATTCTGCATTGTGCAAACTTTAATGTTTTATTC |
| | | TCTACAGTGTTCCCACCTACAAGTTTTGGACCACTAGTGAGAAAAATATTT |
| | | GTTGATGGTGTTCCATTTGTAGTTTCAACTGGATACCACTTCAGAGAGCTA |
| | | GGTGTTGTACATAATCAGGATGTAAACTTACATAGCTCTAGACTTAGTTTT |
| | | AAGGAATTACTTGTGTATGCTGCTGACCCTGCTATGCACGCTGCTTCTGGT |
| | | AATCTATTACTAGATAAACGCACTACGTGCTTTTCAGTAGCTGCACTTACT |
| | | AACAATGTTGCTTTTCAAACTGTCAAACCCGGTAATTTTAACAAAGACTTC |
| | | TATGACTTTGCTGTGTCTAAGGGTTTCTTTAAGGAAGGAAGTTCTGTTGAA |
| | | TTAAAACACTTCTTCTTTGCTCAGGATGGTAATGCTGCTATCAGCGATTAT |
| | | GACTACTATCGTTATAATCTACCAACAATGTGTGATATCAGACAACTACTA |
| | | TTTGTAGTTGAAGTTGTTGATAAGTACTTTGATTGTTACGATGGTGGCTGT |
| | | ATTAATGCTAACCAAGTCATCGTCAACAACCTAGACAAATCAGCTGGTTTT |
| | | CCATTTAATAAATGGGGTAAGGCTAGACTTTATTATGATTCAATGAGTTAT |
| | | GAGGATCAAGATGCACTTTTCGCATATACAAAACGTAATGTCATCCCTACT |
| | | ATAACTCAAATGAATCTTAAGTATGCCATTAGTGCAAAGAATAGAGCTCGC |
| | | ACCGTAGCTGGTGTCTCTATCTGTAGTACTATGACCAATAGACAGTTTCAT |
| | | CAAAAATTATTGAAATCAATAGCCGCCACTAGAGGAGCTACTGTAGTAATT |
| | | GGAACAAGCAAATTCTATGGTGGTTGGCACAACATGTTAAAAACTGTTTAT |
| | | AGTGATGTAGAAAACCCTCACCTTATGGGTTGGGATTATCCTAAATGTGAT |
| | | AGAGCCATGCCTAACATGCTTAGAATTATGGCCTCACTTGTTCTTGCTCGC |
| | | AAACATACAACGTGTTGTAGCTTGTCACACCGTTTCTATAGATTAGCTAAT |
| | | GAGTGTGCTCAAGTATTGAGTGAAATGGTCATGTGTGGCGGTTCACTATAT |
| | | GTTAAACCAGGTGGAACCTCATCAGGAGATGCCACAACTGCTTATGCTAAT |
| | | AGTGTTTTTAACATTTGTCAAGCTGTCACGGCCAATGTTAATGCACTTTTA |
| | | TCTACTGATGGTAACAAAATTGCCGATAAGTATGTCCGCAATTTACAACAC |
| | | AGACTTTATGAGTGTCTCTATAGAAATAGAGATGTTGACACAGACTTTGTG |
| | | AATGAGTTTTACGCATATTTGCGTAAACATTTCTCAATGATGATACTCTCT |
| | | GACGATGCTGTTGTGTGTTTCAATAGCACTTATGCATCTCAAGGTCTAGTG |
| | | GCTAGCATAAAGAACTTTAAGTCAGTTCTTTATTATCAAAACAATGTTTTT |
| | | ATGTCTGAAGCAAAATGTTGGACTGAGACTGACCTTACTAAAGGACCTCAT |
| | | GAATTTTGCTCTCAACATACAATGCTAGTTAAACAGGGTGATGATTATGTG |
| | | TACCTTCCTTACCCAGATCCATCAAGAATCCTAGGGGCCGGCTGTTTTGTA |
| | | GATGATATCGTAAAAACAGATGGTACACTTATGATTGAACGGTTCGTGTCT |
| | | TTAGCTATAGATGCTTACCCACTTACTAAACATCCTAATCAGGAGTATGCT |
| | | GATGTCTTTCATTTGTACTTACAATACATAAGAAAGCTACATGATGAGTTA |
| | | ACAGGACACATGTTAGACATGTATTCTGTTATGCTTACTAATGATAACACT |
| | | TCAAGGTATTGGGAACCTGAGTTTTATGAGGCTATGTACACACCGCATACA |
| | | GTCTTACAGGCTGTTGGGGCTTGTGTTCTTTGCAATTCACAGACTTCATTA |
| | | AGATGTGGTGCTTGCATACGTAGACCATTCTTATGTTGTAAATGCTGTTAC |
| | | GACCATGTCATATCAACATCACATAAATTAGTCTTGTCTGTTAATCCGTAT |
| | | GTTTGCAATGCTCCAGGTTGTGATGTCACAGATGTGACTCAACTTTACTTA |
| | | GGAGGTATGAGCTATTATTGTAAATCACATAAACCACCCATTAGTTTTCCA |
| | | TTGTGTGCTAATGGACAAGTTTTTGGTTTATATAAAAATACATGTGTTGGT |
| | | AGCGATAATGTTACTGACTTTAATGCAATTGCAACATGTGACTGGACAAAT |
| | | GCTGGTGATTACATTTTAGCTAACACCTGTACTGAAAGACTCAAGCTTTTT |
| | | GCAGCAGAAACGCTCAAAGCTACTGAGGAGACATTTAAACTGTCTTATGGT |
| | | ATTGCTACTGTACGTGAAGTGCTGTCTGACAGAGAATTACATCTTTCATGG |
| | | GAAGTTGGTAAACCTAGACCACCACTTAACCGAAATTATGTCTTTACTGGT |
| | | TATCGTGTAACTAAAAACAGTAAAGTACAAATAGGAGAGTACACCTTTGAA |
| | | AAAGGTGACTATGGTGATGCTGTTGTTTACCGAGGTACAACAACTTACAAA |
| | | TTAAATGTTGGTGATTATTTTGTGCTGACATCACATACAGTAATGCCATTA |
| | | AGTGCACCTACACTAGTGCCACAAGAGCACTATGTTAGAATTACTGGCTTA |
| | | TACCCAACACTCAATATCTCAGATGAGTTTTCTAGCAATGTTGCAAATTAT |
| | | CAAAAGGTTGGTATGCAAAAGTATTCTACACTCCAGGGACCACCTGGTACT |
| | | GGTAAGAGTCATTTTGCTATTGGCCTAGCTCTCTACTACCCTTCTGCTCGC |
| | | ATAGTGTATACAGCTTGCTCTCATGCCGCTGTTGATGCACTATGTGAGAAG |
| | | GCATTAAAATATTTGCCTATAGATAAATGTAGTAGAATTATACCTGCACGT |
| | | GCTCGTGTAGAGTGTTTTGATAAATTCAAAGTGAATTCAACATTAGAACAG |
| | | TATGTCTTTTGTACTGTAAATGCATTGCCTGAGACGACAGCAGATATAGTT |
| | | GTCTTTGATGAAATTTCAATGGCCACAAATTATGATTTGAGTGTTGTCAAT |
| | | GCCAGATTACGTGCTAAGCACTATGTGTACATTGGCGACCCTGCTCAATTA |
| | | CCTGCACCACGCACATTGCTAACTAAGGGCACACTAGAACCAGAATATTTC |
| | | AATTCAGTGTGTAGACTTATGAAAACTATAGGTCCAGACATGTTCCTCGGA |
| | | ACTTGTCGGCGTTGTCCTGCTGAAATTGTTGACACTGTGAGTGCTTTGGTT |
| | | TATGATAATAAGCTTAAAGCACATAAAGACAAATCAGCTCAATGCTTTAAA |
| | | ATGTTTTATAAGGGTGTTATCACGCATGATGTTTCATCTGCAATTAACAGG |
| | | CCACAAATAGGCGTGGTAAGAGAATTCCTTACACGTAACCTTGCTTGGAGA |
| | | AAAGCTGTCTTTATTTCACCTTATAATTCACAGAATGCTGTAGCCTCAAAG |
| | | ATTTTGGGACTACCAACTCAAACTGTTGATTCATCACAGGGCTCAGAATGT |
| | | GACTATGTCATATTCACTCAAACCACTGAAACAGCTCACTCTTGTAATGTA |
| | | AACAGATTTAATGTTGCTATTACCAGAGCAAAAGTAGGCATACTTTGCATA |
| | | ATGTCTGATAGAGACCTTTATGACAAGTTGCAATTTACAAGTCTTGAAATT |
| | | CCACGTAGGAATGTGGCAACTTTACAAGCTGAAAATGTAACAGGACTTTTT |
| | | AAAGATTGTAGTAAGGTAATCACTGGGTTACATCCTACACAGGCACCTACA |
| | | CACCTCAGTGTTGACACTAAATTCAAAACTGAAGGTTTATGTGTTGACATA |
| | | CCTGGCATACCTAAGGACATGACCTATAGAAGACTCATCTCTATGATGGGT |
| | | TTTAAAATGAATTATCAAGTTAATGGTTACCCTAACATGTTTATCACCCGC |
| | | GAAGAAGCTATAAGACATGTACGTGCATGGATTGGCTTCGATGTCGAGGGG |
| | | TGTCATGCTACTAGAGAAGCTGTTGGTACCAATTTACCTTTACAGCTAGGT |
| | | TTTTCTACAGGTGTTAACCTAGTTGCTGTACCTACAGGTTATGTTGATACA |
| | | CCTAATAATACAGATTTTTCCAGAGTTAGTGCTAAACCACCGCCTGGAGAT |
| | | CAATTTAAACACCTCATACCACTTATGTACAAAGGACTTCCTTGGAATGTA |
| | | GTGCGTATAAAGATTGTACAAATGTTAAGTGACACACTTAAAAATCTCTCT |
| | | GACAGAGTCGTATTTGTCTTATGGGCACATGGCTTTGAGTTGACATCTATG |
| | | AAGTATTTTGTGAAAATAGGACCTGAGCGCACCTGTTGTCTATGTGATAGA |
| | | CGTGCCACATGCTTTTCCACTGCTTCAGACACTTATGCCTGTTGGCATCAT |
| | | TCTATTGGATTTGATTACGTCTATAATCCGTTTATGATTGATGTTCAACAA |
| | | TGGGGTTTTACAGGTAACCTACAAAGCAACCATGATCTGTATTGTCAAGTC |
| | | CATGGTAATGCACATGTAGCTAGTTGTGATGCAATCATGACTAGGTGTCTA |
| | | GCTGTCCACGAGTGCTTTGTTAAGCGTGTTGACTGGACTATTGAATATCCT |
| | | ATAATTGGTGATGAACTGAAGATTAATGCGGCTTGTAGAAAGGTTCAACAC |
| | | ATGGTTGTTAAAGCTGCATTATTAGCAGACAAATTCCCAGTTCTTCACGAC |
| | | ATTGGTAACCCTAAAGCTATTAAGTGTGTACCTCAAGCTGATGTAGAATGG |
| | | AAGTTCTATGATGCACAGCCTTGTAGTGACAAAGCTTATAAAATAGAAGAA |
| | | TTATTCTATTCTTATGCCACACATTCTGACAAATTCACAGATGGTGTATGC |
| | | CTATTTTGGAATTGCAATGTCGATAGATATCCTGCTAATTCCATTGTTTGT |
| | | AGATTTGACACTAGAGTGCTATCTAACCTTAACTTGCCTGGTTGTGATGGT |
| | | GGCAGTTTGTATGTAAATAAACATGCATTCCACACACCAGCTTTTGATAAA |
| | | AGTGCTTTTGTTAATTTAAAACAATTACCATTTTTCTATTACTCTGACAGT |
| | | CCATGTGAGTCTCATGGAAAACAAGTAGTGTCAGATATAGATTATGTACCA |
| | | CTAAAGTCTGCTACGTGTATAACACGTTGCAATTTAGGTGGTGCTGTCTGT |
| | | AGACATCATGCTAATGAGTACAGATTGTATCTCGATGCTTATAACATGATG |
| | | ATCTCAGCTGGCTTTAGCTTGTGGGTTTACAAACAATTTGATACTTATAAC |
| | | CTCTGGAACACTTTTACAAGACTTCAGAGTTTAGAAAATGTGGCTTTTAAT |
| | | GTTGTAAATAAGGGACACTTTGATGGACAACAGGGTGAAGTACCAGTTTCT |
| | | ATCATTAATAACACTGTTTACACAAAAGTTGATGGTGTTGATGTAGAATTG |
| | | TTTGAAAATAAAACAACATTACCTGTTAATGTAGCATTTGAGCTTTGGGCT |
| | | AAGCGCAACATTAAACCAGTACCAGAGGTGAAAATACTCAATAATTTGGGT |
| | | GTGGACATTGCTGCTAATACTGTGATCTGGGACTACAAAAGAGATGCTCCA |
| | | GCACATATATCTACTATTGGTGTTTGTTCTATGACTGACATAGCCAAGAAA |
| | | CCAACTGAAACGATTTGTGCACCACTCACTGTCTTTTTTGATGGTAGAGTT |
| | | GATGGTCAAGTAGACTTATTTAGAAATGCCCGTAATGGTGTTCTTATTACA |
| | | GAAGGTAGTGTTAAAGGTTTACAACCATCTGTAGGTCCCAAACAAGCTAGT |
| | | CTTAATGGAGTCACATTAATTGGAGAAGCCGTAAAAACACAGTTCAATTAT |
| | | TATAAGAAAGTTGATGGTGTTGTCCAACAATTACCTGAAACTTACTTTACT |
| | | CAGAGTAGAAATTTACAAGAATTTAAACCCAGGAGTCAAATGGAAATTGAT |
| | | TTCTTAGAATTAGCTATGGATGAATTCATTGAACGGTATAAATTAGAAGGC |
| | | TATGCCTTCGAACATATCGTTTATGGAGATTTTAGTCATAGTCAGTTAGGT |
| | | GGTTTACATCTACTGATTGGACTAGCTAAACGTTTTAAGGAATCACCTTTT |
| | | GAATTAGAAGATTTTATTCCTATGGACAGTACAGTTAAAAACTATTTCATA |
| | | ACAGATGCGCAAACAGGTTCATCTAAGTGTGTGTGTTCTGTTATTGATTTA |
| | | TTACTTGATGATTTTGTTGAAATAATAAAATCCCAAGATTTATCTGTAGTT |
| | | TCTAAGGTTGTCAAAGTGACTATTGACTATACAGAAATTTCATTTATGCTT |
| | | TGGTGTAAAGATGGCCATGTAGAAACATTTTACCCAAAATTACAATCTAGT |
| | | CAAGCGTGGCAACCGGGTGTTGCTATGCCTAATCTTTACAAAATGCAAAGA |
| | | ATGCTATTAGAAAAGTGTGACCTTCAAAATTATGGTGATAGTGCAACATTA |
| | | CCTAAAGGCATAATGATGAATGTCGCAAAATATACTCAACTGTGTCAATAT |
| | | TTAAACACATTAACATTAGCTGTACCCTATAATATGAGAGTTATACATTTT |
| | | GGTGCTGGTTCTGATAAAGGAGTTGCACCAGGTACAGCTGTTTTAAGACAG |
| | | TGGTTGCCTACGGGTACGCTGCTTGTCGATTCAGATCTTAATGACTTTGTC |
| | | TCTGATGCAGATTCAACTTTGATTGGTGATTGTGCAACTGTACATACAGCT |
| | | AATAAATGGGATCTCATTATTAGTGATATGTACGACCCTAAGACTAAAAAT |
| | | GTTACAAAAGAAAATGACTCTAAAGAGGGTTTTTTCACTTACATTTGTGGG |
| | | TTTATACAACAAAAGCTAGCTCTTGGAGGTTCCGTGGCTATAAAGATAACA |
| | | GAACATTCTTGGAATGCTGATCTTTATAAGCTCATGGGACACTTCGCATGG |
| | | TGGACAGCCTTTGTTACTAATGTGAATGCGTCATCATCTGAAGCATTTTTA |
| | | ATTGGATGTAATTATCTTGGCAAACCACGCGAACAAATAGATGGTTATGTC |
| | | ATGCATGCAAATTACATATTTTGGAGGAATACAAATCCAATTCAGTTGTCT |
| | | TCCTATTCTTTATTTGACATGAGTAAATTTCCCCTTAAATTAAGGGGTACT |
| | | GCTGTTATGTCTTTAAAAGAAGGTCAAATCAATGATATGATTTTATCTCTT |
| | | CTTAGTAAAGGTAGACTTATAATTAGAGAAAACAACAGAGTTGTTATTTCT |
| | | AGTGATGTTCTTGTTAACAACTAAACGAACAATGTTTGTTTTTCTTGTTTT |
| | | ATTGCCACTAGTCTCTAGTCAGTGTGTTAATCTTACAACCAGAACTCAATT |
| | | ACCCCCTGCATACACTAATTCTTTCACACGTGGTGTTTATTACCCTGACAA |
| | | AGTTTTCAGATCCTCAGTTTTACATTCAACTCAGGACTTGTTCTTACCTTT |
| | | CTTTTCCAATGTTACTTGGTTCCATGCTATACATGTCTCTGGGACCAATGG |
| | | TACTAAGAGGTTTGATAACCCTGTCCTACCATTTAATGATGGTGTTTATTT |
| | | TGCTTCCACTGAGAAGTCTAACATAATAAGAGGCTGGATTTTTGGTACTAC |
| | | TTTAGATTCGAAGACCCAGTCCCTACTTATTGTTAATAACGCTACTAATGT |
| | | TGTTATTAAAGTCTGTGAATTTCAATTTTGTAATGATCCATTTTTGGGTGT |
| | | TTATTACCACAAAAACAACAAAAGTTGGATGGAAAGTGAGTTCAGAGTTTA |
| | | TTCTAGTGCGAATAATTGCACTTTTGAATATGTCTCTCAGCCTTTTCTTAT |
| | | GGACCTTGAAGGAAAACAGGGTAATTTCAAAAATCTTAGGGAATTTGTGTT |
| | | TAAGAATATTGATGGTTATTTTAAAATATATTCTAAGCACACGCCTATTAA |
| | | TTTAGTGCGTGATCTCCCTCAGGGTTTTTCGGCTTTAGAACCATTGGTAGA |
| | | TTTGCCAATAGGTATTAACATCACTAGGTTTCAAACTTTACTTGCTTTACA |
| | | TAGAAGTTATTTGACTCCTGGTGATTCTTCTTCAGGTTGGACAGCTGGTGC |
| | | TGCAGCTTATTATGTGGGTTATCTTCAACCTAGGACTTTTCTATTAAAATA |
| | | TAATGAAAATGGAACCATTACAGATGCTGTAGACTGTGCACTTGACCCTCT |
| | | CTCAGAAACAAAGTGTACGTTGAAATCCTTCACTGTAGAAAAAGGAATCTA |
| | | TCAAACTTCTAACTTTAGAGTCCAACCAACAGAATCTATTGTTAGATTTCC |
| | | TAATATTACAAACTTGTGCCCTTTTGGTGAAGTTTTTAACGCCACCAGATT |
| | | TGCATCTGTTTATGCTTGGAACAGGAAGAGAATCAGCAACTGTGTTGCTGA |
| | | TTATTCTGTCCTATATAATTCCGCATCATTTTCCACTTTTAAGTGTTATGG |
| | | AGTGTCTCCTACTAAATTAAATGATCTCTGCTTTACTAATGTCTATGCAGA |
| | | TTCATTTGTAATTAGAGGTGATGAAGTCAGACAAATCGCTCCAGGGCAAAC |
| | | TGGAAAGATTGCTGATTATAATTATAAATTACCAGATGATTTTACAGGCTG |
| | | CGTTATAGCTTGGAATTCTAACAATCTTGATTCTAAGGTTGGTGGTAATTA |
| | | TAATTACCTGTATAGATTGTTTAGGAAGTCTAATCTCAAACCTTTTGAGAG |
| | | AGATATTTCAACTGAAATCTATCAGGCCGGTAGCACACCTTGTAATGGTGT |
| | | TGAAGGTTTTAATTGTTACTTTCCTTTACAATCATATGGTTTCCAACCCAC |
| | | TAATGGTGTTGGTTACCAACCATACAGAGTAGTAGTACTTTCTTTTGAACT |
| | | TCTACATGCACCAGCAACTGTTTGTGGACCTAAAAAGTCTACTAATTTGGT |
| | | TAAAAACAAATGTGTCAATTTCAACTTCAATGGTTTAACAGGCACAGGTGT |
| | | TCTTACTGAGTCTAACAAAAAGTTTCTGCCTTTCCAACAATTTGGCAGAGA |
| | | CATTGCTGACACTACTGATGCTGTCCGTGATCCACAGACACTTGAGATTCT |
| | | TGACATTACACCATGTTCTTTTGGTGGTGTCAGTGTTATAACACCAGGAAC |
| | | AAATACTTCTAACCAGGTTGCTGTTCTTTATCAGGATGTTAACTGCACAGA |
| | | AGTCCCTGTTGCTATTCATGCAGATCAACTTACTCCTACTTGGCGTGTTTA |
| | | TTCTACAGGTTCTAATGTTTTTCAAACACGTGCAGGCTGTTTAATAGGGGC |
| | | TGAACATGTCAACAACTCATATGAGTGTGACATACCCATTGGTGCAGGTAT |
| | | ATGCGCTAGTTATCAGACTCAGACTAATTCTCCTCGGCGGGCACGTAGTGT |
| | | AGCTAGTCAATCCATCATTGCCTACACTATGTCACTTGGTGCAGAAAATTC |
| | | AGTTGCTTACTCTAATAACTCTATTGCCATACCCACAAATTTTACTATTAG |
| | | TGTTACCACAGAAATTCTACCAGTGTCTATGACCAAGACATCAGTAGATTG |
| | | TACAATGTACATTTGTGGTGATTCAACTGAATGCAGCAATCTTTTGTTGCA |
| | | ATATGGCAGTTTTTGTACACAATTAAACCGTGCTTTAACTGGAATAGCTGT |
| | | TGAACAAGACAAAAACACCCAAGAAGTTTTTGCACAAGTCAAACAAATTTA |
| | | CAAAACACCACCAATTAAAGATTTTGGTGGTTTTAATTTTTCACAAATATT |
| | | ACCAGATCCATCAAAACCAAGCAAGAGGTCATTTATTGAAGATCTACTTTT |
| | | CAACAAAGTGACACTTGCAGATGCTGGCTTCATCAAACAATATGGTGATTG |
| | | CCTTGGTGATATTGCTGCTAGAGACCTCATTTGTGCACAAAAGTTTAACGG |
| | | CCTTACTGTTTTGCCACCTTTGCTCACAGATGAAATGATTGCTCAATACAC |
| | | TTCTGCACTGTTAGCGGGTACAATCACTTCTGGTTGGACCTTTGGTGCAGG |
| | | TGCTGCATTACAAATACCATTTGCTATGCAAATGGCTTATAGGTTTAATGG |
| | | TATTGGAGTTACACAGAATGTTCTCTATGAGAACCAAAAATTGATTGCCAA |
| | | CCAATTTAATAGTGCTATTGGCAAAATTCAAGACTCACTTTCTTCCACAGC |
| | | AAGTGCACTTGGAAAACTTCAAGATGTGGTCAACCAAAATGCACAAGCTTT |
| | | AAACACGCTTGTTAAACAACTTAGCTCCAATTTTGGTGCAATTTCAAGTGT |
| | | TTTAAATGATATCCTTTCACGTCTTGACAAAGTTGAGGCTGAAGTGCAAAT |
| | | TGATAGGTTGATCACAGGCAGACTTCAAAGTTTGCAGACATATGTGACTCA |
| | | ACAATTAATTAGAGCTGCAGAAATCAGAGCTTCTGCTAATCTTGCTGCTAC |
| | | TAAAATGTCAGAGTGTGTACTTGGACAATCAAAAAGAGTTGATTTTTGTGG |
| | | AAAGGGCTATCATCTTATGTCCTTCCCTCAGTCAGCACCTCATGGTGTAGT |
| | | CTTCTTGCATGTGACTTATGTCCCTGCACAAGAAAAGAACTTCACAACTGC |
| | | TCCTGCCATTTGTCATGATGGAAAAGCACACTTTCCTCGTGAAGGTGTCTT |
| | | TGTTTCAAATGGCACACACTGGTTTGTAACACAAAGGAATTTTTATGAACC |
| | | ACAAATCATTACTACAGACAACACATTTGTGTCTGGTAACTGTGATGTTGT |
| | | AATAGGAATTGTCAACAACACAGTTTATGATCCTTTGCAACCTGAATTAGA |
| | | CTCATTCAAGGAGGAGTTAGATAAATATTTTAAGAATCATACATCACCAGA |
| | | TGTTGATTTAGGTGACATCTCTGGCATTAATGCTTCAGTTGTAAACATTCA |
| | | AAAAGAAATTGACCGCCTCAATGAGGTTGCCAAGAATTTAAATGAATCTCT |
| | | CATCGATCTCCAAGAACTTGGAAAGTATGAGCAGTATATAAAATGGCCATG |
| | | GTACATTTGGCTAGGTTTTATAGCTGGCTTGATTGCCATAGTAATGGTGAC |
| | | AATTATGCTTTGCTGTATGACCAGTTGCTGTAGTTGTCTCAAGGGCTGTTG |
| | | TTCTTGTGGATCCTGCTGCAAATTTGATGAAGACGACTCTGAGCCAGTGCT |
| | | CAAAGGAGTCAAATTACATTACACATAAACGAACTTATGGATTTGTTTATG |
| | | AGAATCTTCACAATTGGAACTGTAACTTTGAAGCAAGGTGAAATCAAGGAT |
| | | GCTACTCCTTCAGATTTTGTTCGCGCTACTGCAACGATACCGATACAAGCC |
| | | TCACTCCCTTTCGGATGGCTTATTGTTGGCGTTGCACTTCTTGCTGTTTTT |
| | | CAGAGCGCTTCCAAAATCATAACCCTCAAAAAGAGATGGCAACTAGCACTC |
| | | TCCAAGGGTGTTCACTTTGTTTGCAACTTGCTGTTGTTGTTTGTAACAGTT |
| | | TACTCACACCTTTTGCTCGTTGCTGCTGGCCTTGAAGCCCCTTTTCTCTAT |
| | | CTTTATGCTTTAGTCTACTTCTTGCAGAGTATAAACTTTGTAAGAATAATA |
| | | ATGAGGCTTTGGCTTTGCTGGAAATGCCGTTCCAAAAACCCATTACTTTAT |
| | | GATGCCAACTATTTTCTTTGCTGGCATACTAATTGTTACGACTATTGTATA |
| | | CCTTACAATAGTGTAACTTCTTCAATTGTCATTACTTCAGGTGATGGCACA |
| | | ACAAGTCCTATTTCTGAACATGACTACCAGATTGGTGGTTATACTGAAAAA |
| | | TGGGAATCTGGAGTAAAAGACTGTGTTGTATTACACAGTTACTTCACTTCA |
| | | GACTATTACCAGCTGTACTCAACTCAATTGAGTACAGACACTGGTGTTGAA |
| | | CATGTTACCTTCTTCATCTACAATAAAATTGTTGATGAGCCTGAAGAACAT |
| | | GTCCAAATTCACACAATCGACGGTTCATCCGGAGTTGTTAATCCAGTAATG |
| | | GAACCAATTTATGATGAACCGACGACGACTACTAGCGTGCCTTTGTAAGCA |
| | | CAAGCTGATGAGTACGAACTTATGTACTCATTCGTTTCGGAAGAGACAGGT |
| | | ACGTTAATAGTTAATAGCGTACTTCTTTTTCTTGCTTTCGTGGTATTCTTG |
| | | CTAGTTACACTAGCCATCCTTACTGCGCTTCGATTGTGTGCGTACTGCTGC |
| | | AATATTGTTAACGTGAGTCTTGTAAAACCTTCTTTTTACGTTTACTCTCGT |
| | | GTTAAAAATCTGAATTCTTCTAGAGTTCCTGATCTTCTGGTCTAAACGAAC |
| | | TAAATATTATATTAGTTTTTCTGTTTGGAACTTTAATTTTAGCCATGGCAG |
| | | ATTCCAACGGTACTATTACCGTTGAAGAGCTTAAAAAGCTCCTTGAACAAT |
| | | GGAACCTAGTAATAGGTTTCCTATTCCTTACATGGATTTGTCTTCTACAAT |
| | | TTGCCTATGCCAACAGGAATAGGTTTTTGTATATAATTAAGTTAATTTTCC |
| | | TCTGGCTGTTATGGCCAGTAACTTTAGCTTGTTTTGTGCTTGCTGCTGTTT |
| | | ACAGAATAAATTGGATCACCGGTGGAATTGCTATCGCAATGGCTTGTCTTG |
| | | TAGGCTTGATGTGGCTCAGCTACTTCATTGCTTCTTTCAGACTGTTTGCGC |
| | | GTACGCGTTCCATGTGGTCATTCAATCCAGAAACTAACATTCTTCTCAACG |
| | | TGCCACTCCATGGCACTATTCTGACCAGACCGCTTCTAGAAAGTGAACTCG |
| | | TAATCGGAGCTGTGATCCTTCGTGGACATCTTCGTATTGCTGGACACCATC |
| | | TAGGACGCTGTGACATCAAGGACCTGCCTAAAGAAATCACTGTTGCTACAT |
| | | CACGAACGCTTTCTTATTACAAATTGGGAGCTTCGCAGCGTGTAGCAGGTG |
| | | ACTCAGGTTTTGCTGCATACAGTCGCTACAGGATTGGCAACTATAAATTAA |
| | | ACACAGACCATTCCAGTAGCAGTGACAATATTGCTTTGCTTGTACAGTAAG |
| | | TGACAACAGATGTTTCATCTCGTTGACTTTCAGGTTACTATAGCAGAGATA |
| | | TTACTAATTATTATGAGGACTTTTAAAGTTTCCATTTGGAATCTTGATTAC |
| | | ATCATAAACCTCATAATTAAAAATTTATCTAAGTCACTAACTGAGAATAAA |
| | | TATTCTCAATTAGATGAAGAGCAACCAATGGAGATTGATTAAACGAACATG |
| | | AAAATTATTCTTTTCTTGGCACTGATAACACTCGCTACTTGTGAGCTTTAT |
| | | CACTACCAAGAGTGTGTTAGAGGTACAACAGTACTTTTAAAAGAACCTTGC |
| | | TCTTCTGGAACATACGAGGGCAATTCACCATTTCATCCTCTAGCTGATAAC |
| | | AAATTTGCACTGACTTGCTTTAGCACTCAATTTGCTTTTGCTTGTCCTGAC |
| | | GGCGTAAAACACGTCTATCAGTTACGTGCCAGATCAGTTTCACCTAAACTG |
| | | TTCATCAGACAAGAGGAAGTTCAAGAACTTTACTCTCCAATTTTTCTTATT |
| | | GTTGCGGCAATAGTGTTTATAACACTTTGCTTCACACTCAAAAGAAAGACA |
| | | GAATGATTGAACTTTCATTAATTGACTTCTATTTGTGCTTTTTAGCCTTTC |
| | | TGCTATTCCTTGTTTTAATTATGCTTATTATCTTTTGGTTCTCACTTGAAC |
| | | TGCAAGATCATAATGAAACTTGTCACGCCTAAACGAACATGAAATTTCTTG |
| | | TTTTCTTAGGAATCATCACAACTGTAGCTGCATTTCACCAAGAATGTAGTT |
| | | TACAGTCATGTACTCAACATCAACCATATGTAGTTGATGACCCGTGTCCTA |
| | | TTCACTTCTATTCTAAATGGTATATTAGAGTAGGAGCTAGAAAATCAGCAC |
| | | CTTTAATTGAATTGTGCGTGGATGAGGCTGGTTCTAAATCACCCATTCAGT |
| | | ACATCGATATCGGTAATTATACAGTTTCCTGTTCACCTTTTACAATTAATT |
| | | GCCAGGAACCTAAATTGGGTAGTCTTGTAGTGCGTTGTTCGTTCTATGAAG |
| | | ACTTTTTAGAGTATCATGACGTTCGTGTTGTTTTAGATTTCATCTAAACGA |
| | | ACAAACTAAAATGTCTGATAATGGACCCCAAAATCAGCGAAATGCACCCCG |
| | | CATTACGTTTGGTGGACCCTCAGATTCAACTGGCAGTAACCAGAATGGAGA |
| | | ACGCAGTGGGGCGCGATCAAAACAACGTCGGCCCCAAGGTTTACCCAATAA |
| | | TACTGCGTCTTGGTTCACCGCTCTCACTCAACATGGCAAGGAAGACCTTAA |
| | | ATTCCCTCGAGGACAAGGCGTTCCAATTAACACCAATAGCAGTCCAGATGA |
| | | CCAAATTGGCTACTACCGAAGAGCTACCAGACGAATTCGTGGTGGTGACGG |
| | | TAAAATGAAAGATCTCAGTCCAAGATGGTATTTCTACTACCTAGGAACTGG |
| | | GCCAGAAGCTGGACTTCCCTATGGTGCTAACAAAGACGGCATCATATGGGT |
| | | TGCAACTGAGGGAGCCTTGAATACACCAAAAGATCACATTGGCACCCGCAA |
| | | TCCTGCTAACAATGCTGCAATCGTGCTACAACTTCCTCAAGGAACAACATT |
| | | GCCAAAAGGCTTCTACGCAGAAGGGAGCAGAGGCGGCAGTCAAGCCTCTTC |
| | | TCGTTCCTCATCACGTAGTCGCAACAGTTCAAGAAATTCAACTCCAGGCAG |
| | | CAGTAGGGGAACTTCTCCTGCTAGAATGGCTGGCAATGGCGGTGATGCTGC |
| | | TCTTGCTTTGCTGCTGCTTGACAGATTGAACCAGCTTGAGAGCAAAATGTC |
| | | TGGTAAAGGCCAACAACAACAAGGCCAAACTGTCACTAAGAAATCTGCTGC |
| | | TGAGGCTTCTAAGAAGCCTCGGCAAAAACGTACTGCCACTAAAGCATACAA |
| | | TGTAACACAAGCTTTCGGCAGACGTGGTCCAGAACAAACCCAAGGAAATTT |
| | | TGGGGACCAGGAACTAATCAGACAAGGAACTGATTACAAACATTGGCCGCA |
| | | AATTGCACAATTTGCCCCCAGCGCTTCAGCGTTCTTCGGAATGTCGCGCAT |
| | | TGGCATGGAAGTCACACCTTCGGGAACGTGGTTGACCTACACAGGTGCCAT |
| | | CAAATTGGATGACAAAGATCCAAATTTCAAAGATCAAGTCATTTTGCTGAA |
| | | TAAGCATATTGACGCATACAAAACATTCCCACCAACAGAGCCTAAAAAGGA |
| | | CAAAAAGAAGAAGGCTGATGAAACTCAAGCCTTACCGCAGAGACAGAAGAA |
| | | ACAGCAAACTGTGACTCTTCTTCCTGCTGCAGATTTGGATGATTTCTCCAA |
| | | ACAATTGCAACAATCCATGAGCAGTGCTGACTCAACTCAGGCCTAAACTCA |
| | | TGCAGACCACACAAGGCAGATGGGCTATATAAACGTTTTCGCTTTTCCGTT |
| | | TACGATATATAGTCTACTCTTGTGCAGAATGAATTCTCGTAACTACATAGC |
| | | ACAAGTAGATGTAGTTAACTTTAATCTCACATA |
| 4 | CoV-2 S-Protein | MFVFLVLLPLVSSQCVNLTTRTQLPPAYTNSFTRGVYYPDKVFRSSVLHST |
| | Amino Acid | QDLFLPFFSNVTWFHAIHVSGTNGTKRFDNPVLPFNDGVYFASTEKSNIIR |
| | (Sequence from | GWIFGTTLDSKTQSLLIVNNATNVVIKVCEFQFCNDPFLGVYYHKNNKSWM |
| | vector, identical | ESEFRVYSSANNCTFEYVSQPFLMDLEGKQGNFKNLREFVFKNIDGYFKIY |
| | to SEQ ID NO: 2) | SKHTPINLVRDLPQGFSALEPLVDLPIGINITRFQTLLALHRSYLTPGDSS |
| | | SGWTAGAAAYYVGYLQPRTFLLKYNENGTITDAVDCALDPLSETKCTLKSF |
| | | TVEKGIYQTSNFRVQPTESIVRFPNITNLCPFGEVFNATRFASVYAWNRKR |
| | | ISNCVADYSVLYNSASFSTFKCYGVSPTKLNDLCFTNVYADSFVIRGDEVR |
| | | QIAPGQTGKIADYNYKLPDDFTGCVIAWNSNNLDSKVGGNYNYLYRLFRKS |
| | | NLKPFERDISTEIYQAGSTPCNGVEGFNCYFPLQSYGFQPTNGVGYQPYRV |
| | | VVLSFELLHAPATVCGPKKSTNLVKNKCVNFNFNGLTGTGVLTESNKKFLP |
| | | FQQFGRDIADTTDAVRDPQTLEILDITPCSFGGVSVITPGTNTSNQVAVLY |
| | | QDVNCTEVPVAIHADQLTPTWRVYSTGSNVFQTRAGCLIGAEHVNNSYECD |
| | | IPIGAGICASYQTQTNSPRRARSVASQSIIAYTMSLGAENSVAYSNNSIAI |
| | | PTNFTISVTTEILPVSMTKTSVDCTMYICGDSTECSNLLLQYGSFCTQLNR |
| | | ALTGIAVEQDKNTQEVFAQVKQIYKTPPIKDFGGFNFSQILPDPSKPSKRS |
| | | FIEDLLFNKVTLADAGFIKQYGDCLGDIAARDLICAQKFNGLTVLPPLLTD |
| | | EMIAQYTSALLAGTITSGWTFGAGAALQIPFAMQMAYRFNGIGVTQNVLYE |
| | | NQKLIANQFNSAIGKIQDSLSSTASALGKLQDVVNQNAQALNTLVKQLSSN |
| | | FGAISSVLNDILSRLDKVEAEVQIDRLITGRLQSLQTYVTQQLIRAAEIRA |
| | | SANLAATKMSECVLGQSKRVDFCGKGYHLMSFPQSAPHGVVFLHVTYVPAQ |
| | | EKNFTTAPAICHDGKAHFPREGVFVSNGTHWFVTQRNEYEPQIITTDNTEV |
| | | SGNCDVVIGIVNNTVYDPLQPELDSFKEELDKYEKNHTSPDVDLGDISGIN |
| | | ASVVNIQKEIDRLNEVAKNLNESLIDLQELGKYEQYIKWPWYIWLGFIAGL |
| | | IAIVMVTIMLCCMTSCCSCLKGCCSCGSCCKFDEDDSEPVLKGVKLHYT |
| 5 | HA-Tag nucleic | TACCCATACGATGTTCCAGATTACGCT |
| | acid sequence | |
| 6 | HA-Tag amino | YPYDVPDYA |
| | acid sequence | |
| 7 | SARS2 Spike | ATGTTTGTTTTTCTTGTTTTATTGCCACTAGTCTCTAGTCAGTGTGTTAAT |
| | Protein Open | CTTACAACCAGAACTCAATTACCCCCTGCATACACTAATTCTTTCACACGT |
| | Reading Frame | GGTGTTTATTACCCTGACAAAGTTTTCAGATCCTCAGTTTTACATTCAACT |
| | Sequence from | CAGGACTTGTTCTTACCTTTCTTTTCCAATGTTACTTGGTTCCATGCTATA |
| | SEQ ID NO: 3 | CATGTCTCTGGGACCAATGGTACTAAGAGGTTTGATAACCCTGTCCTACCA |
| | | TTTAATGATGGTGTTTATTTTGCTTCCACTGAGAAGTCTAACATAATAAGA |
| | | GGCTGGATTTTTGGTACTACTTTAGATTCGAAGACCCAGTCCCTACTTATT |
| | | GTTAATAACGCTACTAATGTTGTTATTAAAGTCTGTGAATTTCAATTTTGT |
| | | AATGATCCATTTTTGGGTGTTTATTACCACAAAAACAACAAAAGTTGGATG |
| | | GAAAGTGAGTTCAGAGTTTATTCTAGTGCGAATAATTGCACTTTTGAATAT |
| | | GTCTCTCAGCCTTTTCTTATGGACCTTGAAGGAAAACAGGGTAATTTCAAA |
| | | AATCTTAGGGAATTTGTGTTTAAGAATATTGATGGTTATTTTAAAATATAT |
| | | TCTAAGCACACGCCTATTAATTTAGTGCGTGATCTCCCTCAGGGTTTTTCG |
| | | GCTTTAGAACCATTGGTAGATTTGCCAATAGGTATTAACATCACTAGGTTT |
| | | CAAACTTTACTTGCTTTACATAGAAGTTATTTGACTCCTGGTGATTCTTCT |
| | | TCAGGTTGGACAGCTGGTGCTGCAGCTTATTATGTGGGTTATCTTCAACCT |
| | | AGGACTTTTCTATTAAAATATAATGAAAATGGAACCATTACAGATGCTGTA |
| | | GACTGTGCACTTGACCCTCTCTCAGAAACAAAGTGTACGTTGAAATCCTTC |
| | | ACTGTAGAAAAAGGAATCTATCAAACTTCTAACTTTAGAGTCCAACCAACA |
| | | GAATCTATTGTTAGATTTCCTAATATTACAAACTTGTGCCCTTTTGGTGAA |
| | | GTTTTTAACGCCACCAGATTTGCATCTGTTTATGCTTGGAACAGGAAGAGA |
| | | ATCAGCAACTGTGTTGCTGATTATTCTGTCCTATATAATTCCGCATCATTT |
| | | TCCACTTTTAAGTGTTATGGAGTGTCTCCTACTAAATTAAATGATCTCTGC |
| | | TTTACTAATGTCTATGCAGATTCATTTGTAATTAGAGGTGATGAAGTCAGA |
| | | CAAATCGCTCCAGGGCAAACTGGAAAGATTGCTGATTATAATTATAAATTA |
| | | CCAGATGATTTTACAGGCTGCGTTATAGCTTGGAATTCTAACAATCTTGAT |
| | | TCTAAGGTTGGTGGTAATTATAATTACCTGTATAGATTGTTTAGGAAGTCT |
| | | AATCTCAAACCTTTTGAGAGAGATATTTCAACTGAAATCTATCAGGCCGGT |
| | | AGCACACCTTGTAATGGTGTTGAAGGTTTTAATTGTTACTTTCCTTTACAA |
| | | TCATATGGTTTCCAACCCACTAATGGTGTTGGTTACCAACCATACAGAGTA |
| | | GTAGTACTTTCTTTTGAACTTCTACATGCACCAGCAACTGTTTGTGGACCT |
| | | AAAAAGTCTACTAATTTGGTTAAAAACAAATGTGTCAATTTCAACTTCAAT |
| | | GGTTTAACAGGCACAGGTGTTCTTACTGAGTCTAACAAAAAGTTTCTGCCT |
| | | TTCCAACAATTTGGCAGAGACATTGCTGACACTACTGATGCTGTCCGTGAT |
| | | CCACAGACACTTGAGATTCTTGACATTACACCATGTTCTTTTGGTGGTGTC |
| | | AGTGTTATAACACCAGGAACAAATACTTCTAACCAGGTTGCTGTTCTTTAT |
| | | CAGGATGTTAACTGCACAGAAGTCCCTGTTGCTATTCATGCAGATCAACTT |
| | | ACTCCTACTTGGCGTGTTTATTCTACAGGTTCTAATGTTTTTCAAACACGT |
| | | GCAGGCTGTTTAATAGGGGCTGAACATGTCAACAACTCATATGAGTGTGAC |
| | | ATACCCATTGGTGCAGGTATATGCGCTAGTTATCAGACTCAGACTAATTCT |
| | | CCTCGGCGGGCACGTAGTGTAGCTAGTCAATCCATCATTGCCTACACTATG |
| | | TCACTTGGTGCAGAAAATTCAGTTGCTTACTCTAATAACTCTATTGCCATA |
| | | CCCACAAATTTTACTATTAGTGTTACCACAGAAATTCTACCAGTGTCTATG |
| | | ACCAAGACATCAGTAGATTGTACAATGTACATTTGTGGTGATTCAACTGAA |
| | | TGCAGCAATCTTTTGTTGCAATATGGCAGTTTTTGTACACAATTAAACCGT |
| | | GCTTTAACTGGAATAGCTGTTGAACAAGACAAAAACACCCAAGAAGTTTTT |
| | | GCACAAGTCAAACAAATTTACAAAACACCACCAATTAAAGATTTTGGTGGT |
| | | TTTAATTTTTCACAAATATTACCAGATCCATCAAAACCAAGCAAGAGGTCA |
| | | TTTATTGAAGATCTACTTTTCAACAAAGTGACACTTGCAGATGCTGGCTTC |
| | | ATCAAACAATATGGTGATTGCCTTGGTGATATTGCTGCTAGAGACCTCATT |
| | | TGTGCACAAAAGTTTAACGGCCTTACTGTTTTGCCACCTTTGCTCACAGAT |
| | | GAAATGATTGCTCAATACACTTCTGCACTGTTAGCGGGTACAATCACTTCT |
| | | GGTTGGACCTTTGGTGCAGGTGCTGCATTACAAATACCATTTGCTATGCAA |
| | | ATGGCTTATAGGTTTAATGGTATTGGAGTTACACAGAATGTTCTCTATGAG |
| | | AACCAAAAATTGATTGCCAACCAATTTAATAGTGCTATTGGCAAAATTCAA |
| | | GACTCACTTTCTTCCACAGCAAGTGCACTTGGAAAACTTCAAGATGTGGTC |
| | | AACCAAAATGCACAAGCTTTAAACACGCTTGTTAAACAACTTAGCTCCAAT |
| | | TTTGGTGCAATTTCAAGTGTTTTAAATGATATCCTTTCACGTCTTGACAAA |
| | | GTTGAGGCTGAAGTGCAAATTGATAGGTTGATCACAGGCAGACTTCAAAGT |
| | | TTGCAGACATATGTGACTCAACAATTAATTAGAGCTGCAGAAATCAGAGCT |
| | | TCTGCTAATCTTGCTGCTACTAAAATGTCAGAGTGTGTACTTGGACAATCA |
| | | AAAAGAGTTGATTTTTGTGGAAAGGGCTATCATCTTATGTCCTTCCCTCAG |
| | | TCAGCACCTCATGGTGTAGTCTTCTTGCATGTGACTTATGTCCCTGCACAA |
| | | GAAAAGAACTTCACAACTGCTCCTGCCATTTGTCATGATGGAAAAGCACAC |
| | | TTTCCTCGTGAAGGTGTCTTTGTTTCAAATGGCACACACTGGTTTGTAACA |
| | | CAAAGGAATTTTTATGAACCACAAATCATTACTACAGACAACACATTTGTG |
| | | TCTGGTAACTGTGATGTTGTAATAGGAATTGTCAACAACACAGTTTATGAT |
| | | CCTTTGCAACCTGAATTAGACTCATTCAAGGAGGAGTTAGATAAATATTTT |
| | | AAGAATCATACATCACCAGATGTTGATTTAGGTGACATCTCTGGCATTAAT |
| | | GCTTCAGTTGTAAACATTCAAAAAGAAATTGACCGCCTCAATGAGGTTGCC |
| | | AAGAATTTAAATGAATCTCTCATCGATCTCCAAGAACTTGGAAAGTATGAG |
| | | CAGTATATAAAATGGCCATGGTACATTTGGCTAGGTTTTATAGCTGGCTTG |
| | | ATTGCCATAGTAATGGTGACAATTATGCTTTGCTGTATGACCAGTTGCTGT |
| | | AGTTGTCTCAAGGGCTGTTGTTCTTGTGGATCCTGCTGCAAATTTGATGAA |
| | | GACGACTCTGAGCCAGTGCTCAAAGGAGTCAAATTACATTACACA |
| 8 | Lentiviral | GACGGATCGGGAGATCTCCCGATCCCCTATGGTCGACTCTCAGTACAATCT |
| | expression vector | GCTCTGATGCCGCATAGTTAAGCCAGTATCTGCTCCCTGCTTGTGTGTTGG |
| | HIV pBOB-CAG | AGGTCGCTGAGTAGTGCGCGAGCAAAATTTAAGCTACAACAAGGCAAGGCT |
| | | TGACCGACAATTGCATGAAGAATCTGCTTAGGGTTAGGCGTTTTGCGCTGC |
| | | TTCGCGATGTACGGGCCAGATATACGCGTTGACATTGATTATTGACTAGTT |
| | | ATTAATAGTAATCAATTACGGGGTCATTAGTTCATAGCCCATATATGGAGT |
| | | TCCGCGTTACATAACTTACGGTAAATGGCCCGCCTGGCTGACCGCCCAACG |
| | | ACCCCCGCCCATTGACGTCAATAATGACGTATGTTCCCATAGTAACGCCAA |
| | | TAGGGACTTTCCATTGACGTCAATGGGTGGACTATTTACGGTAAACTGCCC |
| | | ACTTGGCAGTACATCAAGTGTATCATATGCCAAGTACGCCCCCTATTGACG |
| | | TCAATGACGGTAAATGGCCCGCCTGGCATTATGCCCAGTACATGACCTTAT |
| | | GGGACTTTCCTACTTGGCAGTACATCTACGTATTAGTCATCGCTATTACCA |
| | | TGGTGATGCGGTTTTGGCAGTACATCAATGGGCGTGGATAGCGGTTTGACT |
| | | CACGGGGATTTCCAAGTCTCCACCCCATTGACGTCAATGGGAGTTTGTTTT |
| | | GGCACCAAAATCAACGGGACTTTCCAAAATGTCGTAACAACTCCGCCCCAT |
| | | TGACGCAAATGGGCGGTAGGCGTGTACGGTGGGAGGTCTATATAAGCAGCG |
| | | CGTTTTGCCTGTACTGGGTCTCTCTGGTTAGACCAGATCTGAGCCTGGGAG |
| | | CTCTCTGGCTAACTAGGGAACCCACTGCTTAAGCCTCAATAAAGCTTGCCT |
| | | TGAGTGCTTCAAGTAGTGTGTGCCCGTCTGTTGTGTGACTCTGGTAACTAG |
| | | AGATCCCTCAGACCCTTTTAGTCAGTGTGGAAAATCTCTAGCAGTGGCGCC |
| | | CGAACAGGGACTTGAAAGCGAAAGGGAAACCAGAGGAGCTCTCTCGACGCA |
| | | GGACTCGGCTTGCTGAAGCGCGCACGGCAAGAGGCGAGGGGCGGCGACTGG |
| | | TGAGTACGCCAAAAATTTTGACTAGCGGAGGCTAGAAGGAGAGAGATGGGT |
| | | GCGAGAGCGTCAGTATTAAGCGGGGGAGAATTAGATCGCGATGGGAAAAAA |
| | | TTCGGTTAAGGCCAGGGGGAAAGAAAAAATATAAATTAAAACATATAGTAT |
| | | GGGCAAGCAGGGAGCTAGAACGATTCGCAGTTAATCCTGGCCTGTTAGAAA |
| | | CATCAGAAGGCTGTAGACAAATACTGGGACAGCTACAACCATCCCTTCAGA |
| | | CAGGATCAGAAGAACTTAGATCATTATATAATACAGTAGCAACCCTCTATT |
| | | GTGTGCATCAAAGGATAGAGATAAAAGACACCAAGGAAGCTTTAGACAAGA |
| | | TAGAGGAAGAGCAAAACAAAAGTAAGACCACCGCACAGCAAGCGGCCGCTG |
| | | ATCTTCAGACCTGGAGGAGGAGATATGAGGGACAATTGGAGAAGTGAATTA |
| | | TATAAATATAAAGTAGTAAAAATTGAACCATTAGGAGTAGCACCCACCAAG |
| | | GCAAAGAGAAGAGTGGTGCAGAGAGAAAAAAGAGCAGTGGGAATAGGAGCT |
| | | TTGTTCCTTGGGTTCTTGGGAGCAGCAGGAAGCACTATGGGCGCAGCCTCA |
| | | ATGACGCTGACGGTACAGGCCAGACAATTATTGTCTGGTATAGTGCAGCAG |
| | | CAGAACAATTTGCTGAGGGCTATTGAGGCGCAACAGCATCTGTTGCAACTC |
| | | ACAGTCTGGGGCATCAAGCAGCTCCAGGCAAGAATCCTGGCTGTGGAAAGA |
| | | TACCTAAAGGATCAACAGCTCCTGGGGATTTGGGGTTGCTCTGGAAAACTC |
| | | ATTTGCACCACTGCTGTGCCTTGGAATGCTAGTTGGAGTAATAAATCTCTG |
| | | GAACAGATTTGGAATCACACGACCTGGATGGAGTGGGACAGAGAAATTAAC |
| | | AATTACACAAGCTTAATACACTCCTTAATTGAAGAATCGCAAAACCAGCAA |
| | | GAAAAGAATGAACAAGAATTATTGGAATTAGATAAATGGGCAAGTTTGTGG |
| | | AATTGGTTTAACATAACAAATTGGCTGTGGTATATAAAATTATTCATAATG |
| | | ATAGTAGGAGGCTTGGTAGGTTTAAGAATAGTTTTTGCTGTACTTTCTATA |
| | | GTGAATAGAGTTAGGCAGGGATATTCACCATTATCGTTTCAGACCCACCTC |
| | | CCAACCCCGAGGGGACCCGACAGGCCCGAAGGAATAGAAGAAGAAGGTGGA |
| | | GAGAGAGACAGAGACAGATCCATTCGATTAGTGAACGGATCAACTTTTAAA |
| | | AGAAAAGGGGGGATTGGGGGGTACAGTGCAGGGGAAAGAATAGTAGACATA |
| | | ATAGCAACAGACATACAAACTAAAGAATTACAAAAACAAATTACAAAATTC |
| | | AAAATTTTATCGATGTCGACATTGATTATTGACTAGTTATTAATAGTAATC |
| | | AATTACGGGGTCATTAGTTCATAGCCCATATATGGAGTTCCGCGTTACATA |
| | | ACTTACGGTAAATGGCCCGCCTGGCTGACCGCCCAACGACCCCCGCCCATT |
| | | GACGTCAATAATGACGTATGTTCCCATAGTAACGCCAATAGGGACTTTCCA |
| | | TTGACGTCAATGGGTGGAGTATTTACGGTAAACTGCCCACTTGGCAGTACA |
| | | TCAAGTGTATCATATGCCAAGTACGCCCCCTATTGACGTCAATGACGGTAA |
| | | ATGGCCCGCCTGGCATTATGCCCAGTACATGACCTTATGGGACTTTCCTAC |
| | | TTGGCAGTACATCTACGTATTAGTCATCGCTATTACCATGGTCGAGGTGAG |
| | | CCCCACGTTCTGCTTCACTCTCCCCATCTCCCCCCCCTCCCCACCCCCAAT |
| | | TTTGTATTTATTTATTTTTTAATTATTTTGTGCAGCGATGGGGGCGGGGGG |
| | | GGGGGGGGCGCGCGCCAGGCGGGGCGGGGCGGGGCGAGGGGCGGGGCGGGG |
| | | CGAGGCGGAGAGGTGCGGCGGCAGCCAATCAGAGCGGCGCGCTCCGAAAGT |
| | | TTCCTTTTATGGCGAGGCGGCGGCGGCGGCGGCCCTATAAAAAGCGAAGCG |
| | | CGCGGCGGGCGGGAGTCGCTGCGTTGCCTTCGCCCCGTGCCCCGCTCCGCG |
| | | CCGCCTCGCGCCGCCCGCCCCGGCTCTGACTGACCGCGTTACTCCCACAGG |
| | | TGAGCGGGCGGGACGGCCCTTCTCCTCCGGGCTGTAATTAGCGCTTGGTTT |
| | | AATGACGGCTCGTTTCTTTTCTGTGGCTGCGTGAAAGCCTTAAAGGGCTCC |
| | | GGGAGGGCCCTTTGTGCGGGGGGGAGCGGCTCGGGGGGTGCGTGCGTGTGT |
| | | GTGTGCGTGGGGAGCGCCGCGTGCGGCCCGCGCTGCCCGGCGGCTGTGAGC |
| | | GCTGCGGGCGCGGCGCGGGGCTTTGTGCGCTCCGCGTGTGCGCGAGGGGAG |
| | | CGCGGCCGGGGGCGGTGCCCCGCGGTGCGGGGGGGCTGCGAGGGGAACAAA |
| | | GGCTGCGTGCGGGGTGTGTGCGTGGGGGGGTGAGCAGGGGGTGTGGGCGCG |
| | | GCGGTCGGGCTGTAACCCCCCCCTGCACCCCCCTCCCCGAGTTGCTGAGCA |
| | | CGGCCCGGCTTCGGGTGCGGGGCTCCGTGCGGGGCGTGGCGCGGGGCTCGC |
| | | CGTGCCGGGCGGGGGGTGGCGGCAGGTGGGGGTGCCGGGCGGGGCGGGGCC |
| | | GCCTCGGGCCGGGGAGGGCTCGGGGGAGGGGCGCGGCGGCCCCGGAGCGCC |
| | | GGCGGCTGTCGAGGCGCGGCGAGCCGCAGCCATTGCCTTTTATGGTAATCG |
| | | TGCGAGAGGGCGCAGGGACTTCCTTTGTCCCAAATCTGTCGGAGCCGAAAT |
| | | CTGGGAGGCGCCGCCGCACCCCCTCTAGCGGGCGCGGGGCGAAGCGGTGCG |
| | | GCGCCGGCAGGAAGGAAATGGGCGGGGAGGGCCTTCGTGCGTCGCCGCGCC |
| | | GCCGTCCCCTTCTCCCTCTCCAGCCTCGGGGCTGTCCGCGGGGGGACGGCT |
| | | GCCTTCGGGGGGGACGGGGCAGGGCGGGGTTCGGCTTCTGGCGTGTGACCG |
| | | GCGGCTCTAGAGCCTCTGCTAACCATGTTCATGCCTTCTTCTTTTTCCTAC |
| | | AGCTCCTGGGCAACGTGCTGGTTATTGTGCTGTCTCATCATTTTGGCAAAG |
| | | AATTCGCTAGTGGATCCACCGGTGGCCGCCTGGGCCCGTTAACGTCTCGAG |
| | | TTTAAACCTGGAAAAACATGGAGCAATCACAAGTAGCAATACAGCAGCTAC |
| | | CAATGCTGATTGTGCCTGGCTAGAAGCACAAGAGGAGGAGGAGGTGGGTTT |
| | | TCCAGTCACACCTCAGACAATCAACCTCTGGATTACAAAATTTGTGAAAGA |
| | | TTGACTGGTATTCTTAACTATGTTGCTCCTTTTACGCTATGTGGATACGCT |
| | | GCTTTAATGCCTTTGTATCATGCTATTGCTTCCCGTATGGCTTTCATTTTC |
| | | TCCTCCTTGTATAAATCCTGGTTGCTGTCTCTTTATGAGGAGTTGTGGCCC |
| | | GTTGTCAGGCAACGTGGCGTGGTGTGCACTGTGTTTGCTGACGCAACCCCC |
| | | ACTGGTTGGGGCATTGCCACCACCTGTCAGCTCCTTTCCGGGACTTTCGCT |
| | | TTCCCCCTCCCTATTGCCACGGCGGAACTCATCGCCGCCTGCCTTGCCCGC |
| | | TGCTGGACAGGGGCTCGGCTGTTGGGCACTGACAATTCCGTGGTGTTGTCG |
| | | GGGAAGCTGACGTCCTTTCCATGGCTGCTCGCCTGTGTTGCCACCTGGATT |
| | | CTGCGCGGGACGTCCTTCTGCTACGTCCCTTCGGCCCTCAATCCAGCGGAC |
| | | CTTCCTTCCCGCGGCCTGCTGCCGGCTCTGCGGCCTCTTCCGCGTCTTCGC |
| | | CTTCGCCCTCAGACGAGTCGGATCTCCCTTTGGGCCGCCTCCCCGCCTGGA |
| | | ATTCGAGCTCGGTACCTTTAAGACCAATGACTTACAAGGCAGCTGTAGATC |
| | | TTAGCCACTTTTTAAAAGAAAAGGGGGGACTGGAAGGGCTAATTCACTCCC |
| | | AACGAAGACAAGATATCCTTGATCTGTGGATCTACCACACACAAGGCTACT |
| | | TCCCTGATTGGCAGAACTACACACCAGGGCCAGGGATCAGATATCCACTGA |
| | | CCTTTGGATGGTGCTACAAGCTAGTACCAGTTGAGCAAGAGAAGGTAGAAG |
| | | AAGCCAATGAAGGAGAGAACACCCGCTTGTTACACCCTGTGAGCCTGCATG |
| | | GGATGGATGACCCGGAGAGAGAAGTATTAGAGTGGAGGTTTGACAGCCGCC |
| | | TAGCATTTCATCACATGGCCCGAGAGCTGCATCCGGACTGTACTGGGTCTC |
| | | TCTGGTTAGACCAGATCTGAGCCTGGGAGCTCTCTGGCTAACTAGGGAACC |
| | | CACTGCTTAAGCCTCAATAAAGCTTGCCTTGAGTGCTTCAAGTAGTGTGTG |
| | | CCCGTCTGTTGTGTGACTCTGGTAACTAGAGATCCCTCAGACCCTTTTAGT |
| | | CAGTGTGGAAAATCTCTAGCAGGGCCGCTTTAAACCCGCTGATCAGCCTCG |
| | | ACTGTGCCTTCTAGTTGCCAGCCATCTGTTGTTTGCCCCTCCCCCGTGCCT |
| | | TCCTTGACCCTGGAAGGTGCCACTCCCACTGTCCTTTCCTAATAAAATGAG |
| | | GAAATTGCATCGCATTGTCTGAGTAGGTGTCATTCTATTCTGGGGGGTGGG |
| | | GTGGGGCAGGACAGCAAGGGGGAGGATTGGGAAGACAATAGCAGGCATGCT |
| | | GGGGATGCGGTGGGCTCTATGGCTTCTGAGGCGGAAAGAACCAGCTGGGGC |
| | | TCTAGGGGGTATCCCCACGCGCCCTGTAGCGGCGCATTAAGCGCGGCGGGT |
| | | GTGGTGGTTACGCGCAGCGTGACCGCTACACTTGCCAGCGCCCTAGCGCCC |
| | | GCTCCTTTCGCTTTCTTCCCTTCCTTTCTCGCCACGTTCGCCGGCTTTCCC |
| | | CGTCAAGCTCTAAATCGGGGGCTCCCTTTAGGGTTCCGATTTAGTGCTTTA |
| | | CGGCACCTCGACCCCAAAAAACTTGATTAGGGTGATGGTTCACGTAGTGGG |
| | | CCATCGCCCTGATAGACGGTTTTTCGCCCTTTGACGTTGGAGTCCACGTTC |
| | | TTTAATAGTGGACTCTTGTTCCAAACTGGAACAACACTCAACCCTATCTCG |
| | | GTCTATTCTTTTGATTTATAAGGGATTTTGGGGATTTCGGCCTATTGGTTA |
| | | AAAAATGAGCTGATTTAACAAAAATTTAACGCGAATTAATTCTGTGGAATG |
| | | TGTGTCAGTTAGGGTGTGGAAAGTCCCCAGGCTCCCCAGCAGGCAGAAGTA |
| | | TGCAAAGCATGCATCTCAATTAGTCAGCAACCAGGTGTGGAAAGTCCCCAG |
| | | GCTCCCCAGCAGGCAGAAGTATGCAAAGCATGCATCTCAATTAGTCAGCAA |
| | | CCATAGTCCCGCCCCTAACTCCGCCCATCCCGCCCCTAACTCCGCCCAGTT |
| | | CCGCCCATTCTCCGCCCCATGGCTGACTAATTTTTTTTATTTATGCAGAGG |
| | | CCGAGGCCGCCTCTGCCTCTGAGCTATTCCAGAAGTAGTGAGGAGGCTTTT |
| | | TTGGAGGCCTAGGCTTTTGCAAAAAGCTCCCGGGAGCTTGTATATCCATTT |
| | | TCGGATCTGATCAGCACGTGTTGACAATTAATCATCGGCATAGTATATCGG |
| | | CATAGTATAATACGACAAGGTGAGGAACTAAACCATGGCCAAGTTGACCAG |
| | | TGCCGTTCCGGTGCTCACCGCGCGCGACGTCGCCGGAGCGGTCGAGTTCTG |
| | | GACCGACCGGCTCGGGTTCTCCCGGGACTTCGTGGAGGACGACTTCGCCGG |
| | | TGTGGTCCGGGACGACGTGACCCTGTTCATCAGCGCGGTCCAGGACCAGGT |
| | | GGTGCCGGACAACACCCTGGCCTGGGTGTGGGTGCGCGGCCTGGACGAGCT |
| | | GTACGCCGAGTGGTCGGAGGTCGTGTCCACGAACTTCCGGGACGCCTCCGG |
| | | GCCGGCCATGACCGAGATCGGCGAGCAGCCGTGGGGGCGGGAGTTCGCCCT |
| | | GCGCGACCCGGCCGGCAACTGCGTGCACTTCGTGGCCGAGGAGCAGGACTG |
| | | ACACGTGCTACGAGATTTCGATTCCACCGCCGCCTTCTATGAAAGGTTGGG |
| | | CTTCGGAATCGTTTTCCGGGACGCCGGCTGGATGATCCTCCAGCGCGGGGA |
| | | TCTCATGCTGGAGTTCTTCGCCCACCCCAACTTGTTTATTGCAGCTTATAA |
| | | TGGTTACAAATAAAGCAATAGCATCACAAATTTCACAAATAAAGCATTTTT |
| | | TTCACTGCATTCTAGTTGTGGTTTGTCCAAACTCATCAATGTATCTTATCA |
| | | TGTCTGTATACCGTCGACCTCTAGCTAGAGCTTGGCGTAATCATGGTCATA |
| | | GCTGTTTCCTGTGTGAAATTGTTATCCGCTCACAATTCCACACAACATACG |
| | | AGCCGGAAGCATAAAGTGTAAAGCCTGGGGTGCCTAATGAGTGAGCTAACT |
| | | CACATTAATTGCGTTGCGCTCACTGCCCGCTTTCCAGTCGGGAAACCTGTC |
| | | GTGCCAGCTGCATTAATGAATCGGCCAACGCGCGGGGAGAGGCGGTTTGCG |
| | | TATTGGGCGCTCTTCCGCTTCCTCGCTCACTGACTCGCTGCGCTCGGTCGT |
| | | TCGGCTGCGGCGAGCGGTATCAGCTCACTCAAAGGCGGTAATACGGTTATC |
| | | CACAGAATCAGGGGATAACGCAGGAAAGAACATGTGAGCAAAAGGCCAGCA |
| | | AAAGGCCAGGAACCGTAAAAAGGCCGCGTTGCTGGCGTTTTTCCATAGGCT |
| | | CCGCCCCCCTGACGAGCATCACAAAAATCGACGCTCAAGTCAGAGGTGGCG |
| | | AAACCCGACAGGACTATAAAGATACCAGGCGTTTCCCCCTGGAAGCTCCCT |
| | | CGTGCGCTCTCCTGTTCCGACCCTGCCGCTTACCGGATACCTGTCCGCCTT |
| | | TCTCCCTTCGGGAAGCGTGGCGCTTTCTCATAGCTCACGCTGTAGGTATCT |
| | | CAGTTCGGTGTAGGTCGTTCGCTCCAAGCTGGGCTGTGTGCACGAACCCCC |
| | | CGTTCAGCCCGACCGCTGCGCCTTATCCGGTAACTATCGTCTTGAGTCCAA |
| | | CCCGGTAAGACACGACTTATCGCCACTGGCAGCAGCCACTGGTAACAGGAT |
| | | TAGCAGAGCGAGGTATGTAGGCGGTGCTACAGAGTTCTTGAAGTGGTGGCC |
| | | TAACTACGGCTACACTAGAAGAACAGTATTTGGTATCTGCGCTCTGCTGAA |
| | | GCCAGTTACCTTCGGAAAAAGAGTTGGTAGCTCTTGATCCGGCAAACAAAC |
| | | CACCGCTGGTAGCGGTGGTTTTTTTGTTTGCAAGCAGCAGATTACGCGCAG |
| | | AAAAAAAGGATCTCAAGAAGATCCTTTGATCTTTTCTACGGGGTCTGACGC |
| | | TCAGTGGAACGAAAACTCACGTTAAGGGATTTTGGTCATGAGATTATCAAA |
| | | AAGGATCTTCACCTAGATCCTTTTAAATTAAAAATGAAGTTTTAAATCAAT |
| | | CTAAAGTATATATGAGTAAACTTGGTCTGACAGTTACCAATGCTTAATCAG |
| | | TGAGGCACCTATCTCAGCGATCTGTCTATTTCGTTCATCCATAGTTGCCTG |
| | | ACTCCCCGTCGTGTAGATAACTACGATACGGGAGGGCTTACCATCTGGCCC |
| | | CAGTGCTGCAATGATACCGCGAGACCCACGCTCACCGGCTCCAGATTTATC |
| | | AGCAATAAACCAGCCAGCCGGAAGGGCCGAGCGCAGAAGTGGTCCTGCAAC |
| | | TTTATCCGCCTCCATCCAGTCTATTAATTGTTGCCGGGAAGCTAGAGTAAG |
| | | TAGTTCGCCAGTTAATAGTTTGCGCAACGTTGTTGCCATTGCTACAGGCAT |
| | | CGTGGTGTCACGCTCGTCGTTTGGTATGGCTTCATTCAGCTCCGGTTCCCA |
| | | ACGATCAAGGCGAGTTACATGATCCCCCATGTTGTGCAAAAAAGCGGTTAG |
| | | CTCCTTCGGTCCTCCGATCGTTGTCAGAAGTAAGTTGGCCGCAGTGTTATC |
| | | ACTCATGGTTATGGCAGCACTGCATAATTCTCTTACTGTCATGCCATCCGT |
| | | AAGATGCTTTTCTGTGACTGGTGAGTACTCAACCAAGTCATTCTGAGAATA |
| | | GTGTATGCGGCGACCGAGTTGCTCTTGCCCGGCGTCAATACGGGATAATAC |
| | | CGCGCCACATAGCAGAACTTTAAAAGTGCTCATCATTGGAAAACGTTCTTC |
| | | GGGGCGAAAACTCTCAAGGATCTTACCGCTGTTGAGATCCAGTTCGATGTA |
| | | ACCCACTCGTGCACCCAACTGATCTTCAGCATCTTTTACTTTCACCAGCGT |
| | | TTCTGGGTGAGCAAAAACAGGAAGGCAAAATGCCGCAAAAAAGGGAATAAG |
| | | GGCGACACGGAAATGTTGAATACTCATACTCTTCCTTTTTCAATATTATTG |
| | | AAGCATTTATCAGGGTTATTGTCTCATGAGCGGATACATATTTGAATGTAT |
| | | TTAGAAAAATAAACAAATAGGGGTTCCGCGCACATTTCCCCGAAAAGTGCC |
| | | ACCTGACGTC |
| 9 | SARS-CoV-2 | ATGTTCGTATTCCTTGTCCTCCTCCCCCTCGTCAGCTCCCAGTGTGTCAAT |
| | codon optimized | CTGACAACTCGAACTCAGCTCCCACCCGCATACACCAATAGTTTTACCCGA |
| | sequence | GGAGTCTATTATCCCGACAAAGTTTTCAGgTCTAGTGTGTTGCATAGCACG |
| | | CAGGACCTGTTCTTGCCGTTTTTTTCAAATGTGACATGGTTCCATGCCATC |
| | | CATGTCTCTGGTACGAACGGGACCAAAAGGTTTGATAACCCCGTTCTTCCA |
| | | TTTAACGACGGGGTTTATTTCGCCTCTACTGAAAAGTCTAATATAATACGA |
| | | GGTTGGATTTTTGGGACAACACTGGACTCTAAAACGCAATCTCTCCTCATA |
| | | GTAAATAATGCGACGAATGTCGTAATCAAAGTATGTGAGTTTCAGTTTTGC |
| | | AACGACCCTTTTCTTGGTGTATACTATCACAAGAACAACAAGTCTTGGATG |
| | | GAAAGTGAATTtAGAGTCTACAGCTCCGCAAACAACTGTACCTTTGAGTAC |
| | | GTCAGTCAGCCCTTCCTTATGGACCTCGAAGGCAAACAGGGGAACTTCAAG |
| | | AATCTGCGCGAGTTCGTGTTCAAGAATATCGACGGCTATTTCAAGATATAT |
| | | TCAAAACATACACCAATCAATTTGGTACGCGATTTGCCGCAGGGCTTCTCT |
| | | GCTCTTGAGCCTCTCGTAGATTTGCCTATAGGGATCAATATTACTCGCTTT |
| | | CAAACTCTCCTTGCCCTTCATCGGTCCTACCTGACACCAGGCGACAGCTCC |
| | | AGTGGGTGGACAGCTGGTGCAGCGGCTTACTACGTCGGCTACCTCCAACCC |
| | | AGGACTTTTTTGCTGAAATATAATGAGAATGGTACGATTACTGACGCCGTC |
| | | GATTGTGCCCTCGACCCATTGAGCGAAACTAAATGCACCCTGAAAAGCTTT |
| | | ACGGTTGAGAAAGGGATATATCAGACATCTAATTTTCGCGTTCAACCCACG |
| | | GAATCCATTGTACGATTTCCCAACATTACCAATTTGTGTCCTTTTGGTGAG |
| | | GTATTTAATGCAACCAGATTCGCGAGTGTGTATGCCTGGAATAGGAAAAGA |
| | | ATTTCTAACTGTGTAGCAGACTACAGCGTTCTGTATAATAGTGCGTCCTTT |
| | | AGTACGTTCAAGTGCTACGGAGTAAGCCCGACAAAGTTGAACGATTTGTGT |
| | | TTCACTAACGTCTATGCCGATAGCTTCGTCATCAGGGGAGACGAAGTAAGG |
| | | CAAATTGCACCAGGCCAGACCGGaAAAATAGCCGACTACAACTATAAGTTG |
| | | CCGGACGATTTCACCGGGTGTGTCATCGCGTGGAATAGTAATAACCTGGAC |
| | | TCCAAGGTGGGCGGCAACTACAACTACCTGTACCGATTGTTCCGCAAGTCT |
| | | AACCTCAAGCCCTTTGAACGAGACATATCCACCGAGATATACCAAGCGGGA |
| | | AGCACTCCCTGTAATGGCGTTGAAGGTTTCAACTGCTATTTTCCTCTCCAG |
| | | TCCTATGGCTTCCAACCTACCAATGGAGTAGGCTATCAACCGTATCGAGTC |
| | | GTGGTGCTGAGTTTCGAGCTCCTGCACGCTCCAGCGACCGTTTGTGGTCCT |
| | | AAGAAAAGTACAAACCTCGTAAAAAATAAGTGCGTAAACTTTAACTTTAAC |
| | | GGTCTCACAGGAACGGGAGTTCTTACAGAGTCCAATAAGAAGTTCCTGCCC |
| | | TTTCAGCAGTTTGGGCGCGACATCGCGGACACCACAGATGCAGTCAGGGAC |
| | | CCGCAGACTCTTGAGATCCTGGAcATCACCCCATGCTCTTTTGGCGGTGTG |
| | | AGTGTCATTACACCCGGGACCAACACCAGCAATCAGGTTGCTGTGCTCTAT |
| | | CAAGACGTCAACTGTACGGAAGTCCCAGTTGCAATACACGCGGACCAGTTG |
| | | ACGCCGACGTGGCGGGTTTATTCTACCGGCTCAAACGTCTTTCAAACTAGA |
| | | GCCGGTTGTTTGATCGGGGCGGAGCATGTGAACAATTCATACGAATGTGAT |
| | | ATTCCCATAGGAGCCGGAATATGTGCTTCTTACCAGACCCAAACCAACTCT |
| | | CCGAGACGAGCCCGGTCCGTAGCCAGTCAAAGCATAATTGCGTACACCATG |
| | | AGCCTCGGTGCAGAAAACTCAGTTGCGTACTCAAATAACTCCATTGCCATC |
| | | CCAACAAATTTTACCATATCAGTGACCACTGAAATCCTTCCAGTGAGCATG |
| | | ACTAAAACAAGTGTAGATTGCACAATGTAtATTTGCGGAGACTCAACTGAG |
| | | TGCTCTAACCTCCTGTTGCAGTACGGTTCATTCTGTACCCAGCTCAATCGC |
| | | GCACTTACGGGAATAGCAGTAGAACAAGATAAGAATACTCAGGAAGTCTTC |
| | | GCTCAAGTAAAGCAAATCTACAAGACGCCCCCCATTAAGGACTTTGGGGGT |
| | | TTTAATTTTAGCCAAATACTCCCGGAcCCCAGTAAACCCTCTAAGAGGTCA |
| | | TTTATAGAAGACCTGTTGTTTAATAAAGTTACTCTGGCCGATGCTGGCTTT |
| | | ATTAAACAATACGGTGATTGTTTGGGCGACATCGCGGCGCGGGATCTGATA |
| | | TGCGCCCAAAAATTTAACGGTCTCACTGTGTTGCCGCCTTTGCTCACAGAC |
| | | GAGATGATAGCTCAGTACACATCAGCGTTGTTGGCGGGTACCATAACGTCt |
| | | GGATGGACATTCGGAGCAGGGGCGGCCTTGCAAATACCATTCGCAATGCAA |
| | | ATGGCATACAGGTTCAACGGGATAGGTGTTACACAGAATGTACTTTACGAG |
| | | AACCAGAAATTGATTGCAAACCAGTTTAACTCTGCGATTGGCAAAATTCAG |
| | | GATAGCCTCAGCTCCACCGCGTCTGCACTGGGAAAACTCCAAGATGTTGTg |
| | | AACCAAAATGCCCAAGCCCTCAATACACTTGTGAAGCAACTCTCCAGCAAT |
| | | TTCGGTGCAATAAGTAGTGTGCTGAACGACATACTTTCTCGGTTGGACAAA |
| | | GTAGAGGCGGAGGTGCAAATCGACAGACTCATAACAGGTCGACTGCAATCA |
| | | TTGCAAACGTATGTTACTCAGCAATTGATACGGGCGGCGGAGATTCGAGCG |
| | | TCTGCCAACCTCGCCGCGACTAAAATGTCTGAGTGTGTGCTCGGTCAGTCA |
| | | AAACGCGTGGACTTCTGTGGAAAGGGATACCATCTCATGTCCTTCCCTCAG |
| | | TCTGCCCCTCACGGAGTAGTCTTTCTTCAtGTGACCTATGTTCCAGCACAG |
| | | GAGAAAAATTTTACCACAGCGCCCGCGATATGCCACGATGGCAAAGCTCAT |
| | | TTTCCGCGAGAGGGAGTATTTGTCTCTAACGGCACCCATTGGTTTGTAACC |
| | | CAGCGCAATTTTTACGAACCTCAGATAATCACCACTGACAACACCTTTGTT |
| | | AGTGGTAATTGTGATGTAGTGATCGGGATAGTgAACAATACAGTGTACGAC |
| | | CCCCTGCAACCTGAGCTGGACTCCTTTAAAGAAGAACTCGACAAGTACTTC |
| | | AAAAACCACACTTCCCCGGATGTCGATTTGGGTGATATATCTGGCATCAAT |
| | | GCGAGTGTAGTCAACATACAGAAGGAAATCGACAGGCTCAATGAAGTAGCA |
| | | AAAAACCTGAATGAATCCCTCATAGAcCTCCAAGAGCTGGGCAAATATGAG |
| | | CAGTACATTAAATGGCCCTGGTACATTTGGCTGGGCTTCATCGCTGGCCTG |
| | | ATAGCGATAGTGATGGTTACTATTATGCTCTGCTGTATGACGAGTTGTTGC |
| | | AGCTGCCTCAAAGGGTGTTGTTCTTGCGGcTCCTGTTGTAAGTTCGACGAG |
| | | GATGATTCAGAGCCCGTTCTTAAGGGTGTAAAACTCCATTACACTGGAGGA |
| | | GGAGGTTACCCATACGATGTTCCAGATTACGCT |
| 10 | HIV pBOB-CAG- | GACGGATCGGGAGATCTCCCGATCCCCTATGGTCGACTCTCAGTACAATCT |
| | SARS-CoV2 | GCTCTGATGCCGCATAGTTAAGCCAGTATCTGCTCCCTGCTTGTGTGTTGG |
| | Spike-HA with | AGGTCGCTGAGTAGTGCGCGAGCAAAATTTAAGCTACAACAAGGCAAGGCT |
| | domains | TGACCGACAATTGCATGAAGAATCTGCTTAGGGTTAGGCGTTTTGCGCTGC |
| | | TTCGCGATGTACGGGCCAGATATACGCGTTGACATTGATTATTGACTAGTT |
| | | ATTAATAGTAATCAATTACGGGGTCATTAGTTCATAGCCCATATATGGAGT |
| | | TCCGCGTTACATAACTTACGGTAAATGGCCCGCCTGGCTGACCGCCCAACG |
| | | ACCCCCGCCCATTGACGTCAATAATGACGTATGTTCCCATAGTAACGCCAA |
| | | TAGGGACTTTCCATTGACGTCAATGGGTGGACTATTTACGGTAAACTGCCC |
| | | ACTTGGCAGTACATCAAGTGTATCATATGCCAAGTACGCCCCCTATTGACG |
| | | TCAATGACGGTAAATGGCCCGCCTGGCATTATGCCCAGTACATGACCTTAT |
| | | GGGACTTTCCTACTTGGCAGTACATCTACGTATTAGTCATCGCTATTACCA |
| | | TGGTGATGCGGTTTTGGCAGTACATCAATGGGCGTGGATAGCGGTTTGACT |
| | | CACGGGGATTTCCAAGTCTCCACCCCATTGACGTCAATGGGAGTTTGTTTT |
| | | GGCACCAAAATCAACGGGACTTTCCAAAATGTCGTAACAACTCCGCCCCAT |
| | | TGACGCAAATGGGCGGTAGGCGTGTACGGTGGGAGGTCTATATAAGCAGCG |
| | | CGTTTTGCCTGTACTGGGTCTCTCTGGTTAGACCAGATCTGAGCCTGGGAG |
| | | CTCTCTGGCTAACTAGGGAACCCACTGCTTAAGCCTCAATAAAGCTTGCCT |
| | | TGAGTGCTTCAAGTAGTGTGTGCCCGTCTGTTGTGTGACTCTGGTAACTAG |
| | | AGATCCCTCAGACCCTTTTAGTCAGTGTGGAAAATCTCTAGCAGTGGCGCC |
| | | CGAACAGGGACCTGAAAGCGAAAGGGAAACCAGAGCTCTCTCGACGCAGGA |
| | | CTCGGCTTGCTGAAGCGCGCACGGCAAGAGGCGAGGGGCGGCGACTGGTGA |
| | | GTACGCCAAAAATTTTGACTAGCGGAGGCTAGAAGGAGAGAGATGGGTGCG |
| | | AGAGCGTCAGTATTAAGCGGGGGAGAATTAGATCGCGATGGGAAAAAATTC |
| | | GGTTAAGGCCAGGGGGAAAGAAAAAATATAAATTAAAACATATAGTATGGG |
| | | CAAGCAGGGAGCTAGAACGATTCGCAGTTAATCCTGGCCTGTTAGAAACAT |
| | | CAGAAGGCTGTAGACAAATACTGGGACAGCTACAACCATCCCTTCAGACAG |
| | | GATCAGAAGAACTTAGATCATTATATAATACAGTAGCAACCCTCTATTGTG |
| | | TGCATCAAAGGATAGAGATAAAAGACACCAAGGAAGCTTTAGACAAGATAG |
| | | AGGAAGAGCAAAACAAAAGTAAGACCACCGCACAGCAAGCGGCCGCTGATC |
| | | TTCAGACTTGGAGGAGGAGATATGAGGGACAATTGGAGAAGTGAATTATAT |
| | | AAATATAAAGTAGTAAAAATTGAACCATTAGGAGTAGCACCCACCAAGGCA |
| | | AAGAGAAGAGTGGTGCAGAGAGAAAAAAGAGCAGTGGGAATAGGAGCTTTG |
| | | TTCCTTGGGTTCTTGGGAGCAGCAGGAAGCACTATGGGCGCAGCCTCAATG |
| | | ACGCTGACGGTACAGGCCAGACAATTATTGTCTGGTATAGTGCAGCAGCAG |
| | | AACAATTTGCTGAGGGCTATTGAGGCGCAACAGCATCTGTTGCAACTCACA |
| | | GTCTGGGGCATCAAGCAGCTCCAAGCAAGAATCCTAGCTGTGGAAAGATAC |
| | | CTAAAGGATCAACAGCTCCTAGGGATTTGGGGTTGCTCTGGAAAACTCATT |
| | | TGCACCACTGCTGTGCCTTGGAATGCTAGTTGGAGTAATAAATCTCTGGAA |
| | | CAGATCTGGAATCACACGACCTGGATGGAGTGGGACAGAGAAATTAACAAT |
| | | TACACAAGCTTAATACACTCCTTAATTGAAGAATCGCAAAACCAGCAAGAA |
| | | AAGAATGAACAAGAATTATTGGAATTAGATAAATGGGCAAGTTTGTGGAAT |
| | | TGGTTTAACATAACAAATTGGCTGTGGTATATAAAATTATTCATAATGATA |
| | | GTAGGAGGCTTGGTAGGTTTAAGAATAGTTTTTGCTGTACTTTCTATAGTG |
| | | AATAGAGTTAGGCAGGGATATTCACCATTATCGTTTCAGACCCACCTCCCA |
| | | ATCCCGAGGGGACCCGACAGGCCCGAAGGAATAGAAGAAGAAGGTGGAGAG |
| | | AGAGACAGAGACAGATCCATTCGATTAGTGAACGGATCAACTTTTAAAAGA |
| | | AAAGGGGGGATTGGGGGGTACAGTGCAGGGGAAAGAATAGTAGACATAATA |
| | | GCAACAGACATACAAACTAAAGAATTACAAAAACAAATTACAAAAATTCAA |
| | | AATTTTATCGATGTCGACATTGATTATTGACTAGTTATTAATAGTAATCAA |
| | | TTACGGGGTCATTAGTTCATAGCCCATATATGGAGTTCCGCGTTACATAAC |
| | | TTACGGTAAATGGCCCGCCTGGCTGACCGCCCAACGACCCCCGCCCATTGA |
| | | CGTCAATAATGACGTATGTTCCCATAGTAACGCCAATAGGGACTTTCCATT |
| | | GACGTCAATGGGTGGACTATTTACGGTAAACTGCCCACTTGGCAGTACATC |
| | | AAGTGTATCATATGCCAAGTACGCCCCCTATTGACGTCAATGACGGTAAAT |
| | | GGCCCGCCTGGCATTATGCCCAGTACATGACCTTATGGGACTTTCCTACTT |
| | | GGCAGTACATCTACGTATTAGTCATCGCTATTACCATGGGTCGAGGTGAGC |
| | | CCCACGTTCTGCTTCACTCTCCCCATCTCCCCCCCCTCCCCACCCCCAATT |
| | | TTGTATTTATTTATTTTTTAATTATTTTGTGCAGCGATGGGGGCGGGGGGG |
| | | GGGGGGGCGCGCGCCAGGCGGGGCGGGGCGGGGCGAGGGGCGGGGCGGGGC |
| | | GAGGCGGAGAGGTGCGGCGGCAGCCAATCAGAGCGGCGCGCTCCGAAAGTT |
| | | TCCTTTTATGGCGAGGCGGCGGCGGCGGCGGCCCTATAAAAAGCGAAGCGC |
| | | GCGGCGGGCGGGAGTCGCTGCGTTGCCTTCGCCCCGTGCCCCGCTCCGCGC |
| | | CGCCTCGCGCCGCCCGCCCCGGCTCTGACTGACCGCGTTACTCCCACAGGT |
| | | GAGCGGGCGGGACGGCCCTTCTCCTCCGGGCTGTAATTAGCGCTTGGTTTA |
| | | ATGACGGCTCGTTTCTTTTCTGTGGCTGCGTGAAAGCCTTAAAGGGCTCCG |
| | | GGAGGGCCCTTTGTGCGGGGGGGAGCGGCTCGGGGGGTGCGTGCGTGTGTG |
| | | TGTGCGTGGGGAGCGCCGCGTGCGGCCCGCGCTGCCCGGCGGCTGTGAGCG |
| | | CTGCGGGCGCGGCGCGGGGCTTTGTGCGCTCCGCGTGTGCGCGAGGGGAGC |
| | | GCGGCCGGGGGCGGTGCCCCGCGGTGCGGGGGGGCTGCGAGGGGAACAAAG |
| | | GCTGCGTGCGGGGTGTGTGCGTGGGGGGGTGAGCAGGGGGTGTGGGCGCGG |
| | | CGGTCGGGCTGTAACCCCCCCCTGCACCCCCCTCCCCGAGTTGCTGAGCAC |
| | | GGCCCGGCTTCGGGTGCGGGGCTCCGTGCGGGGCGTGGCGCGGGGCTCGCC |
| | | GTGCCGGGCGGGGGGTGGCGGCAGGTGGGGGTGCCGGGCGGGGCGGGGCCG |
| | | CCTCGGGCCGGGGAGGGCTCGGGGGAGGGGCGCGGCGGCCCCGGAGCGCCG |
| | | GCGGCTGTCGAGGCGCGGCGAGCCGCAGCCATTGCCTTTTATGGTAATCGT |
| | | GCGAGAGGGCGCAGGGACTTCCTTTGTCCCAAATCTGGCGGAGCCGAAATC |
| | | TGGGAGGCGCCGCCGCACCCCCTCTAGCGGGCGCGGGCGAAGCGGTGCGGC |
| | | GCCGGCAGGAAGGAAATGGGCGGGGAGGGCCTTCGTGCGTCGCCGCGCCGC |
| | | CGTCCCCTTCTCCATCTCCAGCCTCGGGGCTGCCGCAGGGGGACGGCTGCC |
| | | TTCGGGGGGGACGGGGCAGGGCGGGGTTCGGCTTCTGGCGTGTGACCGGCG |
| | | GCTCTAGAGCCTCTGCTAACCATGTTCATGCCTTCTTCTTTTTCCTACAGC |
| | | TCCTGGGCAACGTGCTGGTTGTTGTGCTGTCTCATCATTTTGGCAAAGAAT |
| | | TCgCTAGTGGATCcaGGCCGCCTGGGCCCGTTAACACCATGTTCGTATTCC |
| | | TTGTCCTCCTCCCCCTCGTCAGCTCCCAGTGTGTCAATCTGACAACTCGAA |
| | | CTCAGCTCCCACCCGCATACACCAATAGTTTTACCCGAGGAGTCTATTATC |
| | | CCGACAAAGTTTTCAGgTCTAGTGTGTTGCATAGCACGCAGGACCTGTTCT |
| | | TGCCGTTTTTTTCAAATGTGACATGGTTCCATGCCATCCATGTCTCTGGTA |
| | | CGAACGGGACCAAAAGGTTTGATAACCCCGTTCTTCCATTTAACGACGGGG |
| | | TTTATTTCGCCTCTACTGAAAAGTCTAATATAATACGAGGTTGGATTTTTG |
| | | GGACAACACTGGACTCTAAAACGCAATCTCTCCTCATAGTAAATAATGCGA |
| | | CGAATGTCGTAATCAAAGTATGTGAGTTTCAGTTTTGCAACGACCCTTTTC |
| | | TTGGTGTATACTATCACAAGAACAACAAGTCTTGGATGGAAAGTGAATTtA |
| | | GAGTCTACAGCTCCGCAAACAACTGTACCTTTGAGTACGTCAGTCAGCCCT |
| | | TCCTTATGGACCTCGAAGGCAAACAGGGGAACTTCAAGAATCTGCGCGAGT |
| | | TCGTGTTCAAGAATATCGACGGCTATTTCAAGATATATTCAAAACATACAC |
| | | CAATCAATTTGGTACGCGATTTGCCGCAGGGCTTCTCTGCTCTTGAGCCTC |
| | | TCGTAGATTTGCCTATAGGGATCAATATTACTCGCTTTCAAACTCTCCTTG |
| | | CCCTTCATCGGTCCTACCTGACACCAGGCGACAGCTCCAGTGGGTGGACAG |
| | | CTGGTGCAGCGGCTTACTACGTCGGCTACCTCCAACCCAGGACTTTTTTGC |
| | | TGAAATATAATGAGAATGGTACGATTACTGACGCCGTCGATTGTGCCCTCG |
| | | ACCCATTGAGCGAAACTAAATGCACCCTGAAAAGCTTTACGGTTGAGAAAG |
| | | GGATATATCAGACATCTAATTTTCGCGTTCAACCCACGGAATCCATTGTAC |
| | | GATTTCCCAACATTACCAATTTGTGTCCTTTTGGTGAGGTATTTAATGCAA |
| | | CCAGATTCGCGAGTGTGTATGCCTGGAATAGGAAAAGAATTTCTAACTGTG |
| | | TAGCAGACTACAGCGTTCTGTATAATAGTGCGTCCTTTAGTACGTTCAAGT |
| | | GCTACGGAGTAAGCCCGACAAAGTTGAACGATTTGTGTTTCACTAACGTCT |
| | | ATGCCGATAGCTTCGTCATCAGGGGAGACGAAGTAAGGCAAATTGCACCAG |
| | | GCCAGACCGGaAAAATAGCCGACTACAACTATAAGTTGCCGGACGATTTCA |
| | | CCGGGTGTGTCATCGCGTGGAATAGTAATAACCTGGACTCCAAGGTGGGCG |
| | | GCAACTACAACTACCTGTACCGATTGTTCCGCAAGTCTAACCTCAAGCCCT |
| | | TTGAACGAGACATATCCACCGAGATATACCAAGCGGGAAGCACTCCCTGTA |
| | | ATGGCGTTGAAGGTTTCAACTGCTATTTTCCTCTCCAGTCCTATGGCTTCC |
| | | AACCTACCAATGGAGTAGGCTATCAACCGTATCGAGTCGTGGTGCTGAGTT |
| | | TCGAGCTCCTGCACGCTCCAGCGACCGTTTGTGGTCCTAAGAAAAGTACAA |
| | | ACCTCGTAAAAAATAAGTGCGTAAACTTTAACTTTAACGGTCTCACAGGAA |
| | | CGGGAGTTCTTACAGAGTCCAATAAGAAGTTCCTGCCCTTTCAGCAGTTTG |
| | | GGCGCGACATCGCGGACACCACAGATGCAGTCAGGGACCCGCAGACTCTTG |
| | | AGATCCTGGAcATCACCCCATGCTCTTTTGGCGGTGTGAGTGTCATTACAC |
| | | CCGGGACCAACACCAGCAATCAGGTTGCTGTGCTCTATCAAGACGTCAACT |
| | | GTACGGAAGTCCCAGTTGCAATACACGCGGACCAGTTGACGCCGACGTGGC |
| | | GGGTTTATTCTACCGGCTCAAACGTCTTTCAAACTAGAGCCGGTTGTTTGA |
| | | TCGGGGCGGAGCATGTGAACAATTCATACGAATGTGATATTCCCATAGGAG |
| | | CCGGAATATGTGCTTCTTACCAGACCCAAACCAACTCTCCGAGACGAGCCC |
| | | GGTCCGTAGCCAGTCAAAGCATAATTGCGTACACCATGAGCCTCGGTGCAG |
| | | AAAACTCAGTTGCGTACTCAAATAACTCCATTGCCATCCCAACAAATTTTA |
| | | CCATATCAGTGACCACTGAAATCCTTCCAGTGAGCATGACTAAAACAAGTG |
| | | TAGATTGCACAATGTAtATTTGCGGAGACTCAACTGAGTGCTCTAACCTCC |
| | | TGTTGCAGTACGGTTCATTCTGTACCCAGCTCAATCGCGCACTTACGGGAA |
| | | TAGCAGTAGAACAAGATAAGAATACTCAGGAAGTCTTCGCTCAAGTAAAGC |
| | | AAATCTACAAGACGCCCCCCATTAAGGACTTTGGGGGTTTTAATTTTAGCC |
| | | AAATACTCCCGGAcCCCAGTAAACCCTCTAAGAGGTCATTTATAGAAGACC |
| | | TGTTGTTTAATAAAGTTACTCTGGCCGATGCTGGCTTTATTAAACAATACG |
| | | GTGATTGTTTGGGCGACATCGCGGCGCGGGATCTGATATGCGCCCAAAAAT |
| | | TTAACGGTCTCACTGTGTTGCCGCCTTTGCTCACAGACGAGATGATAGCTC |
| | | AGTACACATCAGCGTTGTTGGCGGGTACCATAACGTCtGGATGGACATTCG |
| | | GAGCAGGGGCGGCCTTGCAAATACCATTCGCAATGCAAATGGCATACAGGT |
| | | TCAACGGGATAGGTGTTACACAGAATGTACTTTACGAGAACCAGAAATTGA |
| | | TTGCAAACCAGTTTAACTCTGCGATTGGCAAAATTCAGGATAGCCTCAGCT |
| | | CCACCGCGTCTGCACTGGGAAAACTCCAAGATGTTGTgAACCAAAATGCCC |
| | | AAGCCCTCAATACACTTGTGAAGCAACTCTCCAGCAATTTCGGTGCAATAA |
| | | GTAGTGTGCTGAACGACATACTTTCTCGGTTGGACAAAGTAGAGGCGGAGG |
| | | TGCAAATCGACAGACTCATAACAGGTCGACTGCAATCATTGCAAACGTATG |
| | | TTACTCAGCAATTGATACGGGCGGCGGAGATTCGAGCGTCTGCCAACCTCG |
| | | CCGCGACTAAAATGTCTGAGTGTGTGCTCGGTCAGTCAAAACGCGTGGACT |
| | | TCTGTGGAAAGGGATACCATCTCATGTCCTTCCCTCAGTCTGCCCCTCACG |
| | | GAGTAGTCTTTCTTCAtGTGACCTATGTTCCAGCACAGGAGAAAAATTTTA |
| | | CCACAGCGCCCGCGATATGCCACGATGGCAAAGCTCATTTTCCGCGAGAGG |
| | | GAGTATTTGTCTCTAACGGCACCCATTGGTTTGTAACCCAGCGCAATTTTT |
| | | ACGAACCTCAGATAATCACCACTGACAACACCTTTGTTAGTGGTAATTGTG |
| | | ATGTAGTGATCGGGATAGTgAACAATACAGTGTACGACCCCCTGCAACCTG |
| | | AGCTGGACTCCTTTAAAGAAGAACTCGACAAGTACTTCAAAAACCACACTT |
| | | CCCCGGATGTCGATTTGGGTGATATATCTGGCATCAATGCGAGTGTAGTCA |
| | | ACATACAGAAGGAAATCGACAGGCTCAATGAAGTAGCAAAAAACCTGAATG |
| | | AATCCCTCATAGAcCTCCAAGAGCTGGGCAAATATGAGCAGTACATTAAAT |
| | | GGCCCTGGTACATTTGGCTGGGCTTCATCGCTGGCCTGATAGCGATAGTGA |
| | | TGGTTACTATTATGCTCTGCTGTATGACGAGTTGTTGCAGCTGCCTCAAAG |
| | | GGTGTTGTTCTTGCGGcTCCTGTTGTAAGTTCGACGAGGATGATTCAGAGC |
| | | CCGTTCTTAAGGGTGTAAAACTCCATTACACTGGAGGAGGAGGTTACCCAT |
| | | ACGATGTTCCAGATTACGCTTAGTAAACTAGTAACGTCTCGAGTTTAAACC |
| | | TGGAAAAACATGGAGCAATCACAAGTAGCAACACAGCAGCTACCAATGCTG |
| | | CTTGTGCCTGGCTAGAAGCACAAGAGGAGGAGGAGGTGGGTTTTCCAGTCA |
| | | CACCTCAGACAATCAACCTCTGGATTACAAAATTTGTGAAAGATTGACTGG |
| | | TATTCTTAACTATGTTGCTCCTTTTACGCTATGTGGATACGCTGCTTTAAT |
| | | GCCTTTGTATCATGCTATTGCTTCCCGTATGGCTTTCATTTTCTCCTCCTT |
| | | GTATAAATCCTGGTTGCTGTCTCTTTATGAGGAGTTGTGGCCCGTTGTCAG |
| | | GCAACGTGGCGTGGTGTGCACTGTGTTTGCTGACGCAACCCCCACTGGTTG |
| | | GGGCATTGCCACCACCTGTCAGCTCCTTTCCGGGACTTTCGCTTTCCCCCT |
| | | CCCTATTGCCACGGCGGAACTCATCGCCGCCTGCCTTGCCCGCTGCTGGAC |
| | | AGGGGCTCGGCTGTTGGGCACTGACAATTCCGTGGTGTTGTCGGGGAAGCT |
| | | GACGTCCTTTCCATGGCTGCTCGCCTGTGTTGCCACCTGGATTCTGCGCGG |
| | | GACGTCCTTCTGCTACGTCCCTTCGGCCCTCAATCCAGCGGACCTTCCTTC |
| | | CCGCGGCCTGCTGCCGGCTCTGCGGCCTCTTCCGCGTCTTCGCCTTCGCCC |
| | | TCAGACGAGTCGGATCTCCCTTTGGGCCGCCTCCCCGCCTGGAATTCGAGC |
| | | TCGGTACCTTTAAGACCAATGACTTACAAGGCAGCTGTAGATCTTAGCCAC |
| | | TTTTTAAAAGAAAAGGGGGGACTGGAAGGGCTAATTCACTCCCAAAGAAGA |
| | | CAAGATATCCTTGATCTGTGGATCTACCACACACAAGGCTACTTCCCTGAT |
| | | TGACAGAACTACACACCAGGGCCAGGGGTCAGATATCCACTGACCTTTGGA |
| | | TGGTGCTACAAGCTAGTACCAGTTGAGCCAGATAAGATAGAAGAGGCCAAT |
| | | AAAGGAGAGAACACCAGCTTGTTACACCCTGTGAGCCTGCATGGGATGGAT |
| | | GACCCGGAGAGAGAAGTGTTAGAGTGGAGGTTTGACAGCCGCCTAGCATTT |
| | | CATCACGTGGCCCGAGAGCTGCATCCGGACTGTACTGGGTCTCTCTGGTTA |
| | | GACCAGATCTGAGCCTGGGAGCTCTCTGGCTAACTAGGGAACCCACTGCTT |
| | | AAGCCTCAATAAAGCTTGCCTTGAGTGCTTCAAGTAGTGTGTGCCCGTCTG |
| | | TTGTGTGACTCTGGTAACTAGAGATCCCTCAGACCCTTTTAGTCAGTGTGG |
| | | AAAATCTCTAGCAGGGCCGCTTTAAACCCGCTGATCAGCCTCGACTGTGCC |
| | | TTCTAGTTGCCAGCCATCTGTTGTTTGCCCCTCCCCCGTGCCTTCCTTGAC |
| | | CCTGGAAGGTGCCACTCCCACTGTCCTTTCCTAATAAAATGAGGAAATTGC |
| | | ATCGCATTGTCTGAGTAGGTGTCATTCTATTCTGGGGGGTGGGGTGGGGCA |
| | | GGACAGCAAGGGGGAGGATTGGGAAGACAATAGCAGGCATGCTGGGGATGC |
| | | GGTGGGCTCTATGGCTTCTGAGGCGGAAAGAACCAGCTGGGGCTCTAGGGG |
| | | GTATCCCCACGCGCCCTGTAGCGGCGCATTAAGCGCGGCGGGTGTGGTGGT |
| | | TACGCGCAGCGTGACCGCTACACTTGCCAGCGCCCTAGCGCCCGCTCCTTT |
| | | CGCTTTCTTCCCTTCCTTTCTCGCCACGTTCGCCGGCTTTCCCCGTCAAGC |
| | | TCTAAATCGGGGCATCCCTTTAGGGTTCCGATTTAGTGCTTTACGGCACCT |
| | | CGACCCCAAAAAACTTGATTAGGGTGATGGTTCACGTAGTGGGCCATCGCC |
| | | CTGATAGACGGTTTTTCGCCCTTTGACGTTGGAGTCCACGTTCTTTAATAG |
| | | TGGACTCTTGTTCCAAACTGGAACAACACTCAACCCTATCTCGGTCTATTC |
| | | TTTTGATTTATAAGGGATTTTGGGGATTTCGGCCTATTGGTTAAAAAATGA |
| | | GCTGATTTAACAAAAATTTAACGCGAATTAATTCTGTGGAATGTGTGTCAG |
| | | TTAGGGTGTGGAAAGTCCCCAGGCTCCCCAGGCAGGCAGAAGTATGCAAAG |
| | | CATGCATCTCAATTAGTCAGCAACCAGGTGTGGAAAGTCCCCAGGCTCCCC |
| | | AGCAGGCAGAAGTATGCAAAGCATGCATCTCAATTAGTCAGCAACCATAGT |
| | | CCCGCCCCTAACTCCGCCCATCCCGCCCCTAACTCCGCCCAGTTCCGCCCA |
| | | TTCTCCGCCCCATGGCTGACTAATTTTTTTTATTTATGCAGAGGCCGAGGC |
| | | CGCCTCTGCCTCTGAGCTATTCCAGAAGTAGTGAGGAGGCTTTTTTGGAGG |
| | | CCTAGGCTTTTGCAAAAAGCTCCCGGGAGCTTGTATATCCATTTTCGGATC |
| | | TGATCAGCACGTGTTGACAATTAATCATCGGCATAGTATATCGGCATAGTA |
| | | TAATACGACAAGGTGAGGAACTAAACCATGGCCAAGTTGACCAGTGCCGTT |
| | | CCGGTGCTCACCGCGCGCGACGTCGCCGGAGCGGTCGAGTTCTGGACCGAC |
| | | CGGCTCGGGTTCTCCCGGGACTTCGTGGAGGACGACTTCGCCGGTGTGGTC |
| | | CGGGACGACGTGACCCTGTTCATCAGCGCGGTCCAGGACCAGGTGGTGCCG |
| | | GACAACACCCTGGCCTGGGTGTGGGTGCGCGGCCTGGACGAGCTGTACGCC |
| | | GAGTGGTCGGAGGTCGTGTCCACGAACTTCCGGGACGCCTCCGGGCCGGCC |
| | | ATGACCGAGATCGGCGAGCAGCCGTGGGGGCGGGAGTTCGCCCTGCGCGAC |
| | | CCGGCCGGCAACTGCGTGCACTTCGTGGCCGAGGAGCAGGACTGACACGTG |
| | | CTACGAGATTTCGATTCCACCGCCGCCTTCTATGAAAGGTTGGGCTTCGGA |
| | | ATCGTTTTCCGGGACGCCGGCTGGATGATCCTCCAGCGCGGGGATCTCATG |
| | | CTGGAGTTCTTCGCCCACCCCAACTTGTTTATTGCAGCTTATAATGGTTAC |
| | | AAATAAAGCAATAGCATCACAAATTTCACAAATAAAGCATTTTTTTCACTG |
| | | CATTCTAGTTGTGGTTTGTCCAAACTCATCAATGTATCTTATCATGTCTGT |
| | | ATACCGTCGACCTCTAGCTAGAGCTTGGCGTAATCATGGTCATAGCTGTTT |
| | | CCTGTGTGAAATTGTTATCCGCTCACAATTCCACACAACATACGAGCCGGA |
| | | AGCATAAAGTGTAAAGCCTGGGGTGCCTAATGAGTGAGCTAACTCACATTA |
| | | ATTGCGTTGCGCTCACTGCCCGCTTTCCAGTCGGGAAACCTGTCGTGCCAG |
| | | CTGCATTAATGAATCGGCCAACGCGCGGGGAGAGGCGGTTTGCGTATTGGG |
| | | CGCTCTTCCGCTTCCTCGCTCACTGACTCGCTGCGCTCGGTCGTTCGGCTG |
| | | CGGCGAGCGGTATCAGCTCACTCAAAGGCGGTAATACGGTTATCCACAGAA |
| | | TCAGGGGATAACGCAGGAAAGAACATGTGAGCAAAAGGCCAGCAAAAGGCC |
| | | AGGAACCGTAAAAAGGCCGCGTTGCTGGCGTTTTTCCATAGGCTCCGCCCC |
| | | CCTGACGAGCATCACAAAAATCGACGCTCAAGTCAGAGGTGGCGAAACCCG |
| | | ACAGGACTATAAAGATACCAGGCGTTTCCCCCTGGAAGCTCCCTCGTGCGC |
| | | TCTCCTGTTCCGACCCTGCCGCTTACCGGATACCTGTCCGCCTTTCTCCCT |
| | | TCGGGAAGCGTGGCGCTTTCTCAATGCTCACGCTGTAGGTATCTCAGTTCG |
| | | GTGTAGGTCGTTCGCTCCAAGCTGGGCTGTGTGCACGAACCCCCCGTTCAG |
| | | CCCGACCGCTGCGCCTTATCCGGTAACTATCGTCTTGAGTCCAACCCGGTA |
| | | AGACACGACTTATCGCCACTGGCAGCAGCCACTGGTAACAGGATTAGCAGA |
| | | GCGAGGTATGTAGGCGGTGCTACAGAGTTCTTGAAGTGGTGGCCTAACTAC |
| | | GGCTACACTAGAAGGACAGTATTTGGTATCTGCGCTCTGCTGAAGCCAGTT |
| | | ACCTTCGGAAAAAGAGTTGGTAGCTCTTGATCCGGCAAACAAACCACCGCT |
| | | GGTAGCGGTGGTTTTTTTGTTTGCAAGCAGCAGATTACGCGCAGAAAAAAA |
| | | GGATCTCAAGAAGATCCTTTGATCTTTTCTACGGGGTCTGACGCTCAGTGG |
| | | AACGAAAACTCACGTTAAGGGATTTTGGTCATGAGATTATCAAAAAGGATC |
| | | TTCACCTAGATCCTTTTAAATTAAAAATGAAGTTTTAAATCAATCTAAAGT |
| | | ATATATGAGTAAACTTGGTCTGACAGTTACCAATGCTTAATCAGTGAGGCA |
| | | CCTATCTCAGCGATCTGTCTATTTCGTTCATCCATAGTTGCCTGACTCCCC |
| | | GTCGTGTAGATAACTACGATACGGGAGGGCTTACCATCTGGCCCCAGTGCT |
| | | GCAATGATACCGCGAGACCCACGCTCACCGGCTCCAGATTTATCAGCAATA |
| | | AACCAGCCAGCCGGAAGGGCCGAGCGCAGAAGTGGTCCTGCAACTTTATCC |
| | | GCCTCCATCCAGTCTATTAATTGTTGCCGGGAAGCTAGAGTAAGTAGTTCG |
| | | CCAGTTAATAGTTTGCGCAACGTTGTTGCCATTGCTACAGGCATCGTGGTG |
| | | TCACGCTCGTCGTTTGGTATGGCTTCATTCAGCTCCGGTTCCCAACGATCA |
| | | AGGCGAGTTACATGATCCCCCATGTTGTGCAAAAAAGCGGTTAGCTCCTTC |
| | | GGTCCTCCGATCGTTGTCAGAAGTAAGTTGGCCGCAGTGTTATCACTCATG |
| | | GTTATGGCAGCACTGCATAATTCTCTTACTGTCATGCCATCCGTAAGATGC |
| | | TTTTCTGTGACTGGTGAGTACTCAACCAAGTCATTCTGAGAATAGTGTATG |
| | | CGGCGACCGAGTTGCTCTTGCCCGGCGTCAATACGGGATAATACCGCGCCA |
| | | CATAGCAGAACTTTAAAAGTGCTCATCATTGGAAAACGTTCTTCGGGGCGA |
| | | AAACTCTCAAGGATCTTACCGCTGTTGAGATCCAGTTCGATGTAACCCACT |
| | | CGTGCACCCAACTGATCTTCAGCATCTTTTACTTTCACCAGCGTTTCTGGG |
| | | TGAGCAAAAACAGGAAGGCAAAATGCCGCAAAAAAGGGAATAAGGGCGACA |
| | | CGGAAATGTTGAATACTCATACTCTTCCTTTTTCAATATTATTGAAGCATT |
| | | TATCAGGGTTATTGTCTCATGAGCGGATACATATTTGAATGTATTTAGAAA |
| | | AATAAACAAATAGGGGTTCCGCGCACATTTCCCCGAAAAGTGCCACCTGAC |
| | | GTC |

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Construction of a Lentiviral Vector Encoding a Codon-Optimized SARS-CoV-2 Spike Protein

To create a multi-purpose vector for the expression of SARS-CoV-2 Spike protein, a lentiviral vector with a SARS-CoV-2 Spike protein open reading frame, driven by a CAG promoter, was constructed. The resulting pBOB-CAG vector of the present invention is significantly more efficient than the standard lentiviral vector and underwent multiple iterations of small changes that led to many small improvements that are multiplicative. These small improvements in aggregate led to about a cumulative 100× improvement in expression. This improvement was achieved through manipulations of viral infection, reverse transcription, expression as well as production level optimization that make improvement of expression the final measure of biological relevance about two orders of magnitude better than common commercial sources of lentiviral vectors. Improvements included keeping the CMV promoter in 293T cells, which maximized expression. The distance of the CMV promoter to the transcriptional initiation position was also optimized. The remnants of the U3 in the long terminal repeat (LTR) was also optimized. A splice acceptor was introduced upstream of the cPPT. In addition, the cPPT spacing was optimized. The CAG promoter was demonstrated to achieve the most consistent expression across a large variety of tissues in mice as well as human hematopoietic derived cells. The spacing of the multiple cloning site to the promoter was optimized for use with the BamHI cloning site. The Woodchuck Hepatitis Virus (WHV) Post-transcriptional Regulatory Element (WPRE) sequence contains a small deletion at its 5′ side to increase RNA stability about 20%. A number of U3 deletion variants were tested and a slightly smaller deletion was selected that increased titer as well as expression. A genomic region from the human genome from the original integration site was removed. This flanking sequence was a source of plasmid instability and decreased plasmid production as well as stability in the production of the lentiviral vector. Usage of the plasmid in transfection using 2×BES-buffered saline (BBS) instead of more conventional HEPES-buffered saline (HBS) and other lipid-based transfection reagents increased titer at least 10×. Overall, the changes optimized the production, titer and expression of transgenes to result commonly in about two orders of magnitude better than common commercial sources of lentiviral vectors.

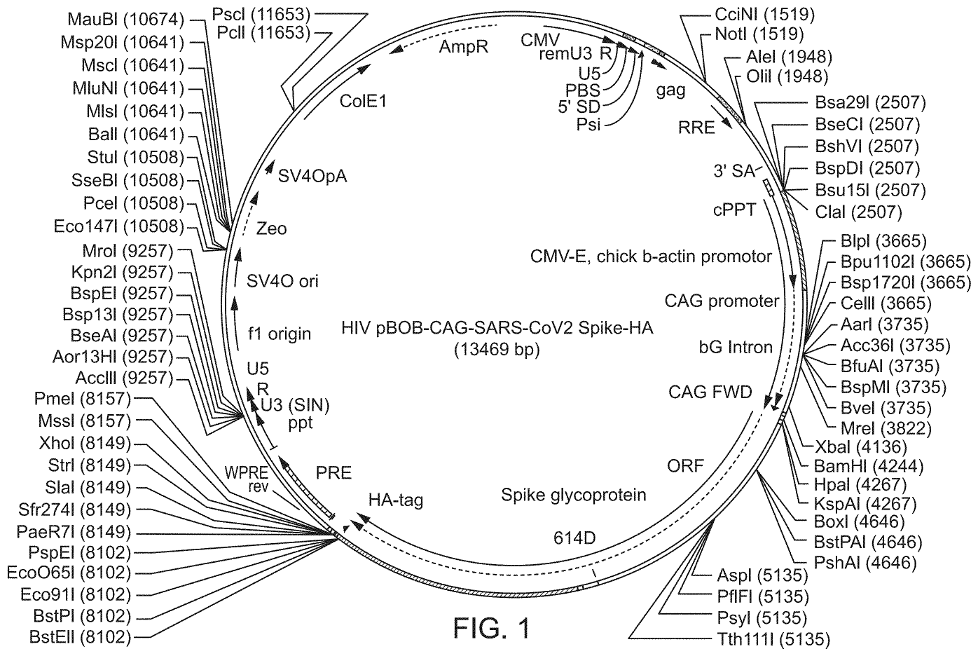

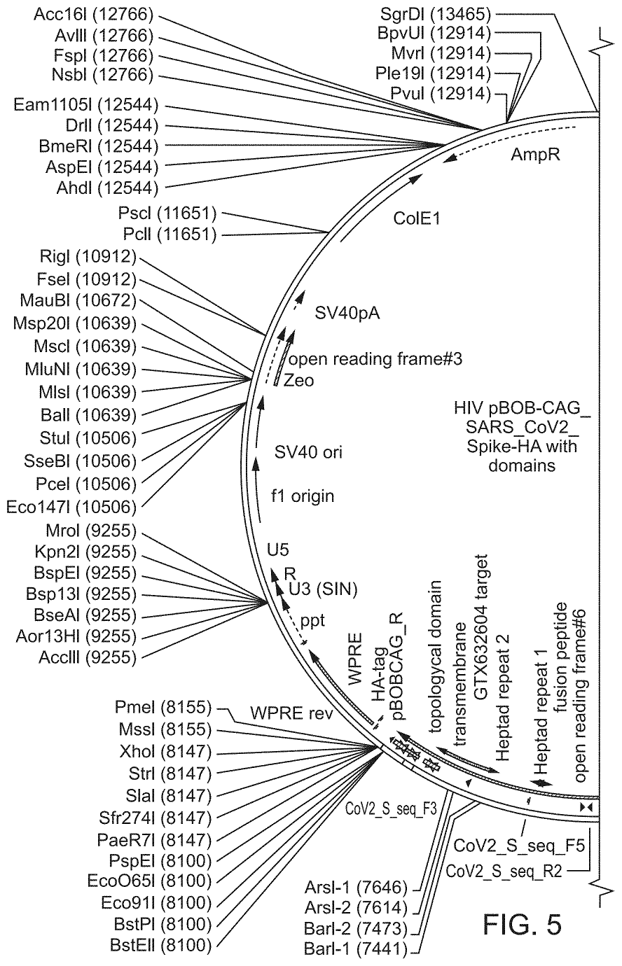

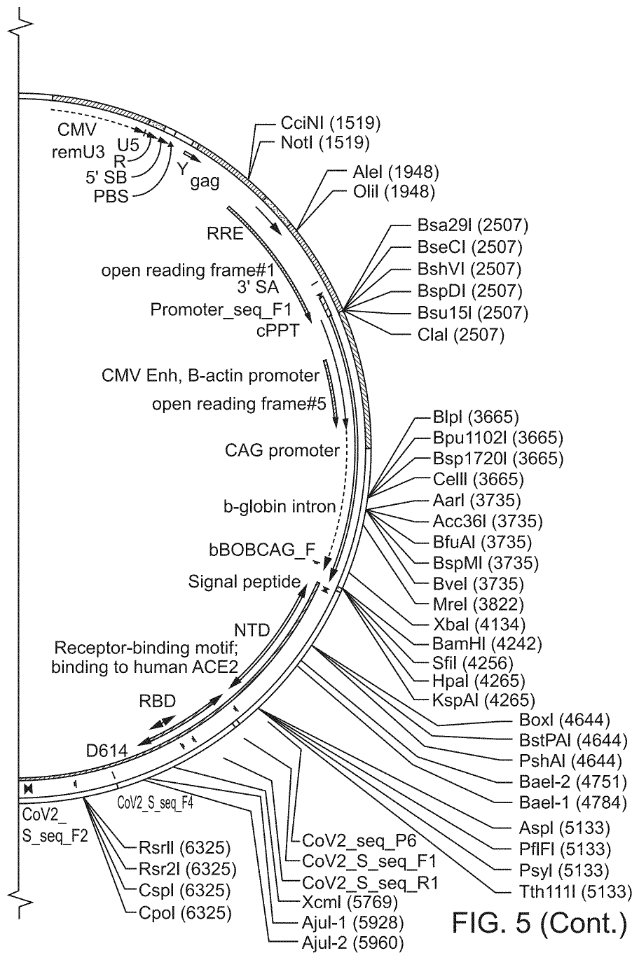

The lentiviral vector, with the design shown in FIG. 1 or FIG. 5, was reprogrammed as detailed above from a self-inactivating pBOB lentiviral vector for gene expression of an HA-tagged CoV-2 SARS Spike Protein by inserting an ORF encoding a codon optimized SARS CoV-2 Spike Protein (e.g., SEQ ID NO: 7; SEQ ID NO: 9) into the BamHI restriction site. A CAG promoter was inserted into the Bsa291 restriction site upstream of the ORF.

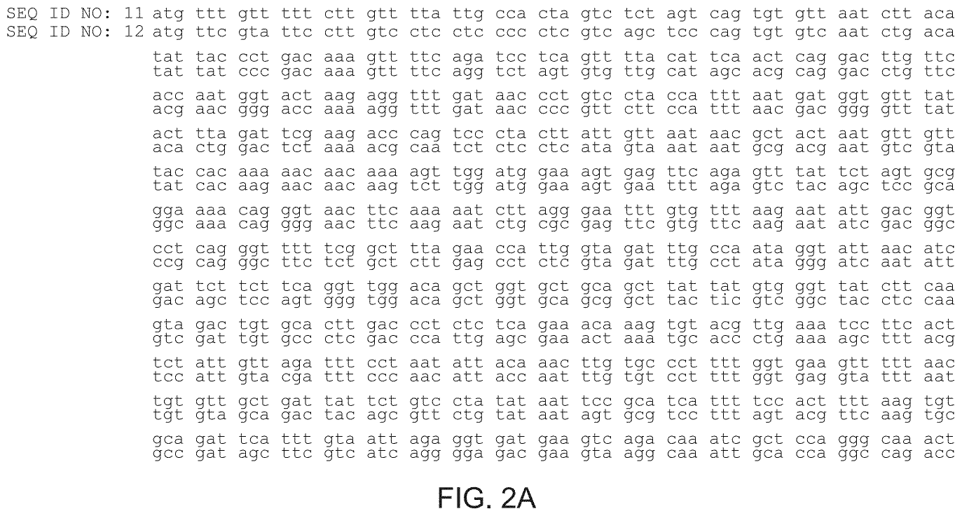

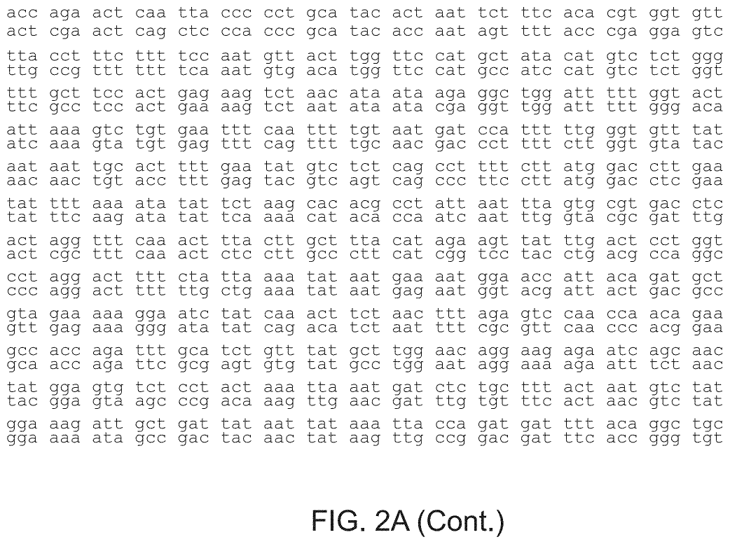

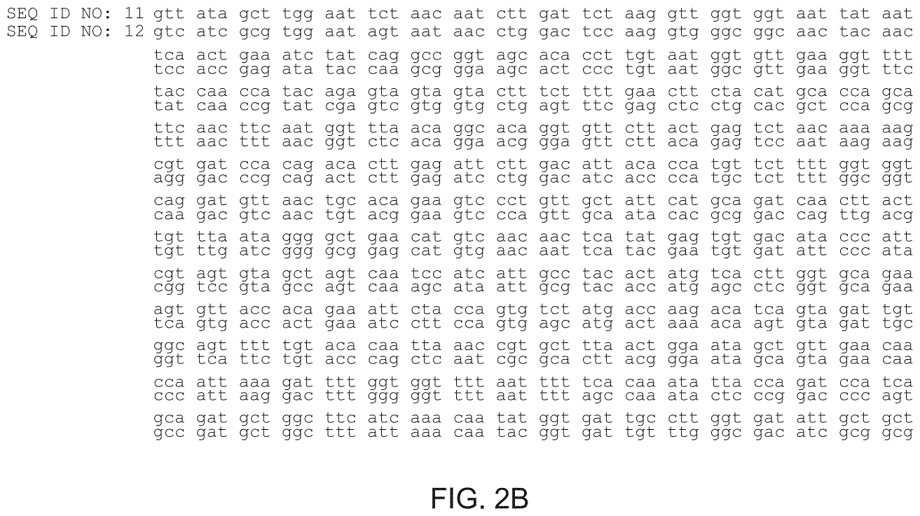

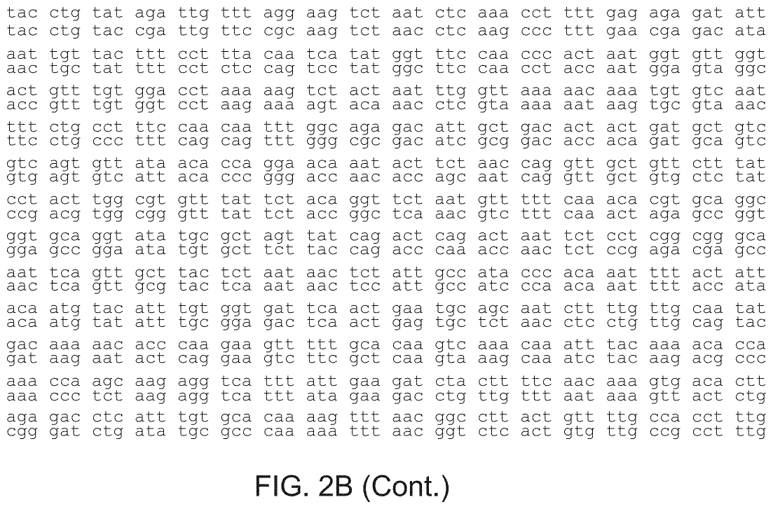

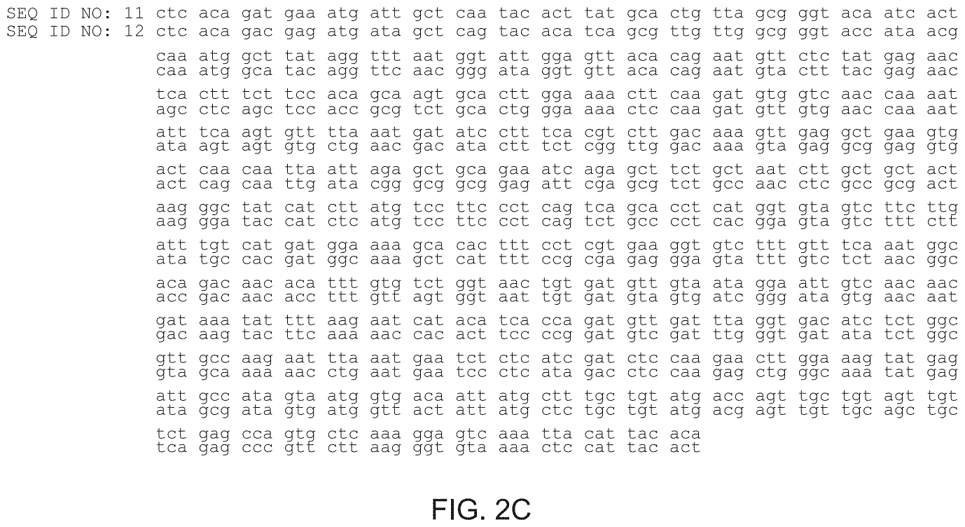

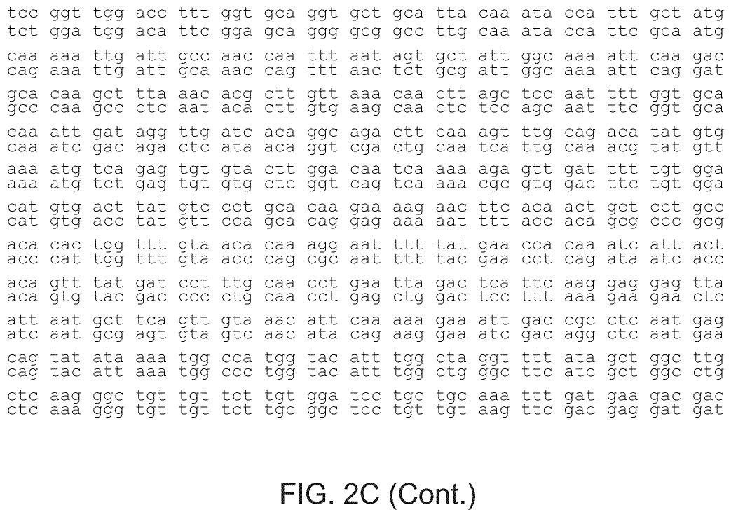

The open reading frame sequence containing a codon optimized sequence encoding the CoV-2 SARS Spike Protein with an N-terminal HA-TAG was generated using a codon optimization program amenable to mammalian expression. The wild-type CoV-2 SARS Spike Protein nucleic acid sequence (SEQ ID NO: 1) was subjected to synonymous mutations that did not alter the amino acid sequence and to simultaneously minimize the number of repeat sequences and codon-optimize for expression in mammalian cells and tissues. A comparison of the SEQ ID NO: 1 and SEQ ID NO: 3 open reading frames is shown in FIG. 2. HA-tag sequence was added to the terminal end of the sequence by conventional PCR primer methods and then subcloned into the pBOB vector for bacterial production, isolation, and purification.

### Example 2: System to Evaluate the Significance of SARS-CoV2 Variant Mutations

The SARS-CoV2 coronavirus responsible for the COVID19 pandemic has been reported to have a relatively low mutation rate. Nevertheless, a few prevalent variants have arisen that give the appearance of undergoing positive selection as they are becoming increasingly widespread over time. Most prominent among these is the D614G amino acid substitution in the SARS-CoV2 Spike protein, which mediates viral entry. The D614G substitution, however, is in linkage disequilibrium with the ORF1b P314L mutation where both mutations almost invariably co-occur, making functional inferences problematic. In addition, the possibility of repeated new introductions of the mutant strain does not allow one to distinguish between a founder effect and an intrinsic genetic property of the virus. Here, we synthesized and expressed the WT and D614G variant SARS-Cov2 Spike protein, and report that using a SARS-CoV2 Spike protein pseudotyped lentiviral vector we observe that the D614G variant Spike has >½ log₁₀ increased infectivity in human cells expressing the human ACE2 protein as the viral receptor. The increased binding/fusion activity of the D614G Spike protein was corroborated in a cell fusion assay using Spike and ACE2 proteins expressed in different cells. These results are consistent with the possibility that the Spike D614G mutant increases the infectivity of SARS-CoV2.

The SARS-CoV2 Coronavirus that initiated the current global pandemic, appeared in late 2019 in Hubei province, China (P. Zhou, et al., A pneumonia outbreak associated with a new coronavirus of probable bat origin. Nature 579, 270-273 (2020)), (F. Wu, et al., A new coronavirus associated with human respiratory disease in China. Nature 579, 265-269 (2020)). The epidemic experienced a fast growth early on in the city of Wuhan as its epicenter in late 2019 and early January 2020 and then declined in China as a whole by the second half of February 2020. By the time the epidemic reached Europe, a variant strain had appeared that carried a missense mutation in the Spike glycoprotein that substituted the aspartate at position 614 for a glycine in isolates identified in Germany, Italy and Mexico (P. Stefanelli, et al., Whole genome and phylogenetic analysis of two SARSCoV-2 strains isolated in Italy in January and February 2020: Additional clues on multiple introductions and further circulation in Europe. Eurosurveillance 25, 1-5 (2020)). This mutation is in linkage disequilibrium with the ORF1b gene P314L substitution. In almost all cases ORF1b P314L and Spike D614G variants co-occur. The Spike glycoprotein is a type I membrane protein and the largest surface protein of the SARS-CoV2 coronavirus. It mediates infection of target cells through binding to its cognate receptor angiotensin converting enzyme 2 (ACE2) and then initiating viral-host cell membrane fusion (M. Hoffmann, et al., SARS-CoV-2 Cell Entry Depends on ACE2 and TMPRSS2 and Is Blocked by a Clinically Proven Protease Inhibitor. Cell 181, 271-280.e8 (2020)). After the appearance of the Spike D614G variant in the latter course of the Chinese epidemic, over time in most examined local epidemics an enrichment of the 614G Spike protein variant over the original 614D variant has been observed, leading to the hypothesis that the Spike D614G mutation is positively selected (B. Korber, et al., Spike mutation pipeline reveals the emergence of a more transmissible form of SARS-CoV-2. bioRxiv 4, 2020.04.29.069054 (2020))(Supplemental movie, https://nextstrain.org/ncov/global?c=gt-S_614), (J. Hadfield, et al., NextStrain: Real-time tracking of pathogen evolution. Bioinformatics 34, 4121-4123 (2018)). The caveat, however, is that due to possible multiple introductions and reintroduction events a founder effect could also explain the observed viral strain dynamics. Here, we present evidence that the D614G Spike mutant displays a slightly increased infectivity (˜5×) in ACE2-expressing cells without a contribution of the ORF1b P314L mutation, when tested in pseudotyped lentiviral vectors. This result provides a plausible mechanism for the increased observed infectivity inferred from epidemiological observations and is consistent with the positive selection hypothesis of the D614G mutation.

Methods

Pseudotyped lentiviral vectors were prepared, titered and partially purified as described in Tiscornia et al. (G. Tiscornia, O. Singer, I. M. Verma, Production and purification of lentiviral vectors. Nat. Protoc. 1, 241-245 (2006)). For small scale production, calcium phosphate transfection was substituted for Lipofectamine LTX using the manufacturer's recommended conditions. The Spike protein gene sequence was synthesized based on the original US isolate SARS-CoV2 hCoV19_USA EPI_ISL_414366 (GISAID) with human codon optimization and the addition of a C-terminal HA tag (IDT), and cloned by Gibson assembly using the NEB kit into the lentiviral pBOB-CAG vector. The D614G substitution was introduced with a single gBlock spanning the AspI and RsrII sites and Gibson assembly (NEB). Pseudotyping plasmids were generated by deletion of the external CMV promoter and 5′ LTR leaving the internal CAG promoter by restriction digestion, and self-ligation.

A stable Spike expressing 293T cell line was generated by infection with pBOB-CAG-SARS-CoV2-Spike-HA lentiviral vector https://www.addgene.org/141347/ followed by single cell cloning and screening by cell extract immunoblotting of individual clones with HA tag antibody (CST #3724) Immunofluorescence was performed following the Cell Signaling Technology® protocol. Primary antibodies were GeneTex 632604 anti-Spike antibody (mouse monoclonal) and anti-calnexin (C5C9) rabbit mAb (CST #2679) were detected with 2nd anti-Mouse IgG Alexa 488 (Life tech Cat #A11029) and anti-rabbit IgG Dylight 633 (Thermo Fisher #35563) antibodies. Imaging was performed with Zeiss AiryScan 880 microscope. Human and mouse ACE2 expressing cell lines were generated by lentiviral vector (pLV-human-ACE2 IRES Puro or pLV-mouse-ACE2 IRES Puro, gifts from Dr. Sumit Chanda) transduction followed by puromycin selection. Statistical analyses were performed using Fisher's 2 tailed T-test on MS Excel.

Results

The Spike protein is the largest structural protein of the SARS-CoV2 virus (F. Wu, et al., A new coronavirus associated with human respiratory disease in China. Nature 579, 265-269 (2020)). This type I trimeric membrane protein mediates the viral entry into target cells through the binding of its primary receptor ACE2, and possibly also interaction with additional receptors or co-receptors (J. L. Daly, et al., Neuropilin-1 is a host factor for SARS-CoV-2 infection. bioRxiv, 2020.06.05.134114 (2020)). Activation of the Spike protein for receptor binding, requires proteolytic processing by serine proteases, such as furin, at the polybasic site during its secretion from the producer cells, or alternatively by the target cell plasma membrane TMPRSS2 protease or endosomal proteases such as cathepsins, into the S1 and S2 domains (M. Hoffmann, et al., SARS-CoV-2 Cell Entry Depends on ACE2 and TMPRSS2 and Is Blocked by a Clinically Proven Protease Inhibitor. Cell 181, 271-280.e8 (2020), S. Belouzard, V. C. Chu, G. R. Whittaker, Activation of the SARS coronavirus spike protein via sequential proteolytic cleavage at two distinct sites. Proc. Natl. Acad. Sci. U.S.A 106, 5871-5876 (2009)). Upon binding to the ACE2 receptor, the S1 domain is shed and the S2 domain is exposed. The S2 domain has to be further proteolytically activated to expose the fusion peptide, which initiates membrane fusion of the viral and host cell membranes to mediate viral entry into the cytoplasm (Id.). The D614G mutation on the Spike protein is located at the C-terminus of the S1 fragment and outside of the receptor-binding domain, and thus is unlikely to directly influence ACE2 binding.

Cell Biology of Spike-Mediated Fusion

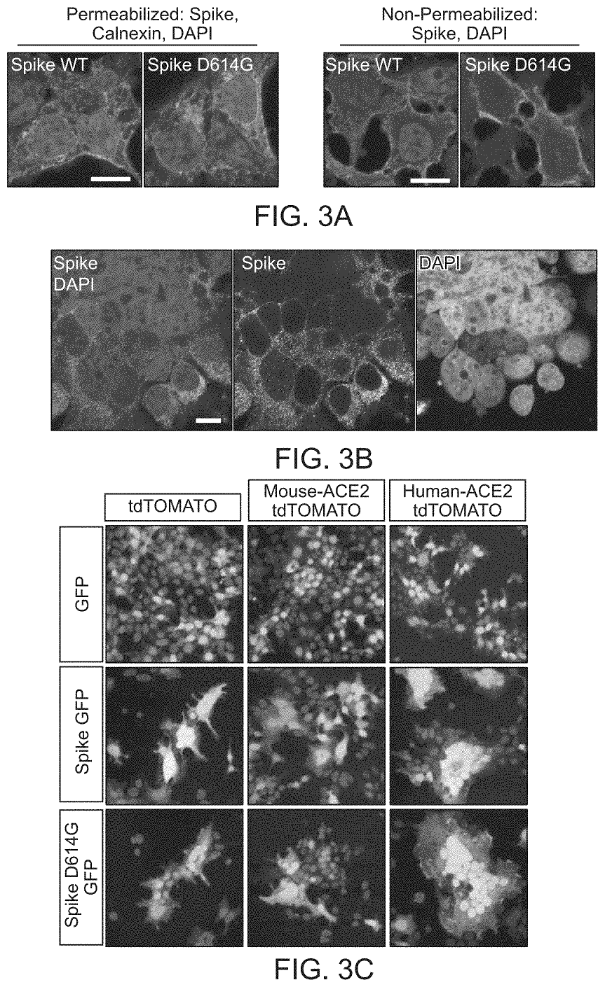

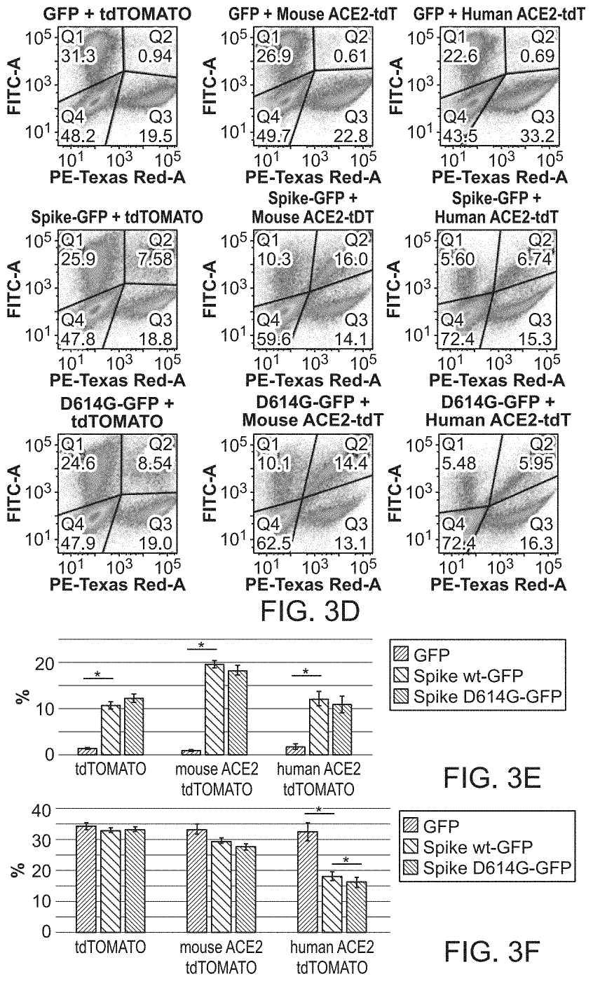

Expression of the Spike glycoprotein (GP) in 293T cells invariably led to some amount of cell fusion. This was observed in transient transfection (FIG. 3A) as well as in a stable cell line generated by infection with a lentiviral vector expressing Spike GP (FIG. 3B). The majority of expressed Spike GP was located in the endoplasmic reticulum (ER) as seen by calnexin colocalization in methanol-permeabilized cells (FIG. 3A). Spike was also observed on the nuclear membrane and to a lesser extent on the plasma membrane. Staining cells without permeabilization showed that Spike is readily expressed on the plasma membrane, in the absence of any other viral protein, and therefore should be capable of mediating cell fusion (FIG. 3A right panel). Significant localization differences between Spike WT and Spike D614G in transiently transfected cells was not observed. Higher levels of expression led to aberrant ER morphology probably due to ER-ER fusion events. In addition, we observed fusion of the outer nuclear membrane leading to syncytia with aggregated nuclei (FIG. 3B). To test the activity of the SARS Cov2 protein in vitro, we used a cell fusion assay in which 293T cells were either transfected with Spike or transfected with the Spike receptor ACE2 (S. Belouzard, V. C. Chu, G. R. Whittaker, Activation of the SARS coronavirus spike protein via sequential proteolytic cleavage at two distinct sites. Proc. Natl. Acad. Sci. U.S.A 106, 5871-5876 (2009)). In order to distinguish and visualize the Spike and ACE2-expressing cells, the Spike protein was co-transfected with EGFP and the ACE2 protein co-transfected with TdTomato. Cells were mixed, plated and then observed by immunofluorescence microscopy or Fluorescence Aided Cell Sorting (FACS) 24 hours after plating. The experiments were performed with both the original Spike 614D variant GP as well as the Spike 614G mutant GP, and mouse ACE2 and human ACE2 were compared. Mixing of both mouse and human ACE2-expressing cells with Spike GP expressing cells led to an increase in syncytia frequency as well as size, i.e. syncytia with more nuclei, as observed by microscopy (FIG. 3C). Both of these effects appeared to be further increased using human ACE2 compared to mouse ACE2. FACS analysis showed that transfection of Spike GP increased red/green double positive events indicative of fusion 7-8×. Inclusion of the mouse ACE2 in the TdTomato cells increased the fusions to ˜17-18× over the baseline without Spike (FIG. 3E). Use of the human ACE2 only increased measured fusion events 5-6× due to the loss of the large syncytia (FIG. 3E). In order to quantify the extent of fusion, we measured the depletion of syncytia rather than counting fused cells, as Spike-mediated fusion produced syncytia that were frequently large enough to be lost during passage through the FACS preparation mesh filters designed to eliminate cellular aggregates. Using this assay we observed an increase in fusion cell depletion when target cells were transfected with either mouse or human ACE2. Fusion efficiency was higher with human ACE2, but mouse ACE2 enhancement of fusion was clearly detectable by this assay, consistent with microscopy observations. The difference between Spike GP 614D and 614G with mouse ACE2 was not significant (p=0.0722). With the human ACE2 however, we observed an 11% enhancement of D614G GFP+ cell depletion compared to WT Spike, which although small in magnitude achieved statistical significance (p<0.02) (FIG. 3F).

Spike Pseudotyped Lentiviral Vectors

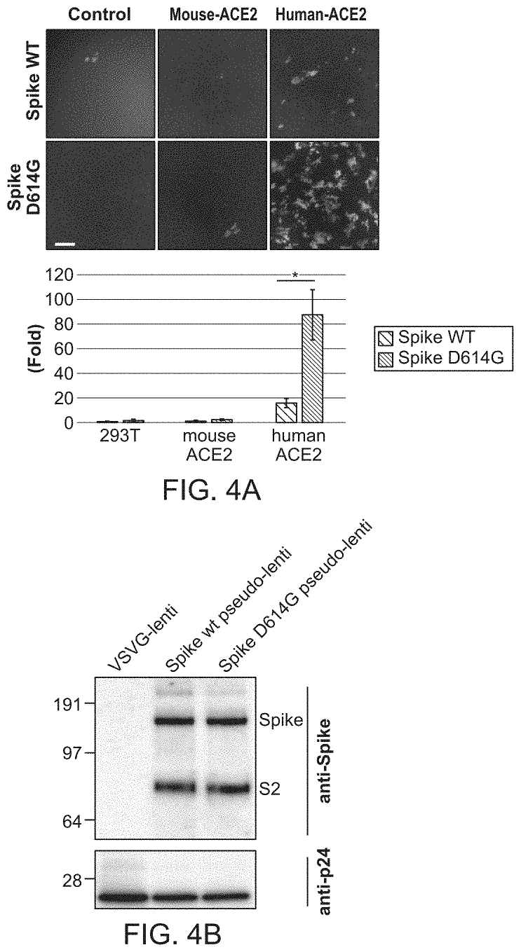

In order to obtain a better dynamic range assay, we generated SARS-CoV2 pseudotyped lentiviral vectors that incorporated Spike GP as their envelope protein carrying a payload expressing EGFP under the CAG promoter. This pseudotyped viral vector was used to infect mouse and human ACE2 expressing cells (X. Ou, et al., Characterization of spike glycoprotein of SARS-CoV-2 on virus entry and its immune cross-reactivity with SARS-CoV. Nat. Commun. 11 (2020)). The Spike pseudotyped lentiviral vector physical titer was normalized to HIV p24 Gag protein prior to infection for all viral vectors generated. Comparison of infection efficiencies of control 293T cells with stable cells expressing either mouse or human ACE2 demonstrated an increased infection efficiency (15×) in the human ACE2 expressing cells over the 293T cell background. A similar increase was not observed in mouse ACE2-expressing cells. Comparison of Spike 614G with Spike 614D showed a consistently enhanced infectivity of the Spike 614G variant (˜5×) (FIG. 4A) Co-transfection of TMPRSS2 in 293T cells did not enhance infectivity suggesting that proteolytic processing is not limiting in these cells (data not shown). Comparison of proteolytic processing of 614D and 614G Spike proteins by immunoblotting with an antibody against a juxtamembrane epitope on partially purified pseudotyped lentiviral vector virions showed neither enrichment of Spike incorporation nor proteolytic processing differences. This was observed both with partially purified viral particles as well as in viral supernatants (FIG. 4B).

Discussion

The emergence of the SARS-CoV2 Spike D614G and its rapid increase in prevalence has been striking (B. Korber, et al., Spike mutation pipeline reveals the emergence of a more transmissible form of SARS-CoV-2. bioRxiv 4, 2020.04.29.069054 (2020)). Observational population genetics alone could not resolve whether this mutation and/or the frequently co-occurring ORF1b P314L allele alter biological activity or are just an epiphenomenon underlying a founder effect due to repeated introductions. Here, in the present report we find functional evidence that the Spike D614G mutant has modestly different biochemical properties consistent with positive selection of the D614G mutant SARS-CoV2 virus variant. The increase in cell entry activity of pseudotyped lentiviral vectors and cell fusion allowed us to dissect the Spike protein function in isolation and test the activity of the D614G point mutation. In our work we observed no increases in expression, increased stoichiometry of Spike incorporation into pseudotyped viral vector particles, nor proteolytic processing that could account for the increased infectivity. In contrast, other reports have respectively reported increases and decreases in S2 proteolytic processing (L. Zhang, et al., The D614G mutation in the SARS-CoV-2 spike protein reduces 51 shedding and increases infectivity https://doi.org/10.1101/2020.06.12.148726; Z. Daniloski, X. Guo, N. E. Sanjana, The D614G mutation in SARS-CoV-2 Spike increases transduction of multiple human cell types https://doi.org/10.1101/2020.06.14.151357; J. Hu, et at, The D614G mutation of SARS-CoV-2 spike protein enhances viral infectivity 1 and decreases neutralization sensitivity to individual convalescent sera 2 Running Title: D614G mutant spike increases SARS-CoV-2 infectivity https://doi.org/10.1101/2020.06.20.161323; S. Ozono, et al., Naturally mutated spike proteins of SARS-CoV-2 variants show differential levels of cell entry. bioRxiv, 2020.06.15.151779 (2020). https://doi.org/10.1101/2020.06.15.151779; S. Isabel, et al., Evolutionary and structural analyses of SARS-CoV-2 D614G 3 spike protein mutation now documented worldwide https://doi.org/10.1101/2020.06.08.140459). Given the location of the 614 residue within the 51 fragment distal to the RBD, it is also unlikely that the D614G mutation directly affects the ACE2 binding activity. Also unlikely is an Effect on the S2′ proteolytic processing that activates the fusion peptide, as the 51 fragment with the 614 site is already released before S2′ proteolytic processing can occur. Not yet investigated are the post-binding steps leading to the release of 51 before the S2′ activation of the fusion peptide. Thus, a biochemical basis for increased infectivity is observed in the SARS-CoV2 D614G mutant consistent with positive selection observed in the field. Nevertheless, we cannot currently explain the basis for this increased infectivity, and therefore further work is needed to clarify the mechanistic basis of the increased infectivity.

### Example 3: Validation of Vaccination Efficacy with Recombinant SARS-CoV-2 Spike Protein, Nucleic Acid, or Anti-Spike Antibody

To prove the applicability of a SARS-CoV-2 Spike protein-derived vaccine in vivo, an immunogenic antigen from a recombinant SARS-CoV-2 Spike protein, or fragment thereof; an antigen producing codon-optimized nucleic acid encoding a recombinant spike protein or fragment thereof; or an effector or neutralizing antibody that targets the spike protein is selected. A recombinant SARS-CoV-2 Spike protein isolated from engineered cells expressing a codon optimized SARS-CoV-2 Spike protein transcript is selected, along with an adjuvant, and is formulated into a vaccine containing pharmaceutical excipients to create a pharmaceutical composition. This composition is tested in mice for efficacy and safety before testing the composition in human subjects.

To ascertain whether the pharmaceutical vaccine compositions can elicit an effective response conferring immunity upon a subject against SARS-CoV-2 infection, a vaccine to is administered subjects via intramuscular injection. Multiple administrations are given to subjects in order to trigger a greater immune response. For mice, a mouse-infecting SARS-CoV-2 pseudovirus challenge is introduced a week after the last vaccine administration. Vaccine efficacy is monitored by anti-SARS-CoV-2 IgM and IgG antibody testing, PCR analysis for viral nucleic acids. To determine efficacy in human subjects, vaccinated individuals are compared to a controlled group of individuals who did not receive a vaccine living in similar environments and exhibiting similar behaviors exposing them to potential infections. Efficacy in mice challenged with SARS-CoV-2 pseudovirus is evaluated by infection comparison to mice that did not receive the vaccine but were similarly challenged with pseudovirus. Effective vaccine responses result in consistent negative PCR results for detecting viral nucleic acid, human symptom reports, and positive antibody response markers. Vaccine efficacy is further validated with serological assays that monitor the inhibition of SARS-CoV-2 viral entry in vitro in the presence of serum taken from vaccinated subjects.

### Example 4: Determining Toxicity of Recombinant SARS-CoV-2 Spike Peptides

To measure the dose-response toxicity of a SARS-CoV-2 spike peptide in vitro, a SARS-CoV-2 spike peptide is produced presenting viral particles from virus-like particles and incubated with healthy human cells. Dose-response determination of toxicity is rapidly achieved by plating lung organoids derived from patient iPS cells in multi-well plates amenable for both microscopy and fluorescent signal measurement. After the organoids have been incubated in the plates with growth media for a desired time to minimize extraneous environmental effects, known amounts of viral particles expressing the spike peptide are introduced in varying concentrations. After an effective incubation period to allow for infection, toxicity is determined.

Determinations of toxicity are made by extracting the lung organoid samples from the dish for further analysis and fixation to the dish for in-plate assays. An XTT solution (sodium 3′-[1-(phenylaminocarbonyl)-3,4-tetrazolium]-bis (4-methoxy6-nitro) benzene sulfonic acid hydrate) is added to the plates directly, causing low-viability cells of the organoid to catalyze the formation of a colorimetric dye indicative of oxidoreductase activity. The signal from the greater concentrations of such dye is read as proportional to the toxicity of the spike protein. Samples with varying concentrations of viral particles are analyzed together to generate a dose-response curve indicative of SARS-CoV-2 spike toxicity in lung organoids.

### Example 5: Identification of Therapeutic Models Against SARS-CoV-2 Via Viral Entry Assay

To screen for novel or repurposed chemical agents efficacious against SARS-CoV-2 infection, a SARS-CoV-2 spike peptide is produced in vitro presenting viral particles from virus-like particles incubated with healthy human cells to monitor viral entry into healthy human cells. These particles contain either fluorescent probes or protein tags that are detected and create a signal of viral infection in human cells or tissues. A baseline determination of viral entry and infection efficacy of such viral particles is achieved by plating lung organoids derived from patient iPS cells in multi-well plates amenable for both microscopy and fluorescent signal measurement. After the organoids have been incubated in the plates with growth media for a desired time to minimize extraneous environmental effects, known amounts of viral particles expressing the spike peptide are introduced in varying concentrations. After an effective incubation period to allow for infection, viral entry is monitored by detecting the presence of a fluorescent probe inside of the lung organoid.

To screen for agents that inhibit viral entry, varying concentrations of selected compounds are incubated or pre or post-viral addition with the lung organoids in any number of wells in order to determine efficacy and whether there is a dose-response curve. This determination is achieved by measuring the reduction in fluorescent signal in lung organoid cells as compared to organoids without compound incubation. These assays are performed in high throughput format allowing for the screening of millions of compounds with replication. From these results, new or repurposed chemical agents, including antibodies, are identified for further modification and study as potential therapeutics for SARS-CoV-2 infection.

### Example 6: Generation of a Transgenic Animal Model of SARS-CoV-2 Spike Protein Toxicity

To model the effects of SARS-CoV-2 spike protein toxicity in vivo, transgenic animal models are produced by introducing a codon-optimized SARS-CoV-2 spike transgene into the genome of a host. This is achieved by conventional animal model generation methods. Here, a zygote is isolated after HCG injection of a female mouse and subsequent breeding with a male mouse. The zygote is then injected with Cas nuclease enzymes, sgRNA's targeting a desired integration region, and gDNA donor plasmids containing a codon-optimized SARS-CoV-2 spike encoding transcript. After allowing for the resulting embryo to culture for 24-72 hours, the SARS-CoV-2 spike encoding transcript is integrated into the host genome and the embryo is implanted into a surrogate mother.

The SARS-CoV-2 spike encoding transcript is part of an expression cassette containing inducible promotors or operons that allow for controlled and tissue specific expression of the SARS-CoV-2 spike protein, specifically via a Tet-on system in lung epithelial tissue. Such a cassette allows one to achieve temporally controlled expression to monitor spike protein toxicity in the animal model by allowing the animal to develop without spike expression. Once the animal has reached adulthood, the promoter is induced and the animal's health may be monitored by behavioral studies, gene expression profiles taken from blood or tissue, and viability outcomes.

### Example 7: Gene Expression Profiling of Cells Expressing or Exposed to SARS-CoV-2 Spike Protein

To investigate genetic effects of SARS-CoV-2 Spike protein on human cells or tissues, spike protein expressing engineered cells, or spike protein exposed cells are analyzed for changes in gene expression relative to spike-naïve cells. Nucleic acids are extracted from two groups of cell lines, one having been incubated with SARS-CoV-2 spike protein, or SARS-CoV-2 spike protein containing viral particles, and the other group being naïve. Genetic sequences are analyzed according to conventional methods utilizing PCR, DNAseq, RNAseq, and DNA methylation sequencing.

The results of these studies are further analyzed using algorithms and high-order analysis, such as Monte Carlo methods, PCA, Hidden Markov models, or similar. Such analyses are used to identify useful patterns in determining possible therapeutic targets or biological effects important in cellular response to infection or SARS-CoV-2 Spike protein response.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

1.-175. (canceled)

176. A recombinant nucleic acid, wherein the recombinant nucleic acid encodes a SARS-CoV-2 spike protein or a fragment thereof comprising a nucleic acid sequence with at least 90% sequence identity to SEQ ID NO: 7 or a portion thereof, wherein the nucleic acid sequence comprises at least one silent mutation relative to SEQ ID NO: 7.

177. The recombinant nucleic acid of claim 176, wherein the nucleic acid sequence has at least 95% sequence identity to SEQ ID NO: 7.

178. The recombinant nucleic acid of claim 176, wherein the nucleic acid sequence comprising SEQ ID NO: 7 or a portion thereof comprises at least one mutation present in SEQ ID NO: 9.

179. The recombinant nucleic acid of claim 178, wherein the nucleic acid sequence comprising SEQ ID NO: 7 or a portion thereof comprises at least 3 mutations present in SEQ ID NO: 9.

180. The recombinant nucleic acid of claim 178, wherein the nucleic acid sequence comprising SEQ ID NO: 7 or a portion thereof comprises at least 5 mutations present in SEQ ID NO: 9.

181. The recombinant nucleic acid of claim 178, wherein the at least one silent mutation is a codon optimization for transcription in humans.

182. The recombinant nucleic acid of claim 178, wherein the at least one silent mutation reduces the frequency of homologous recombination between the recombinant nucleic acid and chromosomal DNA.

183. The recombinant nucleic acid of claim 178, wherein the at least one silent mutation reduces the frequency of repeat sequences in the recombinant nucleic acid.

184. The recombinant nucleic acid of claim 178, wherein the at least one silent mutation reduces the frequency of homologous recombination between the recombinant nucleic acid and viral DNA.

185. The recombinant nucleic acid of claim 178, wherein the at least one silent mutation reduces the frequency of homologous recombination between the recombinant nucleic acid and human DNA.

186. The recombinant nucleic acid of claim 178, wherein the at least one silent mutation reduces the frequency of homologous recombination between the recombinant nucleic acid and a second recombinant nucleic acid.

187. A recombinant nucleic acid encoding a SARS-CoV-2 spike protein or a fragment thereof, wherein the recombinant nucleic acid comprises a sequence with at least 90% sequence identity to SEQ ID NO: 9 or a portion thereof.

188. The recombinant nucleic acid of claim 187, wherein the recombinant nucleic acid comprises a sequence with at least 95% sequence identity to SEQ ID NO: 9 or a portion thereof.

189. The recombinant nucleic acid of claim 187, wherein the recombinant nucleic acid comprises the sequence set forth in SEQ ID NO: 9.

190. A pseudotyped lentiviral vector comprising (a) a pBOB-CAG vector, (b) a nucleic acid sequence encoding a SARS-CoV-2 spike protein or a fragment thereof comprising a nucleic acid sequence with at least 90% sequence identity to SEQ ID NO: 9, and (c) a protein tag.

191. The pseudotyped lentiviral vector of claim 190, wherein the pBOB-CAG vector comprises the nucleic acid sequence set forth in SEQ ID NO: 8.

192. The pseudotyped lentiviral vector of claim 190, wherein the protein tag is selected from the group consisting from ALFA-tag, AviTag, C-tag, Calmodulin-tag, polyglutamate tag, E-tag, FLAG-tag, His-tag, Myc-tag, NE-tag, Rho1D4-tag, S-tag, SBP-tag, Softag, HA-tag, Spot-tag, Strep-tag, T7-tag, TC tag, V5 tag, VSV-tag, Xpress tag, biotin carboxyl carrier protein, glutathione-S-transferase, GFP, HaloTag, SNAP-tag, CLIP-tag, HUH-tag, maltose binding protein, and thioredoxin.

193. The pseudotyped lentiviral vector of claim 190, wherein the protein tag comprises a sequence differing from SEQ ID NO: 5 by no more than three point mutations, deletions, or insertions.

194. The pseudotyped lentiviral vector of claim 190, wherein the nucleic acid further comprises at least one sequence selected from the group consisting of a promoter, an enhancer, a viral terminal repeat, a splice site, an origin of replication, a packaging signal, a recombination site, a sequence encoding an epitope recognizable by an antibody, a polyadenylation sequence, a sequence encoding at least one polypeptide, a sequence encoding at least one tRNA sequence, a sequence encoding at least one ribozyme, and a sequence encoding at least one RNA binding protein.

195. The pseudotyped lentiviral vector of claim 190, wherein the pseudotyped lentiviral vector comprises the nucleic acid sequence set forth in SEQ ID NO: 10.
